(19) United States
(12) Patent Application Publication
(10) Pub. No.: US 2025/0290843 A1
(43) Pub. Date: Sep. 18, 2025

(54) METHODS AND SYSTEMS FOR PREDICTING SINGLE CELL TRANSCRIPTOMIC INFORMATION FROM FLOW CYTOMETRY DATA

(71) Applicant: Melio Healthcare Limited, London (GB)

(72) Inventors: Adam Gregory LAING, London (GB); Thomas Stewart HAYDAY, London (GB); Duncan Robert MCKENZIE, London (GB); Jeremy Carter MASON, Cambridge (GB); Benjamin Peter THOMAS, London (GB); Eduardo DE PAIVA ALVES, London (GB); Hamed HASELI MASHHADI, Haverhill (GB); Hsiu Mien YANG, London (GB); Jack Andrew BIBBY, London (GB)

(73) Assignee: Melio Healthcare Limited, London (GB)

(21) Appl. No.: 19/080,024

(22) Filed: Mar. 14, 2025

Related U.S. Application Data
(60) Provisional application No. 63/565,485, filed on Mar. 14, 2024.

(51) Int. Cl. G01N 15/1429; G01N 15/10; G01N 15/14; G01N 21/64; G01N 33/569; G06N 20/00

(52) U.S. Cl. CPC G01N 15/1429; G01N 15/1459; G01N 21/6486; G01N 33/56972; G06N 20/00; G01N 2015/1006; G01N 2015/1402; G01N 2333/7051

(57) ABSTRACT

Method and systems for generating a single cell gene expression and/or clonality status profile for a subject from only flow cytometry data. Methods and systems for training a machine learning model to predict single cell gene expression and/or clonality status from flow cytometry and methods and systems to use the trained machine learning model to generate a single cell transcriptomic profile for a subject.

### CROSS-REFERENCE TO RELATED APPLICATIONS

This application claims the priority benefit of U.S. Provisional Patent Application No. 63/565,485, filed Mar. 14, 2024, the entire contents of which are incorporated herein by reference.

### FIELD

This disclosure relates to the prediction of single cell transcriptomic information from only flow cytometry data in order to generate a comprehensive single cell transcriptomic profile of a subject (e.g., human).

### BACKGROUND

Immune profiling, i.e., the analysis of a subject's immune health at the serological or cellular level at a given point in time, can aid in diagnosing immune-related diseases and disorders (e.g., allergies, overreactive immune responses (as in asthma and Crohn's disease (inflammatory bowel disease)), or autoimmune diseases (such as autoimmune polyglandular syndrome and some facets of diabetes; see, for example, “Diseases of the Immune System”, National Center for Biotechnology Information (US), in “Genes and Disease” [Internet], 1998-, Bethesda (MD)). Immune profiling can also be used to, e.g., identify an individual's specific response to infection diseases (e.g., viral, bacterial, fungal, or parasite infections), to monitor the response of patients, e.g., cancer patients, to treatment (see, e.g., Lyones, et al. (2017), “Immune Cell Profiling in Cancer: Molecular Approaches to Cell-Specific Identification,” Precision Oncology 1,26) and potentially, to predict healthcare outcomes.

Conventionally methods for generating an immune profile by immunophenotyping include, e.g., enzyme-linked immunosorbent assays (ELISAs), immunoblotting techniques, and flow cytometry-based techniques include the use of panels of fluorescently-labeled antibodies directed to a variety of cell surface receptors and manual gating of the flow cytometry data. These techniques are often laborious and time consuming and are not easily scalable to a level that enables the processing of hundreds or thousands of samples. Recently, high throughput manifestations of deep phenotyping methods such as full spectrum flow cytometry have been developed as cost-effective techniques for immune cell profiling. Both the conventional methods and the recent methods capture only a subset of the potentially useful information in patient samples.

Single cell transcriptomic profiling increases the resolution of immune profiles by providing exceptionally detailed information about the composition, state, and function of patient immune systems. The increased resolution is available because unlike immunophenotyping with flow cytometry that may profile a set of pre-selected surface proteins (on the order of 10-100), single cell transcriptomic profiles contain gene expression for all genes (approx. 20,000 genes). At such resolution, single cell transcriptomic immune profiling can improve our ability to understand the immune system in relation to health, disease, and vaccination.

Currently single cell transcriptomic profiling methods, such as single-cell RNA sequencing (scRNAseq), however, are prohibitively expensive, time consuming (sample processing taking multiple days), and are too low throughput to be used consistently on patient samples. Additionally, the data itself suffers from statistical limitations, as the relatively small number of individual cells that may be profiled in a sample limits power and introduces noise. Together these factors prevent scRNAseq being employed in diagnostics, patient monitoring, and population scale immune profiling despite the obvious advantage of its resolution.

### SUMMARY

Described herein are methods and systems to create, train and deploy machine learning (ML) models to predict single cell transcriptomics data from standardized flow cytometric immune profiling. Once trained on donor-matched flow cytometry and single cell RNA sequencing (scRNAseq) datasets, the methods described herein can be used to produce a detailed immune gene expression profile in the form of single cell transcriptomic data from standardized flow cytometry immune profiling data. The single cell transcriptomic data may comprise single cell expression quantifications, gene-set enrichment summaries and/or other data from immune receptor profiles such as clonal expansion statuses.

Disclosed herein are methods and systems for processing a sample, such as a blood sample, and generating a single cell transcriptomic immune profile for the subject. The disclosed methods and systems combine flow cytometry and a machine learning based approach to predict single cell transcriptomic data. The key advantages of the disclosed methods and systems are enabled by training and deploying ML models to generate single cell transcriptomic immune profiles in clinically relevant settings and timeframes.

In some embodiments, disclosed herein is a method for determining a single cell transcriptomic value from flow cytometry data, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cells from the sample; providing only at least a subset of the fluorescent intensity data, or data derived therefrom for the plurality of fluorescently-labeled cells as input to a machine learning model trained using single cell transcriptomics data for a first plurality of cells and pseudo-fluorescent data for the first plurality of cells; generating a single cell gene transcriptomic value for the fluorescently-labeled cells using the trained machine learning model.

In some embodiments, disclosed herein is a method for determining a single cell transcriptomic profile for a subject, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cell from the sample; providing only at least a subset of the fluorescent intensity data, or data derived therefrom for the plurality of fluorescently-labeled cells as input to a machine learning model trained using single cell transcriptomics data for a first plurality of cells and pseudo-fluorescent data for the first plurality of cells; generating a single cell transcriptomic value for the fluorescently-labeled cells using the trained machine learning model for a plurality of genes, thereby generating a single cell transcriptomic profile for the subject.

In some embodiments, the machine learning model is organized as a plurality of nodes, wherein each node comprises an individual machine learning model.

In some embodiments, the pseudo-fluorescent data for the first plurality of cells is generated by matching of at least a subset of protein marker data for each cell in the first plurality of cells to fluorescent intensity data for each cell in a second plurality of cells. In some embodiments, the matching at least a subset of protein markers comprises transforming the proteins markers data. In some embodiments, the transforming the protein marker data comprises a linear transformation. In some embodiments, the transforming the protein marker data comprises a non-linear transformation. In some embodiments, transforming the protein marker data comprises providing the protein marker data for at least a subset of the first plurality of cells and the fluorescent intensity data for at least a subset of the second plurality of cells to a machine learning model configured to transform the protein marker data for the subset of the first plurality of cells into pseudo-fluorescent marker data related to the fluorescent intensity data for the subset of the second plurality of cells and output the pseudo-fluorescent marker data for the subset of the cells in the first plurality of cells.

In some embodiments, the single cell transcriptomic data for the first plurality of cells is generated using a method for characterizing each cell in the first plurality of cells by simultaneous detection of a plurality of protein marker data and single cell transcriptomic values for a plurality of genes. In some embodiments, the transcriptomic data for the first plurality of cells is generated by immune receptor profiling.

In some embodiments, the protein marker data is generated by providing at least a subset of the single cell transcriptomic data for the first plurality of cells to a machine learning model configured to transform the at least a subset of single cell transcriptomic data for the first plurality of cells into protein marker data and output protein marker data for the first plurality of cells.

In some embodiments, the fluorescent intensity data for each cell in the second plurality of cells is generated using a flow cytometer to process fluorescently-labeled cells from the second plurality of cells. In some embodiments, the flow cytometer is configured for at least about 5, at least about 10, at least about 15, at least about 20, at least about 30, at least about 40, at least about 50, at least about 60, at least about 70, at least about 80, at least about 90, or at least about 100 fluorescence detection channels. In some embodiments, the flow cytometer is a full spectrum flow cytometer.

In some embodiments, the pseudo-fluorescent data for the first plurality of cells is pseudo-fluorescent cell classifications data for at least a subset of the first plurality of cells. In some embodiments, the pseudo-fluorescent data for the first plurality of cells further comprises pseudo-fluorescent cell classifications data for at least a subset of the first plurality of cells. In some embodiments, the pseudo-fluorescent cell classification data are generated by assigning a pseudo-fluorescent cell classification to each cell related to the protein marker data that corresponds to each cell in the first plurality of cells. In some embodiments, data derived from the fluorescent intensity data are flow cell classifications for each cell in the second plurality of cells.

In some embodiments, the machine model is trained to determine a single cell gene expression value.

In some embodiments, the single cell transcriptomic profile comprises a single cell transcriptomic value for at least about 100, about 1,000, about 5000, about 10000, or about 20,000 genes. In some embodiments, the single cell transcriptomic profile comprises emergent properties of single cell transcriptomic data relevant to groups of cells in the sample. In some embodiments, the emergent properties of single cell transcriptomic data relevant to groups of cells in the sample comprise gene signatures, cell trajectories and/or transcriptional patterns. In some embodiments, the single cell transcriptomic profile comprises emergent properties of single cell immune receptor profiling for groups of cells in the sample. In some embodiments, the emergent properties of single cell immune receptor profiling for groups of cells in the sample comprise immune receptor diversity and/or predictions of clonal expansions. In some embodiments, the single cell transcriptomic profile is used to diagnose an immune-related disease or disorder, monitor progression of an immune-related disease or disorder, or monitor a response to treatment of an immune-related disease or disorder in the subject.

In some embodiments, disclosed herein is a method for training a machine learning model to generate a predicted single cell transcriptomic value, comprising; collecting a population of cells comprising a first plurality of cells and a second plurality of cells; obtaining single cell transcriptomic data and protein marker data for each cell in the first plurality of cells, wherein the single cell transcriptomic data comprise a single cell transcriptomic value for a plurality of genes; obtaining fluorescent intensity data for each cell in the second plurality of cells generated using a flow cytometer to process fluorescently-labeled cells from the second plurality of cells; generating pseudo-fluorescent data by matching at least a subset of the protein marker data for each cell in the first plurality of cells to the fluorescent intensity data for each cell in the second plurality of cells; and training a machine learning model to generate a predicted single cell transcriptomic value, wherein the training is based on the transcriptomic data for at least a subset of the first plurality of cells and the pseudo-fluorescent data for the at least a subset of the first plurality of cells.

In some embodiments, the single cell transcriptomic data and protein marker data for each cell in the first plurality of cells are generated using a method for characterizing a cell by simultaneous detection of a plurality of protein markers and single cell transcriptomic values for a plurality of genes. In some embodiments, the single cell transcriptomic data for the first plurality of cells is generated by immune receptor profiling.

In some embodiments, the protein marker data is generated by providing at least a subset of the single cell transcriptomic data for the first plurality of cells to a machine learning model configured to transform the at least a subset of single cell transcriptomic data for the first plurality of cells into protein marker data and output protein marker data for the first plurality of cells.

In some embodiments, disclosed herein is a method for training a machine learning model to generate a predicted single cell transcriptomic value, comprising; collecting a population of cells, comprising a first plurality of cells and a second plurality of cells; obtaining single cell transcriptomic data and protein marker data for each cell in the first plurality of cells, wherein the single cell transcriptomic data comprise a transcriptomic value for a first plurality of genes, wherein the protein marker data comprise a marker value for a plurality of surface proteins, assigning a marker cell classification for each cell related to the protein marker data that corresponds to each cell in the first plurality of cells; determining a flow cell classification for each cell in the second plurality of cells by processing data generated using a flow cytometer to process fluorescently-labeled cells from the second plurality of cells generate pseudo-fluorescent cell classification data by matching the marker cell classifications for each cell in the first plurality of cells to the flow cell classifications for each cell in the second plurality of cells; and training a machine learning model to generate a predicted single cell transcriptomic value, wherein the training is based on the transcriptomic data for at least a subset of the first plurality of cells and the pseudo-fluorescent cell classification data for the at least a subset of the first plurality of cells.

In some embodiments, the single cell transcriptomic data and protein marker data for each cell in the first plurality of cells are generated using a method for characterizing a cell by simultaneous detection of a plurality of protein markers and transcriptomic values for a plurality of genes.

In some embodiments, the protein marker data comprise an antibody-derived tag (ADT) marker value for a plurality of surface proteins. In some embodiments, the single cell transcriptomic data for the first plurality of cells is generated by immune receptor profiling.

In some embodiments, the protein marker data is generated by providing at least a subset of the single cell transcriptomic data for the first plurality of cells to a machine learning model configured to transform the at least a subset of single cell transcriptomic data for the first plurality of cells into protein marker data and output protein marker data for the first plurality of cells.

In some embodiments, disclosed herein is a method for training a machine learning model to generate a predicted single cell transcriptomic value for a plurality of genes, comprising; collecting a population of cells comprising a first plurality of cells and a second plurality of cells; obtaining single cell transcriptomic data and protein marker data for each cell in the first plurality of cells, wherein the single cell transcriptomic data comprise a transcriptomic value for a plurality of genes, wherein the protein marker data comprise a value for a plurality of surface marker proteins; obtaining fluorescent intensity data for each cell in the second plurality of cells using a flow cytometer to process fluorescently-labeled cells from the second plurality of cells, wherein the fluorescent intensity data comprise fluorescence values for the plurality of surface marker proteins; generating pseudo-fluorescent markers for the first plurality of cells by transforming the at least a subset of the protein marker data for the first plurality of cells into pseudo-fluorescent markers data related to the fluorescent intensity data for the subset of the second plurality of cells; and training a machine learning model to generate a predicted single cell transcriptomic value, wherein the training is based on the single cell transcriptomic data for at least a subset of the first plurality of cells and the pseudo-fluorescent cell markers data for the at least a subset of the first plurality of cells.

In some embodiments, the single cell transcriptomic data and protein marker data for each cell in the first plurality of cells are generated using a method for characterizing a cell by simultaneous detection of a plurality of protein markers and transcriptomic values for a plurality of genes.

In some embodiments, the protein marker data comprise an ADT marker value for a plurality of surface proteins. In some embodiments, the single cell transcriptomic data for the first plurality of cells is generated by immune receptor profiling.

In some embodiments, the protein marker data is generated by providing at least a subset of the single cell transcriptomic data for the first plurality of cells to a machine learning model configured to transform the at least a subset of single cell transcriptomic data for the first plurality of cells into protein marker data and output protein marker data for the first plurality of cells. In some embodiments, the transforming the protein marker data comprises a linear transformation. In some embodiments, the transforming the protein marker data comprises a non-linear transformation. In some embodiments, the transforming the protein marker data comprises providing the protein marker data for at least a subset of the first plurality of cells and the fluorescent intensity data for at least a subset of the second plurality of cells to a machine learning model configured to transform the protein marker data for the subset of the first plurality of cells into pseudo-fluorescent marker data related to the fluorescent intensity data for the subset of the second plurality of cells and output the pseudo-fluorescent marker data for the subset of the cells in the first plurality of cells.

In some embodiments, disclosed herein is a method for training a machine learning model to generate a predicted single cell transcriptomic value for a plurality of genes, comprising; collecting a population of cells comprising a first plurality of cells and a second plurality of cells; obtaining single cell transcriptomic data and protein marker data for each cell in the first plurality of cells, wherein the single cell transcriptomic data comprise a transcriptomic value for a plurality of genes, wherein the protein marker data comprise a marker value for a plurality of surface marker proteins; assigning a marker cell classification for each cell related to the protein marker data that corresponds to each cell in the first plurality of cells; obtaining fluorescent intensity data for each cell in the second plurality of cells using a flow cytometer to process fluorescently-labeled cells from the second plurality of cells wherein the fluorescent intensity data a fluorescent intensity value for the plurality of surface marker proteins; determining a flow cell classification for each cell in the second plurality of cells by processing the flow intensity data; generating pseudo-fluorescent cell data by matching the marker cell classifications for each cell in the first plurality of cells to the flow cell classifications for each cell in the second plurality of cells; generating pseudo-fluorescent markers for the first plurality of cells by transforming at least a subset of the protein marker data for the first plurality of cells into pseudo-fluorescent markers data related to the fluorescent intensity data for the subset of the second plurality of cells; and training a machine learning model to generate a predicted single cell gene expression value, wherein the training is based on the single cell transcriptomic data for at least a subset of the first plurality of cells, the pseudo-fluorescent cell markers data for the at least a subset of the first plurality of cells, and the pseudo-fluorescent cell classification data for the at least a subset of the first plurality of cells.

In some embodiments, the single cell transcriptomic data and protein marker data for each cell in the first plurality of cells are generated using a method for characterizing a cell by simultaneous detection of a plurality of protein markers and transcriptomic values for a plurality of genes. In some embodiments, the protein marker data comprise an ADT marker value for a plurality of surface proteins. In some embodiments, the single cell transcriptomic data for the first plurality of cells is generated by immune receptor profiling.

In some embodiments, the protein marker data is generated by providing at least a subset of the single cell transcriptomic data for the first plurality of cells to a machine learning model configured to transform the at least a subset of single cell transcriptomic data for the first plurality of cells into protein marker data and output protein marker data for the first plurality of cells. In some embodiments, the transforming the protein marker data comprises a linear transformation.

In some embodiments, the transforming the protein marker data comprises a non-linear transformation. In some embodiments, the transforming the protein marker data comprises providing the protein marker data for at least a subset of the first plurality of cells and the fluorescent intensity data for at least a subset of the second plurality of cells to a machine learning model configured to transform the protein marker data for the subset of the first plurality of cells into pseudo-fluorescent markers data related to the fluorescent intensity data for the subset of the second plurality of cells and output the pseudo-fluorescent markers data for the subset of the cells in the first plurality of cells.

In some embodiments, the machine learning model is organized in a cascading hierarchical tree structure comprising a plurality of nodes, and wherein each node comprises an individual machine learning model. In some embodiments, each individual machine learning model comprises a neural network model. In some embodiments, each individual machine learning model comprises a gradient boosting tree model. In some embodiments, the plurality of nodes comprises at least 1000, 1200, 1400, 1600, 1800, 2000, 2200, or 2400 nodes.

In some embodiments, the flow cytometer is configured for at least about 5, at least about 10, at least about 15, at least about 20, at least about 30, at least about 40, at least 50, at least about 60, at least about 70, at least about 80, at least about 90, or at least about 100 fluorescence detection channels. In some embodiments, the flow cytometer is a full spectrum flow cytometer.

In some embodiments, the single cell transcriptomic value comprises a transcriptional profile for the single cell comprising emergent properties of single cells transcriptomic data. In some embodiments, the emergent properties of single transcriptomic data comprise cell trajectories and/or transcriptional patterns. In some embodiments, the single cell transcriptomic value comprises emergent properties of a single cell immune cell receptor profile. In some embodiments, wherein the emergent properties comprise immune receptor diversity and/or prediction of clonal expansions.

In some embodiments, disclosed herein is a method for generating a single cell transcriptomic profile for a subject, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cells from the sample; providing only at least a subset of the fluorescent intensity data, or data derived therefrom for the plurality of fluorescently-labeled cells as input to a machine learning model trained using single cell transcriptomics data for a first plurality of cells and pseudo-fluorescent data for the first plurality of cells; generating a predicted single cell transcriptomic value for the fluorescently-labeled cells using the trained machine learning model, thereby generating a single cell transcriptomic profile for the subject.

In some embodiments, the pseudo-fluorescent data for the first plurality of cells is generated by matching at least a subset of protein marker data for each cell in the first plurality of cells to fluorescent intensity data for each cell in a second plurality of cells.

In some embodiments, the pseudo-fluorescent data comprises pseudo-fluorescent marker data for the first plurality of cells and/or pseudo-fluorescent cell classifications for the first plurality of cells.

In some embodiments, the matching at least a subset of protein markers comprises transforming the protein marker data into pseudo-fluorescent marker data. In some embodiments, the transforming the protein marker data comprises a non-linear transformation. In some embodiments, the transforming the protein marker data comprises providing the protein marker data for at least a subset of the first plurality of cells and the fluorescent intensity data for at least a subset of the second plurality of cells to a machine learning model configured to transform the protein marker data for the subset of the first plurality of cells into pseudo-fluorescent markers data related to the fluorescent intensity data for the subset of the second plurality of cells and output the pseudo-fluorescent markers data for the subset of the cells in the first plurality of cells.

In some embodiments, the pseudo-fluorescent cell classification data are generated by assigning a pseudo-fluorescent cell classification to each cell related to the protein marker data that corresponds to each cell in the first plurality of cells.

In some embodiments, the single cell transcriptomics data for the first plurality of cells is generated using a method for characterizing each cell in the first plurality of cells by simultaneous detection of a plurality of protein marker data and single cell transcriptomic values. In some embodiments, the protein marker data comprise an ADT marker value for a plurality of surface proteins. In some embodiments, the single cell transcriptomic data for the first plurality of cells is generated by immune receptor profiling. In some embodiments, the immune receptor profiling comprises TCR sequencing. In some embodiments, the immune receptor profiling comprises BCR sequencing. In some embodiments, the method for characterizing each cell is CITE-seq.

In some embodiments, the single cell transcriptomics data comprises a single cell transcriptomic quantification and/or a clonal expansion status. In some embodiments, the clonal expansion status for the first plurality of cells are generated from immune receptor profiling data. In some embodiments, the immune receptor profiling data comprises TCR and/or BCR sequence data.

In some embodiments, the single cell transcriptomic profile comprises single cell transcriptomic quantifications for a plurality of genes and/or clonal expansion statuses for a plurality of the fluorescently labeled cells. In some embodiments, the flow cytometer is configured for at least about 5, at least about 10, at least about 15, at least about 20, at least about 30, at least about 40, at least 50, at least about 60, at least about 70, at least about 80, at least about 90, or at least about 100 fluorescence detection channels. In some embodiments, the flow cytometer is a full spectrum flow cytometer.

In some embodiments, the trained machine learning model comprises a neural network, a hybrid classifier regression multilayer neural network, a regression multilayer neural network, a tweedie regression, a hybrid mean standard error (MSE)/Tweedie neural network, a gradient descent model, or an XGboost model.

In some embodiments, disclosed herein is a method for generating a single cell transcriptomic profile for a subject, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cells from the sample; providing only at least a subset of the fluorescent intensity data, or data derived therefrom for the plurality of fluorescently-labeled cells as input to a machine learning model trained using single cell transcriptomics data for a first plurality of cells and pseudo-fluorescent marker data for the first plurality of cells; generating a predicted single cell transcriptomic quantification for the fluorescently-labeled cells using the trained machine learning model, thereby generating a single cell transcriptomic profile for the subject. In some embodiments, the single cell transcriptomic data comprises single cell transcriptomic quantifications for a plurality of genes.

In some embodiments, disclosed herein is a method for generating a single cell transcriptomic profile for a subject, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cells from the sample; providing only at least a subset of the fluorescent intensity data, or data derived therefrom for the plurality of fluorescently-labeled cells as input to a machine learning model trained using single cell transcriptomics data for a first plurality of cells and pseudo-fluorescent cell-classifications for the first plurality of cells; generating a predicted single cell transcriptomic quantification for the fluorescently-labeled cells using the trained machine learning model, thereby generating a single cell transcriptomic profile for the subject. In some embodiments, the single cell transcriptomic data comprises single cell transcriptomic quantifications for a plurality of genes.

In some embodiments, disclosed herein is a method for generating a single cell transcriptomic profile for a subject, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cells from the sample; providing only at least a subset of the fluorescent intensity data, or data derived therefrom for the plurality of fluorescently-labeled cells as input to a machine learning model trained using single cell transcriptomics data for a first plurality of cells and pseudo-fluorescent marker data and pseudo-fluorescent cell-classifications for the first plurality of cells; generating a predicted single cell transcriptomic quantification for the fluorescently-labeled cells using the trained machine learning model, thereby generating a single cell transcriptomic profile for the subject. In some embodiments, the single cell transcriptomic data comprises single cell transcriptomic quantifications for a plurality of genes.

In some embodiments, disclosed herein is a method for generating a single cell transcriptomic profile for a subject, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cells from the sample; providing only at least a subset of the fluorescent intensity data, or data derived therefrom for the plurality of fluorescently-labeled cells as input to a machine learning model trained using single cell transcriptomics data, comprising clonal expansion statutes, for a first plurality of cells and pseudo-fluorescent marker data for the first plurality of cells; generating a predicted clonal expansion status for the fluorescently-labeled cells using the trained machine learning model, thereby generating a single cell transcriptomic profile for the subject.

In some embodiments, disclosed herein is a method for generating a single cell transcriptomic profile for a subject, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cells from the sample; providing only at least a subset of the fluorescent intensity data, or data derived therefrom for the plurality of fluorescently-labeled cells as input to a machine learning model trained using single cell transcriptomics data, comprising clonal expansion statutes, for a first plurality of cells and pseudo-fluorescent cell-classifications for the first plurality of cells; generating a predicted a predicted clonal expansion status for the fluorescently-labeled cells using the trained machine learning model, thereby generating a single cell transcriptomic profile for the subject.

In some embodiments, disclosed herein is a method for generating a single cell transcriptomic profile for a subject, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cells from the sample; providing only at least a subset of the fluorescent intensity data, or data derived therefrom for the plurality of fluorescently-labeled cells as input to a machine learning model trained using single cell transcriptomics data, comprising clonal expansion statutes, for a first plurality of cells and pseudo-fluorescent marker data and pseudo-fluorescent cell-classifications for the first plurality of cells; generating a predicted clonal expansion status for the fluorescently-labeled cells using the trained machine learning model, thereby generating a single cell transcriptomic profile for the subject.

In some embodiments, provided herein is a method for generating a machine learning model to generate a predicted single cell transcriptomic value, comprising; collecting a population of cells comprising a first plurality of cells and a second plurality of cells; obtaining single cell transcriptomic data and protein marker data for each cell in the first plurality of cells, wherein the single cell transcriptomic data comprise a single cell transcriptomic value for a plurality of genes; obtaining fluorescent intensity data for each cell in the second plurality of cells generated using a flow cytometer to process fluorescently-labeled cells from the second plurality of cells; generating pseudo-fluorescent data by matching at least a subset of the protein marker data for each cell in the first plurality of cells to the fluorescent intensity data for each cell in the second plurality of cells; and training a machine learning model to generate a predicted single cell transcriptomic value, wherein the training is based on the transcriptomic data for at least a subset of the first plurality of cells and the pseudo-fluorescent data for the at least a subset of the first plurality of cells.

In some embodiments, obtaining single cell transcriptomic data and protein marker data for each cell in the first plurality of cells comprises simultaneous detection of a plurality of protein marker data and single cell transcriptomic values.

In some embodiments, the pseudo-fluorescent data comprises pseudo-fluorescent marker data for the first plurality of cells and/or pseudo-fluorescent cell classifications for the first plurality of cells. In some embodiments, the matching at least a subset of protein markers comprises transforming the protein marker data into pseudo-fluorescent marker data. In some embodiments, the transforming the protein marker data comprises a non-linear transformation. In some embodiments, the transforming the protein marker data comprises providing the protein marker data for at least a subset of the first plurality of cells and the fluorescent intensity data for at least a subset of the second plurality of cells to a machine learning model configured to transform the protein marker data for the subset of the first plurality of cells into pseudo-fluorescent markers data related to the fluorescent intensity data for the subset of the second plurality of cells and output the pseudo-fluorescent markers data for the subset of the cells in the first plurality of cells.

In some embodiments, the pseudo-fluorescent cell classification data are generated by assigning a pseudo-fluorescent cell classification to each cell related to the protein marker data that corresponds to each cell in the first plurality of cells.

In some embodiments, obtaining single cell transcriptomic data and protein marker data for each cell in the first plurality of cells, comprises use of a method for characterizing each cell in the first plurality of cells by simultaneous detection of a plurality of protein marker data and single cell transcriptomic values. In some embodiments, the protein marker data comprise an ADT marker value for a plurality of surface proteins. In some embodiments, the single cell transcriptomic data for the first plurality of cells is generated by immune receptor profiling. In some embodiments, the immune receptor profiling comprises TCR sequencing. In some embodiments, the immune receptor profiling comprises BCR sequencing. In some embodiments, the method for characterizing each cell is CITE-seq. In some embodiments, the single cell transcriptomic data further comprises clonal expansion statuses for the first plurality of cells.

In some embodiments, the machine learning model has a flexible architecture.

In some embodiments, the single cell transcriptomic values comprise single cell transcriptomic quantifications for the plurality of genes. In some embodiments, the method comprises selecting a machine learning model architecture from a group of machine learning architectures based on a distribution of the single cell transcriptomic quantifications for the plurality of genes. In some embodiments, the group of machine learning architecture as comprises a hybrid classifier regression multilayer neural network, a regression multilayer neural network, a tweedie regression, a hybrid mean standard error (MSE)/Tweedie neural network and a gradient descent model. In some embodiments, the machine learning model is trained to predict a single cell transcriptomic quantification from fluorescent intensity data. In some embodiments, training the machine learning model comprises, minimizing one or more loss functions based on the machine learning model architecture. In some embodiments, the one or more loss functions are selected from a group consisting of a log likelihood loss based on a tweedie distribution, negative log likelihood loss, mean squared error loss, mean absolute error (MAE), and cross entropy loss.

In some embodiments, the distribution of the single cell transcriptomic quantifications for the plurality of genes comprises a normal distribution and a binary distribution. In some embodiments, the method comprises selecting a hybrid classifier regression multilayer neural network based on a distribution of the single cell transcriptomic quantifications for the plurality of genes. In some embodiments, training the machine learning model comprises, minimizing a mean squared error loss function and a cross entropy loss function. In some embodiments, the plurality of genes are highly expressed genes.

In some embodiments, the distribution of the single cell transcriptomic quantifications for the plurality of genes comprises a tweedie distribution and a binary distribution. In some embodiments, the method comprises selecting a regression multilayer neural network based on a distribution of the single cell transcriptomic quantifications for the plurality of genes. In some embodiments, training the machine learning model comprises minimizing a negative log-likelihood loss based on the tweedie distribution and a cross entropy loss for the binary distribution. In some embodiments, the plurality of genes are highly variable genes.

In some embodiments, the distribution of the single cell transcriptomic quantifications for the plurality of genes comprises a tweedie distribution and a binary distribution. In some embodiments, the method comprises selecting a hybrid neural network based on a distribution of the single cell transcriptomic quantifications for the plurality of genes. In some embodiments, the method comprises selecting a tweedie regression based on a distribution of the single cell transcriptomic quantifications for the plurality of genes. In some embodiments, the method comprises selecting a hybrid neural network and a tweedie regression based on a distribution of the single cell transcriptomic quantifications for the plurality of genes. In some embodiments, the plurality of genes are highly variable genes.

In some embodiments, the plurality of genes comprises multiple subsets of genes with different distributions of single cell transcriptomic quantifications. In some embodiments, the method comprises selecting a hybrid mean standard error (MSE)/tweedie neural network based on a distributions of the single cell transcriptomic quantifications for the plurality of genes. In some embodiments, training the machine learning model comprises minimizing a mean squared error loss for a subset of highly expressed genes, negative log-likelihood loss for a subset of low expressed genes, and two cross entropy losses. In some embodiments, the plurality of genes are non-negligible genes.

In some embodiments, the single cell transcriptomic values comprise clonal expansion statuses. In some embodiments, the machine learning model is a classifier model trained to predict clonal expansion statuses from fluorescence intensity data. In some embodiments, the classifier model is an XGboost classifier. In some embodiments, the predicted single cell transcriptomic value comprises predicted clonal expansion statuses for the second plurality of cells.

In some embodiments, the flow cytometer is configured for at least about 5, at least about 10, at least about 15, at least about 20, at least about 30, at least about 40, at least 50, at least about 60, at least about 70, at least about 80, at least about 90, or at least about 100 fluorescence detection channels. In some embodiments, the flow cytometer is a full spectrum flow cytometer.

In some embodiments, provided herein is a method for training a machine learning model to generate a predicted single cell transcriptomic quantification, comprising; collecting a population of cells comprising a first plurality of cells and a second plurality of cells; obtaining single cell transcriptomic data and protein marker data for each cell in the first plurality of cells, wherein the single cell transcriptomic data comprise a single cell transcriptomic quantification for a plurality of genes; obtaining fluorescent intensity data for each cell in the second plurality of cells generated using a flow cytometer to process fluorescently-labeled cells from the second plurality of cells; generating pseudo-fluorescent marker data by matching at least a subset of the protein marker data for each cell in the first plurality of cells to the fluorescent intensity data for each cell in the second plurality of cells; and training a machine learning model to generate a predicted single cell transcriptomic quantification, wherein the training is based on the transcriptomic data for at least a subset of the first plurality of cells and the pseudo-fluorescent marker data for the at least a subset of the first plurality of cells. In some embodiments, the machine learning model has a flexible architecture. In some embodiments, the methods comprise selecting a machine learning model architecture from a group of machine learning architectures based on a distribution of the single cell transcriptomic quantifications for the plurality of genes.

In some embodiments, provided herein is a method for training a machine learning model to generate a predicted single cell transcriptomic quantification, comprising; collecting a population of cells comprising a first plurality of cells and a second plurality of cells; obtaining single cell transcriptomic data and protein marker data for each cell in the first plurality of cells, wherein the single cell transcriptomic data comprise a single cell transcriptomic quantification for a plurality of genes; obtaining fluorescent intensity data for each cell in the second plurality of cells generated using a flow cytometer to process fluorescently-labeled cells from the second plurality of cells; generating pseudo-fluorescent cell classifications by matching at least a subset of the protein marker data for each cell in the first plurality of cells to the fluorescent intensity data for each cell in the second plurality of cells; and training a machine learning model to generate a predicted single cell transcriptomic quantification, wherein the training is based on the transcriptomic data for at least a subset of the first plurality of cells and the pseudo-fluorescent cell classifications for the at least a subset of the first plurality of cells. In some embodiments, the machine learning model has a flexible architecture. In some embodiments, the methods comprise selecting a machine learning model architecture from a group of machine learning architectures based on a distribution of the single cell transcriptomic quantifications for the plurality of genes.

In some embodiments, provided herein is a method for training a machine learning model to generate a predicted single cell transcriptomic quantification, comprising; collecting a population of cells comprising a first plurality of cells and a second plurality of cells; obtaining single cell transcriptomic data and protein marker data for each cell in the first plurality of cells, wherein the single cell transcriptomic data comprise a single cell transcriptomic quantification for a plurality of genes; obtaining fluorescent intensity data for each cell in the second plurality of cells generated using a flow cytometer to process fluorescently-labeled cells from the second plurality of cells; generating pseudo-fluorescent marker data and pseudo-fluorescent cell classifications by matching at least a subset of the protein marker data for each cell in the first plurality of cells to the fluorescent intensity data for each cell in the second plurality of cells; and training a machine learning model to generate a predicted single cell transcriptomic quantification, wherein the training is based on the transcriptomic data for at least a subset of the first plurality of cells and the pseudo-fluorescent marker data and pseudo-fluorescent cell classifications for the at least a subset of the first plurality of cells. In some embodiments, the machine learning model has a flexible architecture. In some embodiments, the methods comprise selecting a machine learning model architecture from a group of machine learning architectures based on a distribution of the single cell transcriptomic quantifications for the plurality of genes.

In some embodiments, provided herein is a method for training a machine learning model to generate predicted clonal expansion statuses, comprising; collecting a population of cells comprising a first plurality of cells and a second plurality of cells; obtaining single cell transcriptomic data and protein marker data for each cell in the first plurality of cells, wherein the single cell transcriptomic data comprises single cell clonal expansion statuses for the first plurality of cells; obtaining fluorescent intensity data for each cell in the second plurality of cells generated using a flow cytometer to process fluorescently-labeled cells from the second plurality of cells; generating pseudo-fluorescent marker data by matching at least a subset of the protein marker data for each cell in the first plurality of cells to the fluorescent intensity data for each cell in the second plurality of cells; and training a machine learning model to generate predicted clonal expansion statuses, wherein the training is based on the transcriptomic data for at least a subset of the first plurality of cells and the pseudo-fluorescent marker data for the at least a subset of the first plurality of cells. In some embodiments, the machine learning model is a classifier model trained to predict clonal expansion statuses from fluorescence intensity data.

In some embodiments, provided herein is a method for training a machine learning model to generate a predicted clonal expansion statuses, comprising; collecting a population of cells comprising a first plurality of cells and a second plurality of cells; obtaining single cell transcriptomic data and protein marker data for each cell in the first plurality of cells, wherein the single cell transcriptomic data comprises single cell clonal expansion statuses for the first plurality of cells; obtaining fluorescent intensity data for each cell in the second plurality of cells generated using a flow cytometer to process fluorescently-labeled cells from the second plurality of cells; generating pseudo-fluorescent cell classifications by matching at least a subset of the protein marker data for each cell in the first plurality of cells to the fluorescent intensity data for each cell in the second plurality of cells; and training a machine learning model to generate predicted clonal expansion statuses, wherein the training is based on the transcriptomic data for at least a subset of the first plurality of cells and the pseudo-fluorescent cell classifications for the at least a subset of the first plurality of cells. In some embodiments, the machine learning model is a classifier model trained to predict clonal expansion statuses from fluorescence intensity data.

In some embodiments, provided herein is a method for training a machine learning model to generate a predicted clonal expansion statuses, comprising; collecting a population of cells comprising a first plurality of cells and a second plurality of cells; obtaining single cell transcriptomic data and protein marker data for each cell in the first plurality of cells, wherein the single cell transcriptomic data comprises single cell clonal expansion statuses for the first plurality of cells; obtaining fluorescent intensity data for each cell in the second plurality of cells generated using a flow cytometer to process fluorescently-labeled cells from the second plurality of cells; generating pseudo-fluorescent marker data and pseudo-fluorescent cell classifications by matching at least a subset of the protein marker data for each cell in the first plurality of cells to the fluorescent intensity data for each cell in the second plurality of cells; and training a machine learning model to generate clonal expansion statuses, wherein the training is based on the transcriptomic data for at least a subset of the first plurality of cells and the pseudo-fluorescent marker data and pseudo-fluorescent cell classifications for the at least a subset of the first plurality of cells. In some embodiments, the machine learning model is a classifier model trained to predict clonal expansion statuses from fluorescence intensity data.

### BRIEF DESCRIPTION OF THE FIGURES

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawing of which:

FIG. 1 shows a non-limiting simplified schematic for predicting a transcriptomic value from sample cells.

FIG. 2 shows a non-limiting simplified schematic for training machine learning model with single cell transcriptomic data and protein marker data matched to fluorescent data.

FIG. 3 shows a non-limiting simplified schematic for training machine learning model with single cell transcriptomic data marker cells classifications that have been matched to flow cell classification to generate pseudo-fluorescent classification.

FIG. 4 shows a non-limiting simplified schematic for training machine learning model with single cell transcriptomic data and protein marker data transformed into pseudo-fluorescent marker data.

FIG. 5 shows a non-limiting example of a process diagram for training machine learning model with single cell transcriptomic data, marker cell classifications that have been matched to flow cell classifications, and protein marker data transformed into pseudo-fluorescent marker data.

FIG. 6 shows a non-limiting schematic for training a machine learning model with single cell transcriptomic data and protein marker data matched to fluorescent data.

FIG. 7A-7B shows a non-limiting schematic of a method according to some of the methods and systems described herein. FIG. 7A shows exemplary training data collection and training data processing modules. FIG. 7B shows exemplary model training model evaluation and model deployment modules.

FIG. 8 illustrates an exemplary computing system, in accordance with some of the embodiments and systems described herein.

FIG. 9A and FIG. 9B show flow cytometry classifications from fluorescent markers (FIG. 9A) and pseudo-fluorescent classification from ADT markers (FIG. 9B).

FIG. 10 shows alignment of cell classification frequencies derived from flow cytometry and from ADT markers.

FIG. 11 shows correlation between predicted and actual transcriptomic values for 1000 genes. Prediction done with ADT marker values.

FIG. 12 shows correlation between predicted and actual transcriptomic values for 1000 genes. Prediction done with ADT cell classifications.

FIG. 13A and FIG. 13B show neural network architecture. FIG. 13A shows a neural network architecture with one latent layer. FIG. 13B shows a neural network architecture with one latent layer flowing into another neural network with one hidden layer.

FIG. 14 shows an example boosted tree model architecture with 3 trees.

FIG. 15A and FIG. 15B show ADT marker data and FSFC data in UMAP space before harmonization (FIG. 15A) and after harmonization (FIG. 15B).

FIG. 16A and FIG. 16B show ADT marker data before and after correction compared to flow data. FIG. 16A shows this distribution of CD28. FIG. 16B shows this distribution of CCR7.

FIG. 17A and FIG. 17B show the correlation between pseudo-fluorescent cell classifications (ADT ratios) and flow cell classifications (flow ratios) before filtering (FIG. 17A) and after filtering (FIG. 17B).

FIG. 18A and FIG. 18B show the correlation between pseudo-fluorescent marker data (ADT MFIs) and flow marker data (Flow MFIs) before filtering (FIG. 18A) and after filtering (FIG. 18B).

FIG. 19 shows the mean absolute error in the prediction from the neural network model trained with pseudo-fluorescent marker data for all genes.

FIG. 20A and FIG. 20B shows the actual (measured) single cell gene expression and the predicted gene expression in UMAP space overlayed (FIG. 20A) and side by side (FIG. 20B).

FIG. 21A, FIG. 21B, FIG. 21C, FIG. 21D, FIG. 21E, FIG. 21F, FIG. 21G, FIG. 21H, FIG. 21I, FIG. 21J, and FIG. 21k, show the correlation between true single cell gene expression and single cell gene expression predicted from flow intensities from the T panel markers. Each panel of the figure is a sample and MSE annotations represent mean squared error evaluation of each gene mean per broad cell lineage.

FIG. 22A, FIG. 22B, FIG. 22C, FIG. 22D, FIG. 22E, FIG. 22F, FIG. 22G, and FIG. 22H show the correlation between true single cell gene expression and single cell gene expression predicted from flow intensities from the T panel markers filtered for cells classifications with MAE <0.1. Each panel of the figure is a sample and MSE annotations represent mean squared error evaluation of each gene mean per cell type (from all matched pseudo-fluorescent classifications).

FIG. 23A and FIG. 23B shows the actual (measured) single cell gene expression and the predicted gene expression in UMAP space side by side (FIG. 23A) and overlayed (FIG. 23B).

FIG. 24A and FIG. 24B show predicted single cell gene expression in UMAP space, colored by cell cluster (FIG. 24A) and by expression of expected marker gene (FIG. 24B).

FIG. 25 shows clustering of T cell genes and their locations on a cell correlation heatmap.

FIG. 26A and FIG. 26B shows pathway analysis results for the predicted data (FIG. 26A) and validation data (FIG. 26B).

FIG. 27A and FIG. 27B show predicted single cell gene expression data in UMAP space. FIG. 27A shows membership to different cell classifications for 5 samples. FIG. 27B shows membership according to different marker genes.

FIG. 28A-28D show clustering of single cells based on predicted single cell gene expression. The predictions are based on Based on predicted single cell gene expression according to one of four model architectures, hybrid neural network (FIG. 28A), hybrid neural network and tweedie regression (FIG. 28B), Tweedie regression (FIG. 28C), tweedie regression trained with variable genes and highly expressed genes (FIG. 28D).

FIG. 29A, FIG. 29B, FIG. 29C, FIG. 29D, FIG. 29E, FIG. 29F, FIG. 29G, FIG. 29H, FIG. 29I, FIG. 29J show a correlation between true single cell gene expression and predicted single cell gene expression from flow intensities from the A panel. Each panel of the figure is a sample and MSE annotations represent mean squared error evaluation of each gene mean per cell type.

FIG. 30A, FIG. 30B, FIG. 30C, FIG. 30D, FIG. 30E, FIG. 30F, FIG. 30G, FIG. 30H, FIG. 30I and FIG. 30J show a correlation between true single cell gene expression and predicted single cell gene expression from flow intensities from the T panel. Each panel of the figure is a sample and MSE annotations represent mean squared error evaluation of each gene mean per cell type.

FIG. 31 shows predicted gene expression in UMAP space, highlighting according to known population marker genes.

FIG. 32A and FIG. 32B show an evaluation of the binary clonal expansion model. FIG. 32A shows a correlation between the predicted number of single cell clonal expansions compared to the true number of cells per clonotype per cell type. FIG. 32B. shows a ROC-AUC curve for the binary clonal expansion prediction.

FIG. 33 shows the predicted clonality compared to the ground truth coloniality, as measured by TCR data.

FIG. 34 shows the predicted percent of population expanded cells by cell population type.

FIG. 35 shows the percent of unique clones at the sample level and biological age of the sample.

### DETAILED DESCRIPTION

The disclosed invention demonstrates methods to predict single cell transcriptomic data from flow cytometry. Predicted single cell transcriptomic data can be used to generate single cell transcriptomic profiles for subjects. Transcriptomic profiles may comprise single cell transcriptomic data, emergent properties of single cell transcriptomic data such as gene signatures, cell trajectories, and/or transcriptional patterns, and emergent properties of single cell immune receptor profiling such as immune receptor diversity or predictions of clonal expansions. Transcriptomic profiles generated from subjects can be used for a wide range of applications, not limited to better diagnose an immune-related disease or disorder, monitor progression of an immune-related disease or disorder, or monitor a response to treatment of an immune-related disease or disorder in the subject.

The ability to predict even a fraction of this single cell transcriptomic data from flow cytometry would immediately expand the utility of flow cytometry data given the vastly improved statistical power and capture of diverse health and disease states. Predicting transcriptomic data would also substantially reduce the cost (>100× cheaper) of generating single-cell transcriptomic data for a subject. Methods directed to integrating immunophenotyping and single-cell transcriptomics data to overcome some of these challenges have been developed (see, e.g., Pepapi et al., (2023) “Integration of single-cell RNA-Seq and CyTOF data characterizes heterogeneity of rare cell subpopulations,” F1000Res 11:560). These methods marginally increase available immune cell profiling information from immunophenotyping techniques. However, these methods suffer from the same bottlenecks of single cell transcriptomic profiling such as expense, labor, limited sample sizes, and time. There are currently no technologies available to predict the detailed information provided by single-cell transcriptomics from cost effective and high throughput techniques like immunophenotyping with flow cytometry alone.

Machine learning models can be trained to recognize the relationship between single cell transcriptomic data and immunophenotyping data obtained from flow cytometry. The models can then be employed to output transcriptomics information from the immunophenotyping data. However, training these models requires training data comprising both single cell transcriptomic data and immune phenotyping data on the same cells. Current methods for flow cytometry and single cell transcriptomics cannot both be performed on the same cells. As a result, matched single cell transcriptomics data and immunophenotyping data cannot be collected and used to effectively train a model.

The development of methods to analyze both gene expression and immunophenotyping surface marker information from individual single cells, by sequencing, provide a unique resource for generating both types of data from the same cells. However, there is a technical challenge to align the marker data generated by sequencing and marker data generated by flow cytometry. These data types are both on a continuous scale but the signals and representations of cells lacking biological expression of each protein differ. Training a machine learning model on the unaligned data would decrease the ability of the model to recognize the connection between single cell gene expression and flow cytometry data once it is employed. Thus, these new methods also do not provide an effective way to generate the training data necessary to train a machine learning model to predict single cell transcriptomic data from flow cytometry alone.

The present application overcomes these challenges by splitting a sample into a first plurality and second plurality of cells, performing a sequencing method to analyze gene expression from the first plurality of cells and flow-cytometry on the second plurality of cells, and aligning the two data sets by generating or predicting pseudo-fluorescent marker data and/or pseudo-fluorescent cell classifications on the first plurality of cells. Specifically, generating pseudo-fluorescent marker data and/or pseudo-fluorescent cell classifications on the first plurality of cells solves the problem that transcriptomic data and flow cytometry data are traditionally collected on separate pluralities of cells. This is achieved by processing the data generated by sequencing of immunophenotyping surface markers into a format equivalent to that produced by flow cytometry (termed antibody-derived tags, or ADT), or predicting this information with a separate machine learning model trained on single cell transcriptomic data. Further, generating pseudo-fluorescent marker data and/or pseudo-fluorescent cell classifications on the first plurality of cells solves the problem that the immunophenotyping data collected on the same plurality of cells as the gene expression data is not on the same scale or in the same form as immunofluorescent data generated from flow cytometry. Training a machine learning model with pseudo-fluorescent marker data and/or pseudo-fluorescent cell classifications and transcriptomic on the same cells allows the trained models to effectively predict single cell transcriptomic data from flow cytometry alone.

The present application provides, in certain aspects, methods and systems for determining single cell expression and single cell transcriptomic profiles for a subject from only flow cytometry data. In some embodiments, the method comprises collecting flow cytometry data on a sample and providing the data to a machine learning model trained on single cell transcriptomic data and pseudo-fluorescent marker data and/or pseudo-fluorescent cell classifications generated on the same plurality of cells in order to predict transcriptomic information for that sample from just the flow cytometry information.

The machine learning models trained and used according to the current invention have flexible architectures based on the transcriptomic data that the models are trained to predict. The methods described herein may comprise automatic analysis of the distribution of gene expression for the single cells in the training data set in order to choose the most appropriate architecture for the machine learning model. The flexibility in the architecture may also comprise flexibility in the loss functions that are used to train the machine learning models. By adapting the model architecture and training parameters the methods disclosed herein can be easily applied to a wide range of transcriptomic predictions using flow cytometry data.

The machine learning models trained and used according to the current invention can be implemented in systems and pipelines for easy adaptation and workflow management. The systems and pipelines may consist of modules for preprocessing the data training the models predicting the transcriptomic values and evaluating the predicted results. The approach allows for implementing multiple variations of the system and pipelines for tuning performance of the predictions. In some embodiments, workflow managers such as Kedro, or other managers known in the art, are used. The modules can be configured by parameters that are specified by a user so that no changes to the underlying code are necessary and reproducibility is guaranteed within the system. The modules may be iterative to update and improve the predictions at each round automatically or with minimal human intervention.

### I. Definitions

Unless otherwise defined, all technical terms used herein have the same meaning as commonly understood by one of ordinary skill in the field to which this disclosure belongs.

As used in this specification and the appended claims, the singular forms “a” “an”, and “the” include plural references unless the context clearly indicates otherwise. Any reference to “or” herein is intended to encompass “and/or” unless otherwise stated, and encompasses any and all possible combinations of one or more of the associated listed items.

As used herein, the terms “includes, “including,” “comprises,” and/or “comprising” specify the presence of stated features, integers, steps, operations, elements, components, and/or units but do not preclude the presence or addition of one or more other features, integers, steps, operations, elements, components, units, and/or groups thereof.

Throughout this application, various parameter values may be presented in a range format. It should be understood that the description in range format is merely for convenience and brevity, and should not be construed as an inflexible limitation on the scope of the disclosure. Accordingly, the description of a range should be considered to have specifically disclosed all possible subranges as well as individual numerical values within that range, irrespective of whether a specific numerical value or specific sub-range is expressly stated. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 1.4, 2, 3, 3.6, 4, 5, 5.8, and 6. This applies regardless of the breadth of the range.

Numbers may be expressed herein as being “about” a particular value. Similarly, ranges may be expressed herein as from “about” one particular value and/or to “about” another particular value. The terms “about” and “approximately” shall generally mean an acceptable degree of error or variation for a given value or range of values, such as, for example, a degree of error or variation that is within 20 percent (%), within 15%, within 10%, or within 5% of a given value or range of values.

It should be recognized that use of ordinal terms such as “first” and “second” in the description of methods and systems disclosed herein does not by itself connote any priority, order of importance of one system component over another, or the temporal order in which acts of a method are performed, but are used merely as labels to distinguish, for example, one system component having a certain name from another system component having the same name but for the use of the ordinal term to distinguish the two system components.

Additionally, various implementations of the methods and systems set forth herein may be described in terms of exemplary block diagrams, process flow charts, and other illustrations. As will become apparent to one of ordinary skill in the art after reading this document, the various implementations set forth herein can be implemented without confinement to the illustrated examples. For example, block diagrams and their accompanying description should not be construed as mandating a particular architecture or configuration. Similarly, in exemplary process flow charts, some blocks are optionally combined, the order of some blocks is optionally changed, and some blocks are optionally omitted. In some implementations, additional steps may be performed in combination with the exemplary processes. Accordingly, the methods and systems as described and illustrated in greater detail below are exemplary by nature and, as such, should not be viewed as limiting.

As used herein, the terms “flow cytometry” and “flow cytometer” refer to a technique and instrument, respectively, for performing flow cytometry where the instrument is configured to capture emission of fluorescent molecules using arrays of highly sensitive light detectors, thereby enabling the capture of highly multiplexed fluorescence intensity data sets. This includes all variants of flow cytometry and mass cytometry technology, including but not limited to conventional flow cytometry and full spectrum flow cytometry.

As used herein, the term “immunophenotyping panel” refers to a panel of antibodies (e.g., fluorescently-labeled antibodies) that are used to identify cells based on the types of antigens or markers (e.g., cell surface receptor proteins) present on the surface of the cells.

As used herein, the term “gene” refers to the non-coding and coding regions of DNA that result in production of a given mRNA transcript and protein or protein subunit.

As used here, the term “gene expression” refers to a measurement related to the frequency of mRNA molecules derived from the coding region of a given gene.

As used herein, “single cell gene expression” refers to expression level for genes at a single cell level and may be expressed as mRNA transcript counts, normalized mRNA transcript counts, or through any other measure known in the art. Single cell gene expression data and single cell transcriptomic data may be used interchangeably or single cell transcriptomic data may refer to broader properties related to expression level of a gene. For example, single cell transcriptomics additionally encompasses the identity and precise sequences of immune receptor genes e.g. T cell receptor and B cell receptor genes which are often measured and analyzed alongside mRNA transcript measurements.

The section headings used herein are for organizational purposes only and are not to be construed as limiting the subject matter described. The description is presented to enable one of ordinary skill in the art to make and use the invention, and is provided in the context of a patent application and its requirements.

### II. Methods for Determining Single Cell Transcriptomic Data and Generating a Single Cell Transcriptomic Profile for a Subject from Flow Cytometry

### A. Generating a Single Cell Transcriptomic Profile

In some embodiments, disclosed herein are methods to train a machine learning model to generate a predicted single cell transcriptomic value. In some embodiments, the trained machine learning model may be deployed in methods to determine a predicted single cell transcriptomic value from flow cytometry data. In some embodiments, the trained machine learning model may be deployed to generate a single cell transcriptomic profile for a subject. In some embodiments, the single cell transcriptomic profile for a subject comprises a plurality of predicted single cell transcriptomic values.

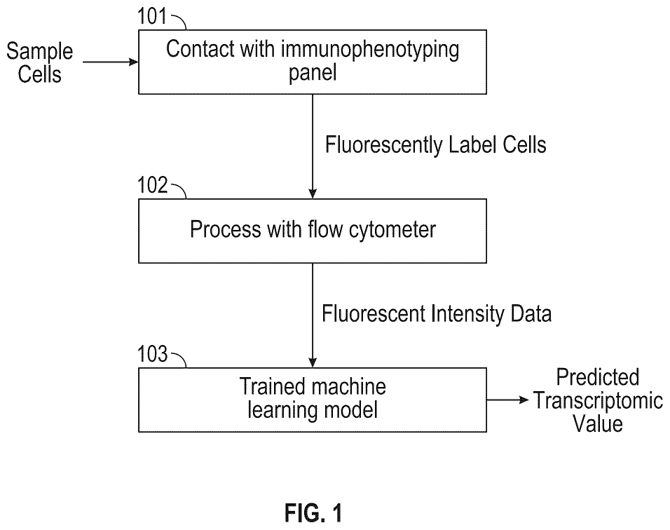

FIG. 1. provides a non-limiting example of a method disclosed herein. Sample cells are contacted with an immunophenotyping panel, 101, to generate fluorescently labeled cells. The fluorescently labeled cells are processed with a flow cytometer, 102, to generate fluorescent intensity data. The fluorescent intensity data is input into a trained machine learning model, 103, that has been trained to output a predicted transcriptomic value or profile.

In some embodiments, the first aliquot of a sample comprises sample cells. In some embodiments, the sample cells comprises between about 5,000 and about 19,000, between about 5,000 and about 18,000, between about 5,000 and about 17,000, between about 5,000 and about 16,000, between about 5,000 and about 15,000, between about 5,000 and about 14,000, between about 5,000 and about 13,000, between about 5,000 and about 12,000, between about 5,000 and about 10,000, between about 5,000 and about 9,000, between about 5,000 and about 8,000, between about 5,000 and about 7,000, or between about 5,000 and about 6,000 cells. In some embodiments, the sample cells comprises between about 6,000 and about 20,000, between about 7,000 and about 20,000, between about 8,000 and about 20,000, between about 9,000 and about 20,000, between about 10,000 and about 20,000, between about 11,000 and about 20,000, between about 12,000 and about 20,000, between about 13,000 and about 20,000, between about 14,000 and about 20,000, between about 15,000 and about 20,000, between about 16,000 and about 20,000, between about 17,000 and about 20,000, between about 18,000 and about 20,000, or between about 19,000 and about 20,000 cells.

In some embodiments, the subject may be a human. In some embodiments, the subject may be a member of a biological research study. In some embodiments, the subject may be a patient with a diagnosis of immune-related diseases and disorders, autoimmune diseases, or cancer. In some embodiments, the subject may be a patient with an infectious disease.

In some embodiments, the single cell transcriptomic profile may comprise a single cell transcriptomic value for at least about 100, about 1,000, about 5,000, about 10,000 or about 20,000 genes. In some embodiments, the single cell transcriptomic profile may comprise transcriptomic values for characterizing a group of cells. In some embodiments, the single cell transcriptomic profile may comprise immune receptor values for characterizing a group of cells.

In some embodiments, the single cell transcriptomic profile may comprise emergent properties of single cell transcriptomic data. In some embodiments, the emergent properties may be relevant to groups of cells in a sample. In some embodiments, the emergent properties may be gene signatures, cell trajectories, or transcriptional patterns.

In some embodiments, the single cell transcriptomic profile may comprise emergent properties of single cell immune receptor profiling. In some embodiments, the emergent properties may be relevant to groups of cells in a sample. In some embodiments, the emergent properties may be immune receptor diversity measurements or predictions of clonal expansions.

In some embodiments, the methods comprise collecting at least a first aliquot of a sample from a subject with at least a first immunophenotyping panel to fluorescently label cells within the sample. In some embodiments, the immunophenotyping panel comprises a panel of fluorescently labeled antibodies direct to cell surface proteins associated with antigen presenting cells. In some embodiments, the immunophenotyping panel comprises at least about 5, at least about 10, at least about 15, at least about 20, at least about 30, at least about 40, at least about 50, at least about 60, at least about 70, at least about 80, at least about 90 or at least about 100 fluorescently labeled antibodies. In some embodiments, the immunophenotyping panel comprises between about 5 and about 100, between about 10 and about 90, between about 20 and about 80, between about 30 and about 70, or between about 40 and about 60 fluorescently labeled antibodies.

In some embodiments, the sample from the subject may be whole blood. In some embodiments, the sample from the subject may comprise a whole blood sample, a buffy coat sample, a cell suspension, or peripheral blood mononuclear cell (PBMC) samples. In some embodiments, the sample from the subject may be immune cells that have been extracted from whole blood. In some embodiments, the sample may be cryopreserved.

In some embodiments, the methods comprise processing the fluorescently-labeled cells using flow cytometry, as described herein, to generate fluorescent intensity data, or data derived therefrom for a plurality of the fluorescently-labeled cells. In some embodiments, the flow cytometry is performed with a flow cytometer configured for at least about 5, at least about 10, at least about 15, at least about 20, at least about 30, at least about 40, at least about 50, at least about 60, at least about 70, at least about 80, at least about 90 or at least about 100 fluorescent detection channels. In some embodiments, the flow cytometry is performed with a flow cytometer configured for between about 5 and about 100, between about 10 and about 90, between about 20 and about 80, between about 30 and about 70, or between about 40 and about 60 fluorescent detection channels. In some embodiments, full spectrum flow cytometry is used.

In some embodiments, the methods comprise providing at least a subset of the fluorescent intensity data or data derived therefrom to a trained matching learning model. In some embodiments, the trained machine learning model will output a predicted single cell transcriptomic value for the sample. In some embodiments, the predicted single cell transcriptomic values will be related to single cell immune receptor sequences (e.g. predicted clonal expansion statuses). In some embodiments, the trained machine learning model will output one or more single cell transcriptomic values (e.g. single cell transcriptomic qualifications) for about 20,000 genes, about 10,000 genes, about 5,000 genes, about 2,000 genes, about 1,000 genes, about 500 genes, about 200 genes, or about 100 genes. In some embodiments, the trained machine learning model will output single cell transcriptomic values for highly variable genes. In some embodiments, the trained machine learning model will output one or more predicted single cell transcriptomic values for cell type related genes. In some embodiments, the single cell transcriptomic values can be combined to generate a single cell transcriptomic profile for the subject. In some embodiments, the single cell transcriptomic profile will comprise broad gene expression signatures applicable to groups of single cells.

In some embodiments, the method comprises determining predicted transcriptomic values for a group of cells. In some embodiments, the methods comprise providing at least a subset of the fluorescent intensity data or data derived therefrom to a trained matching learning model. In some embodiments, the trained machine learning model will output a predicted transcriptomic value for the sample. In some embodiments, the predicted single cell transcriptomic values will be related to single cell immune receptor sequences (e.g. predicted clonal expansion statuses). In some embodiments, the trained machine learning model will output broad transcriptomic signatures applicable to groups of single cells. In some embodiments, broad transcriptomic signatures applicable to groups of single cells can be combined to generate a single cell transcriptomic profile for the subject.

In some embodiments, the methods comprise determining a predicted transcriptomic value (e.g. predicted transcriptomic quantification) for a plurality of genes. In some embodiments, the methods comprise generating a single cell transcriptomic profile comprising predicted transcriptomic values (e.g. predicted transcriptomic quantification) for a plurality of genes. In some embodiments, the plurality of genes comprises highly expressed genes. In some embodiments, the plurality of genes comprises highly variable genes. In some embodiments, the plurality of genes comprises cell type related genes. In some embodiments, the plurality of genes comprises genes with non-negligible gene expression values. In some embodiments, the plurality of genes comprises a biologically curated gene set. The biologically curated gene set may be related to genes expressed in a cell type of interest. The biologically curated gene set may be related to marker genes (i.e. genes with high expression) for clusters of cell types. In some embodiments, the plurality of genes comprise all genes measured using a method for quantifying gene expression at a single cell level as described herein. In some embodiments, the plurality of genes comprise the plurality of genes used for training the machine learning model. As a non-limiting example, a machine learning model may be trained as described herein using genes that were classified as highly expressed in the training data and thus the machine learning model may be used to predict a transcriptomic quantification for each gene in the training set.

The disclosed methods and systems for determining a predicted single cell transcriptomic value and generating a single cell transcriptomic profile for a subject may be used for a variety of biological research and clinical diagnostic applications. Examples include, but are not limited to, diagnosis of immune-related diseases and disorders, diagnosis of autoimmune diseases and cancer, diagnosis of and/or identification of individual-specific response to infectious diseases, the prediction of response to treatment either prior to or during treatment, and the characterization of donors and products in cell therapy manufacturing. The disclosed methods and systems for determining a predicted single cell transcriptomic value and generating a single cell transcriptomic profile for a subject may be used for new and retrospective profiling. Samples that have been cryopreserved or newly collected can be used. The samples can also be collected at multiple time points in order to profile a sample at multiple time points over a clinical time course.

The methods described here in for predicting a single cell transcriptomic value and generating single cell transcriptomic profiles for a subject from flow cytometry can be used to augment clinical outcome prediction for the subject using flow cytometry. In some embodiments, the predicted single cell transcriptomic values and/or predicted single cell transcriptomic profiles can be used to train or used as complementary inputs for additional machine learning models to predict clinical outcomes. In some embodiments, the additional models may have been trained using fluorescent intensity data and or immune profile data. In some embodiments, the immune profile data may have been generated using ML-based cell classification according to the methods described in U.S. Ser. No. 18/353,022, and corresponding U.S. Patent publication US2024-0192210-A1.

In some embodiments, the clinical outcomes may relate to response to a solid organ transplantation. In some embodiments, the additional machine learning models may comprise the models described in U.S. 63/657,703, hereby incorporated by reference. In some embodiments, the clinical outcomes may relate to success for donation or receipt of a hematopoietic stem cell transplant (HSCT). In some embodiments, the additional machine learning models may comprise the models described in U.S. 63/712,272, hereby incorporated by reference. In some embodiments, the clinical outcomes may relate to response to response to a cancer vaccine. In some embodiments, the additional machine learning models may comprise the models described in U.S. 63/740,149, hereby incorporated by reference. In some embodiments, the clinical outcomes may relate to response to response to a cancer therapy, such as but not limited to an immune checkpoint inhibitor. In some embodiments, the additional machine learning models may comprise the models described in U.S. 63/761,725, hereby incorporated by reference.

Incorporating the predicted single cell transcriptomic data into any of the additional machine learning models may maximize the extraction of potential predictive information from flow cytometry data and thus will yield improved clinical outcome prediction accuracy. Single cell gene expression prediction from fluorescence data using the methods described herein, may provide access to latent information within flow cytometry samples that could otherwise be hidden when summary cell classification information is derived and used for model training. Predicted single cell transcriptomic data (either single cell transcriptomic quantifications and/or single cell immune cell receptor profile information derived therefrom) can augment the source Flow cytometry data in clinical outcome prediction models to supply additional layers of sample information and thus enhance the potential predictive accuracy.

The methods described herein for determining single cell transcriptomic values and generating a single cell transcriptomic profile for a subject can be used to complement single cell transcriptomic data collected using RNA sequencing. Single cell RNA sequencing performed on blood cells is limited in the number of cells that can be profiled. The limit on profiling of the cells creates sampling errors given the complexity of cell populations in states circulating in the human immune system. In comparison, flow cytometry can be performed on more cells from a human blood sample and less drop out of sample is expected. By complementing single cell RNA sequencing data for blood cells with predicted with predicted single cell transcriptomic values predicted from flow cytometry data. The complexity of the human immune system can be analyzed with higher complexity and thus lower amount of sampling error.

In some embodiments, complementing single cell RNA sequencing comprises single cell RNA sequencing upscaling. The predicted single cell transcriptomic values harness the statistical robustness of flow cytometry-scale cell profiling (1-2 orders of magnitude more cells) to “upscale” bona fide single cell RNA sequencing data. to sufficient cell numbers for downstream statistical analysis. In some embodiments, the predicted single cell transcriptome link values can be used as bona fide single cell RNA sequencing data when single cell RNA sequencing and flow cytometry are performed on match samples. The single cell RNA sequencing data may provide ground truth, single cell expression data, which provides a reference for the machine learning model to predict additional single cell transcriptomic values from the flow cytometry data.

### B. Machine Learning Models

In some embodiments, the disclosed invention comprises a machine learning (algorithm) model that may be trained to generate a predicted single cell transcriptomic value, profile, and/or emergent characteristics of single cell transcriptomics data, including but not limited to immune receptor profile diversity, cell trajectory information and gene signatures. As used herein machine learning model may alternatively be referred to as a machine learning algorithm. In some embodiments, the training is based on the single cell transcriptomic data for at least a subset of the first plurality of cells and the pseudo-fluorescent data for the at least a subset of the first plurality of cells. In some embodiments, the model is trained using cells in the first plurality of cells, wherein each cell is associated with single cell transcriptomic data and pseudo-fluorescent data. In some embodiments, the trainable features are in the pseudo-fluorescent data. In some embodiments, the trainable features are pseudo-fluorescent cell classifications, pseudo-fluorescent markers, or both for each of the single cells. In some embodiments, the target variables are in the single cell transcriptomic data. In some embodiments, the target variables are the transcriptomic values (e.g. single cell transcriptomic quantification) for a gene in each of the single cells and/or clonal expansion statuses for the single cells. In some embodiments, the target variables are properties emergent from single cell transcriptomic or immune receptor profiling data, such as population heterogeneity and gene signatures.

In some embodiments, the training is based on the gene expression data and/or other single cell transcriptomics data for at least a subset of the first plurality of cells and the pseudo-fluorescent cell classification data for the at least a plurality of the first cells. In some embodiments, the model is trained using cells in the first plurality of cells, wherein each cell is associated with single cell transcriptomic data and a pseudo-fluorescent cell classification. In some embodiments, the trainable features are pseudo-fluorescent cell classifications for each of the single cells. In some embodiments, the target variables are the single cell transcriptomic data (e.g. single cell transcriptomic quantifications and/or clonal expansion statutes). In some embodiments, the target variables are the transcriptomic values (e.g. single cell transcriptomic qualification) for a gene or a plurality of genes in each of the single cells. In some embodiments, the target variables are properties emergent from single cell transcriptomic or immune receptor profiling data, such as population heterogeneity and gene signatures.

In some embodiments, the training is based on the single cell gene expression data and/or other single cell transcriptomics data for at least a subset of the first plurality of cells and the pseudo-fluorescent data for the at least a plurality of the first cells. In some embodiments, the model is trained using cells in the first plurality of cells, wherein each cell is associated with single cell transcriptomic data and a pseudo-fluorescent data. In some embodiments, the trainable features are pseudo-fluorescent data for each of the single cells. In some embodiments, the trainable features are pseudo-fluorescent data for all protein markers in each of the single cells. In some embodiments, the target variables are the single cell transcriptomic data (e.g. single cell transcriptomic quantifications and/or clonal expansion statutes). In some embodiments, the target variables are the gene expression values for a gene or plurality of genes in each of the single cells. In some embodiments, the target variables are properties emergent from single cell transcriptomic or immune receptor profiling data, such as population heterogeneity and gene signatures.

In some embodiments, the training is based on the gene expression data and/or other single cell transcriptomics data for at least a subset of the first plurality of cells, the pseudo-fluorescent data for the at least a plurality of the first cells, and the pseudo-fluorescent cell classification data for the at least a subset of the first plurality of cells. In some embodiments, the model is trained using cells in the first plurality of cells, wherein each cell is associated with transcriptomic data, pseudo-fluorescent data, and a pseudo-fluorescent cell classification. In some embodiments, the trainable features are pseudo-fluorescent data and the pseudo-fluorescent cell classification for each of the single cells. In some embodiments, the trainable features are pseudo-fluorescent data for all protein markers in each of the single cells and the accompanying pseudo-fluorescent cell classification. In some embodiments, the target variables are the gene expression data (e.g. single cell transcriptomic quantifications and/or clonal expansion statutes). In some embodiments, the target variables are the transcriptomic values for a gene or plurality of genes in each of the single cells. In some embodiments, the target variables are properties emergent from single cell transcriptomic or immune receptor profiling data, such as population heterogeneity and gene signatures.

In some embodiments, the machine learning model may be a neural network. In some embodiments, the machine learning model may be a convolutional neural network. In some embodiments, the machine learning model may be a multilayer neural network. In some embodiments, the neural network may comprise a single input layer, zero or more fully or partially connected layers, and a fully or partially connected single output layer of 100 to a plurality of nodes. In some embodiments, the machine learning model may be a bayesian neural network.

In some embodiments, the neural networks may be fully connected deep networks with a latent layer and empirically determined number and size of layers. One model per gene may be trained or one model may be trained for predicting all genes.

In some embodiments, the neural network may be implemented using JAX, tensorflow, or pytorch. In some embodiments, the output layer comprises predicted gene expression values for each single cell in an input. In some embodiments, the gene expression values may comprise a value for the entire transcriptome, highly variable genes, or cell type-relevant genes. In some embodiments, the model hyperparameters are tunable per a specific application of model training.

In some embodiments, the machine learning model may be a hybrid classifier regression neural network. In some embodiments, the machine learning model may be a hybrid classifier regression multilayer neural network. In some embodiments, the hybrid classifier regression neural network may be trained to output a categorical and a continuous output. For example, the machine learning model may output a binary classification of whether a gene is expressed as well as a continuous output predicting gene expression. In some embodiments, a predicted transcriptomic value as described herein may be a combination of the categorical output and continuous output. As a non-limiting example, the categorical output may be a 0 or 1 and may be multiplied by the continuous output.

In some embodiments, a variational auto encoder followed by the neural network may be used to reduce the dimensionality of the gene expression using an auto encoder and then predicting the latent representation using the decoder component. In some embodiments, graph neural networks may be used to incorporate gene pathway info and/or cell identity relationships into the graph thereby improving the prediction of the transcriptomic values In each cell from the graph activations.

In some embodiments, the machine learning model may be organized in a cascading hierarchical tree structure comprising a plurality of nodes. In some embodiments, each node may be an individual machine learning model. In some embodiments, each individual machine learning model may comprise a neural network. In some embodiments, each individual machine learning model may comprise a gradient boosting tree model. In some embodiments, the gradient boosted tree model is XGBoost.

In some embodiments, the machine learning model comprises a hybrid classifier regression multilayer neural network. In some embodiments, the machine learning model comprises a regression multilayer neural network. In some embodiments, the machine learning model comprises a tweedie regression. A tweedie distribution regression may be used for distributions with a large number of zeros and/or right skewed distributions. In some embodiments, the machine learning model comprises a hybrid mean standard error (MSE)/Tweedie neural network. In some embodiments, the machine learning model comprises a gradient descent model.

As described herein, the machine learning models may have flexible architectures. In some embodiments, a model architecture can be chosen based on the distribution of the single cell transcriptomic data used for training the model. In some embodiments, the methods described to herein comprise selecting a machine learning model architecture from a group of machine learning model architectures based on a distribution of the single cell transcriptomic data. For example, the machine learning model architecture may be selected based on the distribution of single cell gene transcriptomic values (e.g. single cell transcriptomic quantifications) for the plurality of genes used in training. In some embodiments, the machine learning model architecture may be selected from any of the machine learning model architectures described herein.

The selected machine learning model architecture may be selected based on the desired prediction. A binary classifier, a simple logistic regression, a tree based ensemble model such as gradient boosted trees and random forest classification and/or feed forward neural networks (e.g. multi-layer perceptron, partially-connected deep neural networks with a single-node output layer with a sigmoid activation function) or transformer based models may be used for predicting binary cell level annotations, such as predicted expansion statuses

In some embodiments, the machine learning model comprises a transformer based model. In some embodiments, protein marker intensities and gene expression can be converted into embeddings and gene pathway information or other publicly available information about the cells can be added during training of the transformer to improve predictions.

In some embodiments, modifications can be employed to improve prediction accuracy using biologically informed guidance. Non-limiting examples of modifications that can be used for incorporating biologically informed guidance Include cell type weighted networks cell type information and gene pathway information. In a cell type weighted network, loss functions in training can be evaluated on a set of mutually exclusive, biologically-informed population classification memberships (rather than bulk cells) to weight the loss equally across transcriptionally distinct cell types. Rarer cell types can also/alternatively be up sampled to minimize bias of the network to abundant cell types. The cell type (e.g. scRNAseq-defined clusters; FSFC-defined population memberships) can be encoded in the input of the training data to help the model to capture the cell type distinctions. Gene pathway information from publicly available databases can be included as model input for certain architectures e.g. graph neural networks.

As described herein, training the machine learning models may comprise minimizing a loss function. The loss function may be flexible like the machine learning model architecture and may be selected based on the distribution of the single cell transcriptomic data used for training the model. In some embodiments, the methods comprise selecting one or more loss functions for training the machine learning model. In some embodiments, the one or more loss functions may be selected from a group consisting of a log likelihood loss based on a tweedie distribution, negative log likelihood loss, mean squared error loss, mean absolute error (MAE), and cross entropy loss.

Given the technical challenges in predicting zero-inflated continuous single cell transcriptomic quantification, different loss functions can be employed alone or together to improve performance of predicting single cell transcriptomic values for different gene sets with different distributions (e.g. highly expressed, highly variable, non-negligible).

Regression loss functions may be employed to optimize prediction of single cell transcriptomic quantifications. Mean squared error loss may be suitable for continuous predictions for highly expressed genes. Mean absolute error loss may be used may be suitable for continuous predictions for highly variable genes. Negative log likelihood loss may be suitable for continuous predictions for poorly expressed genes with a high proportion of 0 values (i.e. a zero-inflated distribution).

The sparsity of single cell gene expression data can be addressed by including binary classification of genes as 0 or non-0, in combination with regression models as describe herein: Loss functions that may be used for binary classifications may include cross entropy loss, focus loss, or Huber loss. In some embodiments, weighted cross entropy loss may be used to focus on samples with greater uncertainty (i.e. close to the decision boundary)

As described herein, the loss functions are suited to different gene expression distributions and can be flexibly implemented, such that the model can incorporate different functions for different partitions of the training data. For example, a hybrid neural network approach can employ a binary classifier using cross entropy loss to predict 0 vs. non-0 values in parallel with continuous prediction using mean square error loss.

The loss functions calculate the mean loss for each of the outputs in each model and the training performs a minimization of the combined losses. It is contemplated that the loss may be biased towards genes with low variance if they form the large majority of the plurality of genes used to train the model In some embodiments, the losses can be weighted by the variance of each gene In order to prioritize high variable and high expressed genes.

In some embodiments, each layer of a neural network comprises a number of nodes (or perceptrons). In some embodiments, a node receives input that comes either directly from the input data (e.g., pseudo-fluorescent data) or the output of nodes in previous layers, and performs a specific operation, e.g., a summation operation. In some cases, a connection from an input to a node is associated with a weight (or weighting factor). In some cases, the node may, for example, sum up the products of all pairs of inputs from a previous layer and their associated weights. In some cases, the weighted sum is offset with a bias, b. In some cases, the output of a node may be gated using a threshold or activation function, f, which may be a linear or non-linear function. The activation function may be, for example, a rectified linear unit (ReLU) activation function or other function such as a saturating hyperbolic tangent, identity, binary step, logistic, arc Tan, softsign, parametric rectified linear unit, exponential linear unit, softPlus, bent identity, softExponential, Sinusoid, Sine, Gaussian, or sigmoid function, or any combination thereof.

In some embodiments, the weighting factors, bias values, and threshold values, or other computational parameters of the neural network, can be “taught” or “learned” in a training phase using one or more sets of training data. For example, the parameters may be trained using the input data from a training data set and a gradient descent or backward propagation method so that the output value(s) (e.g., gene expression values) that the neural network predicts are consistent with the examples included in the training data set. The adjustable parameters of the model may be obtained using, e.g., a back propagation neural network training process.

In some embodiments, the plurality of nodes (i.e., the number of individual machine learning models in the ensemble) comprises at least 1000, 1200, 1400, 1600, 1800, 2000, 2200, or 2400 nodes. In some instances, the number of individual machine learning models in the ensembled machine learning model is equal to a few distinct genes in the single cell transcriptomic data. In some instances, the number of individual machine learning models in the ensemble machine learning model is equal to a number of distinct classifications in the pseudo-fluorescent cell classification.

In some embodiments, the machine learning model may suffer from overfitting. In some embodiments, hard-parameter sharing may be used to help with mitigation of loss inherent to overfitting models. In some embodiments, hard-parameter sharing may be implementing a deep neural network in which the deeper hidden layers are shared between all tasks (which learn and simultaneously reduce the dimensionality of the information contained in the features), while each target (or biologically-informed group of targets) has dedicated output layers in the network architecture which serve to pseudo-independently predict the transcriptomic values from the encoded information of the hidden layers.

In some embodiments, the complex interdependencies and correlations between marker expression levels in the pseudo-fluorescent data and transcriptomic data. In some embodiments, a convolutional approach may be employed to capture the inter dependencies. Convolutional neural networks have been shown to be a powerful approach in image analysis, in which pixel-to-pixel correlations are inherent and form shapes that constitute the meaning of the image. CNNs have also been adapted to non-image data, in which the order of the “pixels” (or features and samples in ML nomenclature) do not contain useful information. These approaches require the network to be made agnostic to the order of the input data.

In some embodiments, some protein markers captured in the pseudo-fluorescent data may correlate more strongly with gene expression than others. For example, it may be that markers A and B have strongest correlations to gene C. In some embodiments, attention mechanisms can be used to predict a specific gene by focusing only on specific markers. In some embodiments, attention mechanisms are used to weight the neural network model marker inputs such that the model can dynamically learn which markers contribute to which genes and weight those input features accordingly. In some embodiments, higher weights are given to markers more heavily associated with a target gene and lower weight to those markers with lower gene associations.

In some embodiments, additional model architectures may be used. In some embodiments, a “one model per gene” ensemble approach where each model may be a decision tree based model, a neural network of any architecture, or any other machine learning model known in the art that allows the hyperparameters to be modified and tuned between transcriptomic predictors for each gene.

In some embodiments, a multilevel Mixture of Experts approach may be employed where a subset of previously trained models can be used to vote, and a second later discriminatory machine learning model makes the final prediction.

### C. Generating Training Data

In some embodiments, disclosed herein are methods to train a machine learning model (e.g. model) to generate a predicted single cell transcriptomic value. In some embodiments, the machine learning model is trained to generate a single cell transcriptomic value from flow cytometry data. In some embodiments, the machine learning model is trained to generate a single cell transcriptomic value from only flow cytometry data. As described herein, the method comprise providing only at least a subset of fluorescent intensity data, or data derived therefrom for the plurality of fluorescently labelled cells as input into a machine learning model. Providing only at least at least a subset of fluorescent intensity data, or data derived therefrom for the plurality of fluorescently labelled cells refers to providing a least a subset of fluorescent intensity data, or data derived therefrom for the plurality of fluorescently labelled cells without providing single cell transcriptomic data for a plurality of cells from the sample. In some embodiments, the machine learning model is trained to generate a single cell transcriptomic profile for a subject. In some embodiments, the machine learning model is trained to generate a single cell transcriptomic immune receptor profile for a subject.

### 1. Collecting a Population of Cells

In some embodiments, training a machine learning model to generate a predicted single cell transcriptomic value, values, or profile comprises collecting a population of cells comprising a first plurality of cells and a second plurality of cells. In some embodiments, the population of cells may be whole blood. In some embodiments, the population of cells may comprise a blood sample, a buffy coat sample, a cell suspension, or peripheral blood mononuclear cell (PBMC) samples. In some embodiments, the population of cells may be immune cells, e.g. T cells, that have been extracted from whole blood. In some embodiments, the population of cells may be cryopreserved.

In some embodiments, the population of cells may be collected from a single individual. In some embodiments, the population of cells may be collected from a plurality of individuals. In some embodiments, the population of cells may comprise between about 100,000 and about 1,000,000 cells. In some embodiments, the population of cells may comprise between about 100,000 and about 200,000, between about 100,000 and about 300,000, between about 100,000 and about 400,000, between about 100,000 and about 500,000, between about 100,000 and about 600,000, between about 100,000 and about 700,000, between about 100,000 and about 800,000, between about 100,000 and about 900,000 cells. In some embodiments, the population of cells may comprise between about 200,000 and about 1,000,000, between about 300,000 and about 1,000,000, between about 300,000 and about 1,000,000, between about 400,000 and about 1,000,000, between about 500,000 and about 1,000,000, between about 600,000 and about 1,000,000, between about 700,000 and about 1,000,000, between about 800,000 and about 1,000,000, or between about 900,000 and about 1,000,000. In some embodiments, the population of cells may comprise greater than about 1,000,000 cells.

In some embodiments, the population of cells are split into a first plurality of cells and a second plurality of cells. In some embodiments, the first plurality of cells and the second plurality of cells originate from the same individual. In some embodiments, the first plurality of cells and the second plurality of cells originate from the same sample or collection.

In some embodiments, the first plurality of cells may comprise between about 5,000 and 20,000 cells. In some embodiments, the first plurality of cells may comprise between about 5,000 and about 19,000, between about 5,000 and about 18,000, between about 5,000 and about 17,000, between about 5,000 and about 16,000, between about 5,000 and about 15,000, between about 5,000 and about 14,000, between about 5,000 and about 13,000, between about 5,000 and about 12,000, between about 5,000 and about 10,000, between about 5,000 and about 9,000, between about 5,000 and about 8,000, between about 5,000 and about 7,000, or between about 5,000 and about 6,000 cells. In some embodiments, the first plurality of cells may comprise between about 6,000 and about 20,000, between about 7,000 and about 20,000, between about 8,000 and about 20,000, between about 9,000 and about 20,000, between about 10,000 and about 20,000, between about 11,000 and about 20,000, between about 12,000 and about 20,000, between about 13,000 and about 20,000, between about 14,000 and about 20,000, between about 15,000 and about 20,000, between about 16,000 and about 20,000, between about 17,000 and about 20,000, between about 18,000 and about 20,000, or between about 19,000 and about 20,000 cells. In some embodiments, the first plurality of cells may comprise more than about 20,000 cells.

In some embodiments, the second plurality of cells may comprise between about 100,000 and about 1,000,000 cells. In some embodiments, the second plurality of cells may comprise between about 100,000 and about 200,000, between about 100,000 and about 300,000, between about 100,000 and about 400,000, between about 100,000 and about 500,000, between about 100,000 and about 600,000, between about 100,000 and about 700,000, between about 100,000 and about 800,000, between about 100,000 and about 900,000 cells. In some embodiments, the second plurality of cells may comprise between about 200,000 and about 1,000,000, between about 300,000 and about 1,000,000, between about 300,000 and about 1,000,000, between about 400,000 and about 1,000,000, between about 500,000 and about 1,000,000, between about 600,000 and about 1,000,000, between about 700,000 and about 1,000,000, between about 800,000 and about 1,000,000, or between about 900,000 and about 1,000,000. In some embodiments, the second plurality of cells may comprise greater than about 1,000,000 cells.

### 2. Obtaining Transcriptomic Data and Protein Marker Data for Each Cell in the First Plurality of Cells

In some embodiments, training a machine learning model to generate a predicted single cell transcriptomic value comprises obtaining transcriptomic data and protein marker data from each cell in the first plurality of cells. In some embodiments, single cell transcriptomic data and protein marker data are simultaneously collected using a method for characterizing a cell. In some embodiments, the method allows for simultaneous detection of a plurality of protein markers and gene expression values for a plurality of genes at a single cell level. In some embodiments, the method is Cellular Indexing of Transcriptomes and Epitopes by Sequencing (CITE-seq) (Stoeckius et al, Nature Methods, 14, 865-866 (2017)). In some embodiments, the protein marker data may comprise an antibody-derived tag (ADT) marker value. In some embodiments, the Single cell transcriptomics data comprises clonal expansion statuses. In some embodiments, the method comprises immune receptor profiling (e.g. TCR and/or BCR sequencing). In some embodiments, the method comprises Chromium Next Gen single cell 5′ sequencing, performed with feature barcoding technology for cell surface protein and immune receptor mapping.

In some embodiments, obtaining gene expression data and protein marker data from each cell in the first plurality of cells comprises preparing data from ADTs. In some embodiments, the ADT comprises an antibody directed against a cell surface protein of interest, wherein the antibody is conjugated to an oligonucleotide barcode. In some embodiments, the oligonucleotide barcode allows for barcoding the antibody. In some embodiments, the method further comprises contacting the ADTs with the cells in the first plurality of cells. In some embodiments, contacting the ADTs with the cells comprises mixing the antibodies with the cells, wherein multiple antibodies can bind to multiple cells in the first plurality of cells. In some embodiments, this takes place prior to application of the cells to a microfluidic device. In some embodiments, contacting the ADTs with the cells in the first plurality of cells allows the ADTs to bind with antigen epitopes on the surface of immune cells. In some embodiments, binding ADTs with antigen epitopes allows for measuring protein expression on the surface of the cell.

In some embodiments, the contacting of the ADTs with the cells in the first plurality of cells comprises mixing the cells with antibodies in a tube, washing the cells, then applying the cells to a microfluidic device with the antibodies attached the surface epitopes of the cells.

In some embodiments, training a machine learning model to generate a predicted single cell transcriptomic value comprises obtaining single cell transcriptomic data and protein marker data from each cell in the first plurality of cells. In some embodiments, the transcriptomic data and protein marker data are profiled with CITE-seq, Chromium Next Gen single cell 5′ sequencing, performed with feature barcoding technology for cell surface protein and immune receptor mapping or an equivalent method to generate single cell transcriptomic data and protein marker data simultaneously.

In some embodiments, training a machine learning model to generate a predicted single cell transcriptomic value comprises obtaining single cell transcriptomic data and protein marker data from each cell in the first plurality of cells. In some embodiments, the transcriptomic data is generated by immune cell receptor profiling. In some embodiments, immune cell receptor profiling may be used to profile antigen receptor gene sequences. In some embodiments, the transcriptomic data is generated by any scRNAseq method known in the art.

In some embodiments, training a machine learning model to generate a predicted single cell transcriptomic value comprises obtaining single cell transcriptomic data and protein marker data from each cell in the first plurality of cells. In some embodiments, the protein marker data is generated by providing at least a subset of single cell transcriptomic data to a machine learning model configured to transform single cell transcriptomic data into protein marker data and output protein marker data.

In some embodiments, obtaining gene expression data and protein marker data from each cell in the first plurality of cells further comprises preparing one or multiple single cell RNA sequencing (scRNAseq) library/libraries. In some embodiments, the preparation comprises encapsulating the cells that have been contacted with the ADTs and DNA-barcoded microbeads within a droplet, lysing the cells in the droplet allowing for mRNAs and ADTs to hybridize to the DNA-barcoded microbead, generating cDNA from the hybridized molecules, and amplifying the cDNA for sequencing. In some embodiments, the DNA-barcodes hybridized to the microbeads comprise unique barcodes to tag individual cells and unique barcodes to tag individual molecules. In some embodiments, the scRNAseq library generation further comprises cDNA size selection. In some embodiments, the sRNA seq library preparation may be DropSeq (Macosko et al. Cell, 161(5), 1202-1214 (2015), 10× Genomics Chromium, or another large scale oligodT-based scRNA sequencing method known in the art. In some embodiments, the single cell transcriptomic data is generated using Chromium Next Gen single cell 5′ sequencing, performed with feature barcoding technology for cell surface protein and immune receptor mapping

In some embodiments, obtaining transcriptomic data and protein marker data, from each cell in the first plurality of cells further comprises performing next generation sequencing on the prepared scRNAseq libraries. In some embodiments, the preparation comprises encapsulating the cells and DNA-barcoded microbeads within a droplet, lysing the cells in the droplet allowing for mRNAs hybridize to the DNA-barcoded microbead, generating cDNA from the hybridized molecules, and amplifying the cDNA for sequencing. In some embodiments, the DNA-barcodes hybridized to the microbeads comprise unique barcodes to tag individual cells and unique barcodes to tag individual molecules. In some embodiments, the scRNAseq library generation further comprises cDNA size selection. In some embodiments, the sRNA seq library preparation may be DropSeq (Macosko et al. Cell, 161(5), 1202-1214 (2015), 10× Genomics Chromium, or another large scale oligodT-based scRNA sequencing method known in the art.

In some embodiments, scRNA sequencing generates single cell gene expression data. In some embodiments, scRNA sequencing generates single cell gene transcriptomic data. In some embodiments, scRNA sequencing generates single cell gene transcriptomic and protein marker values. In some embodiments, scRNAseq libraries may be pooled before sequencing. In some embodiments, the sequencing data generated using the scRNAseq library comprises data that can be aligned to a reference genome to generate mRNA counts, and ADT marker expression counts indexed by each cell in the first plurality of cells. In some embodiments, the sequencing data generated using the scRNAseq library comprises data that can be aligned to a reference genome to generate mRNA counts indexed by each cell in the first plurality of cells. In some embodiments, computational data analysis methods known in the art can be used to generate gene expression data and protein marker data from the mRNA counts and ADT marker expression counts. In some embodiments, computational data analysis methods known in the art can be used to generate gene expression data from the mRNA expression counts. In some embodiments, the computational data analysis methods comprise normalizing the counts. In some embodiments, the computational data analysis methods comprise imputing missing gene expression values (“technical dropouts”) using existing methods e.g. MAGIC. In some embodiments, computational data analysis methods known in the art can be used to identify paired immune receptor sequences and perform clonotype analysis from immune receptor profiling libraries. In some embodiments, data analysis may be performed using the CITE-seq-Count python package. In some embodiments, data analysis may be performed using the R packages CellRanger and Seurat.

Methods known in the art for processing sequencing data, such as single cell sequencing data may be used. Publicly available pipelines may be employed. In some embodiments, quality control may be performed to remove low quality data and verify data integrity using publicly available software packages. In some embodiments, methods may be used to standardize normalize and correct for batches in the data using methods known in the art.

In some embodiments, accommodations for memory limitations related to large data sets may be used for example to enables memory-efficient processing through representative subsampling for dimensionality reduction and clustering, alongside bit-packed compression of count matrices.

In some embodiments, dimensionality reduction on selected features may be performed to create low dimensional embeddings of the single cell transcriptomic data. These methods may include feature selection using variance stabilized transformation (VST), principal component analysis (PCA) of top variable features, or uniform manifold approximation and projection (UMAP) of top principal components (PCs). In some embodiments, the methods comprise identifying cellular clusters. Clustering may be performed using nearest neighbor graphs constructed from top PCs Or methods known in the art for identifying clusters using differential gene expression.

In some embodiments, the single cell transcriptomic data, or single cell transcriptomic data and protein marker data comprise data for about 4000-15000 cells in the first plurality of cells. In some embodiments, the first plurality of cells comprises about 4000-15000 cells. In some embodiments, the first plurality of cells comprises about 4000, about 5000, about 6000, about 7000, about 8000, about 9000, about 10000, about 110000, about 12000, about 13000 about 14000, or about 15000 cells.

In some embodiments, the single cell transcriptomic data collected for the first plurality of cells is single cell mRNA sequencing data. In some embodiments, the single cell mRNA sequencing data provides mRNA expression values for about 100, about 1,000, about 5000, about 10000, or about 20,000 genes in each cell in the first plurality of cells. In some embodiments, the single cell mRNA sequencing data provides mRNA expression values for between about 100 and 20,000 genes, about 1,000 and 20,000 genes, or about 5,000 and 20,000 genes in each cell in the first plurality of cells.

In some embodiments, the single cell transcriptomic data comprises single cell transcriptomic quantifications for a plurality of genes. In some embodiment, the plurality of genes comprises highly expressed genes. In some embodiments, the plurality of genes comprises highly variable genes. In some embodiments, the plurality of genes comprises cell type related genes. In some embodiments, the plurality of genes comprises genes with non-negligible gene expression values. In some embodiments, the plurality of genes comprises a biologically curated gene set. The biologically curated gene set may be related to genes expressed in a cell type of interest. The biologically curated gene set may be related to marker genes (i.e. genes with high expression) for clusters of cell types.

In some embodiments, the single cell transcriptomic data comprises clonal expansion statuses. In some embodiments, the clonal expansion statuses are generated from immune receptor profiling data. Immune receptor profiling data may be collected simultaneously to the other single cell transcriptomic data (single cell transcriptomic quantifications) and protein marker data. The methods for immune receptor profiling may be part of a CITE-seq protocol. In some embodiments, the methods for immune receptor profiling comprise Chromium Next Gen single cell 5′ sequencing, performed with feature barcoding technology for cell surface protein and immune receptor mapping. In some embodiments, the immune receptor profile data comprises TCR and/or BCR sequence data. TCR and/or BCR sequence data may comprise TCR and/or BCR sequences for cells. In some embodiments, the data comprise TCR sequences for T cells. In some embodiments, the data comprised BCR sequences for B cells. The unique TCR and BCR sequences for a sample may be recorded and can be used to annotate cells as non-expanded if the sequence is unique or expanded if the sequence is not unique in the sample. The expansion status may be expanded or non-expanded for each cell.

In some embodiments, various biological information may be derived from TCR and or BCR clonotype analysis. In some embodiments, the clonotype analysis information may be used alongside pseudo-fluorescence data for training the machine learning models described herein. The drive biological information may comprise cell level annotations of clonally expanded or non-expanded cells, or sample level diversity metrics to measure overall composition of TCR or BCR profiles tor an individual. In some embodiment sample level diversity metrics may be a Gini coefficient, a Shannon entropy and/or a Simpson index.

In some embodiments, the protein marker data comprise protein marker values for a plurality of surface marker proteins. In some embodiments, the protein marker values correspond to values for CD16, HLA-DR, CD56, CD4, CD14, TCRgd, CD3, CD8, CD45, CD38, PD1, CD5, CD19, CD319, CD138, CD10, CRTH2, CD303, IgM, IgD, CD337, CD24, CD40, CD141, CD267, CD69, CD27, CD11c, CD274, CD86, CD335, CD43, CD62L, CD123, CD1c, CD278, CD25, CD223, CD161, CD122, TCR Va7.2, TCR Va24-Ja18, CD45RO, CD95, CD127, CD366, KLRG1, CD28, CD45RA, CD31, CD57, CD39, TIGIT, CD103, TCR Vd2, TCR Vd1, CD194, CD183, CD197, CD196, CCR10, CD185, and TCRgd. In some embodiments, the protein marker data are generated from scRNAseq data. In some embodiments, protein marker values are measured directly during scRNA sequencing using CITE-seq technology or equivalent. In some embodiments, protein marker values are predicted from single cell transcriptomic data using previously trained machine learning models.

In some embodiments, the protein markers can be used to assign marker cell classifications to each cell in the first plurality of cells. In some embodiments, assigning marker cell classifications comprise manual gating of the marker cell marker values. In some embodiments, CD45+ CD14− CD19− CD3+ CD8+ cells are gated as CD8+ T cells. In some embodiments, CD45+ CD14− CD19+ CD3− cells are gated as B cells. In some embodiments, manual gating may be done by an expert, e.g. immunologist. In some embodiments, manual gating will be performed by converting the protein marker values into the FCS file format. In some embodiments, the marker cell classifications correspond to cell type, cell subtype, or cell state. In some embodiments, the marker cell classifications correspond to immune cell types. In some embodiments, the marker cell classifications correspond to the flow cell classifications that are described herein. In some embodiments, the marker cell classifications may comprise the immune cell populations in Table 1.

In some embodiments, the protein marker data comprise ADT protein marker expression. In some embodiments, the protein markers comprise TotalSeq-C DNA-tagged antibodies corresponding to the cell surface receptor proteins in the flow cytometry panels described herein. In some embodiments, the proteins markers comprise antibody markers against CD45, CD19, CD3, CD4, CD8, CD14, CD16, CD56, HLA-DR, TCRgd, CD5, CD38, PD-1/CD279, TCR Va7.2, TCR Va24-Ja18, TCR Vd2, CCR2/CD194, CCR6/CD196, CCR7/CD197, CCR10, CXCR3/CD183, CXCR5/CD185, CD45RA, CD45RO, CD95, CD27, CD28, CD25, CD127, CD122/IL2RB, CD31, CD39, CD161/KLRB1, CD103/ITGAE, ICOS/CD278, CD57, KLRG1, TIGIT, TIM-3/CD366, LAG-3/CD223, CD10, IgD, IgG, CD27, CD24, CD40, CD43, CD138, TACI/CD267, CD62L, CD319, CD303/BDCA-2/CLEC4C, CD123, CD11c, CD1c, CD141, CD294/CRTH2, CD335/NKp46, CD337/NKp30, CD69, CD86, and PD-L1/CD274.

TABLE 1
Non limiting examples of immune cell subpopulations identified with ADT
Cell Populations

WBC
B-cell
B-cell/CD5− CD27−
Monocyte/CD56+
Monocyte/CD56−
NK-cell
DC T-cell
iNKT
Gamma delta T- cells (Total GD)
Vd1
Vd2
Vdx
Mucosal-associated invariant T cells (MAIT)
TEMRA
CD4 NAÏVE
T_HELPER
CD4 Effector Memory
Treg
And over about 2000+ additional sub-populations

In some embodiments, methods further comprise processing the single cell transcriptomic and/or protein marker data. In some embodiments, processing the data comprises filtering out low-quality cells, doublets and empty droplets. In some embodiments, processing the data comprises normalizing transcriptomic and/or protein marker values between individual cells. In some embodiments, the data is processed to correct errors due to sequencing, batch effects, or technical drop-outs. In some embodiments, immune receptor sequence libraries are processed to perform clonotype analysis. A clonotype analysis may be used to provide a clonal expansion annotation for each cell, for example, for T cells and/or B cells.

### 3. Obtaining Fluorescent Intensity Data for Each Cell in the Second Plurality of Cells

In some embodiments, training a machine learning model to predict single cell transcriptomic values or profiles comprises obtaining fluorescent intensity data for each cell in a second plurality of cells. In some embodiments, fluorescent intensity data is generated using a flow cytometer to process fluorescently labeled cells. In some embodiments, the fluorescent intensity data comprise fluorescence values for a plurality of surface marker proteins. In some embodiments, the plurality of surface marker proteins comprises all or a subset of the surface proteins measured with protein markers in the first plurality of cells. In some embodiments, the methods comprise determining flow cell classification for each cell in the second plurality of cells related to the protein marker data. In some embodiments, the flow cell classifications correspond to the marker cell classifications assigned to the first plurality of cells.

In some embodiments, the fluorescent intensity data is obtained using flow cytometry. In some embodiments, the fluorescent intensity data are processed into flow cell classifications. In some embodiments, the fluorescent intensity data is obtained using flow cytometry followed by machine learning models to analyze cells and classify them using a standardized set of immune system status antibody panels.

In some embodiments, the fluorescent intensity data is obtained using flow cytometry. In some embodiments, the fluorescent intensity data is generated using a flow cytometry to process fluorescently labeled cells from the second plurality of cells. In some embodiments, the flow cytometer is configured for at least about 5, at least about 10, at least about 15, at least about 20, at least about 30, at least about 40, at least about 50, at least about 60, at least about 70, at least about 80, at least about 90, or at least about 100 fluorescent detection channels. In some embodiments, the flow cytometer is configured for between about 5 and about 100, between about 10 and about 90, between about 20 and about 80, between about 30 and about 70, or between about 40 and about 60 fluorescent detection channels. In some embodiments, the flow cytometry is a full spectrum flow cytometer.

In some embodiments, flow cytometry is performed on the second plurality of cells. In some embodiments, the samples are received at the laboratory facility, the sample is prepared and analyzed with flow cytometry. In some embodiments, preparing the sample comprises performing one or more of a dilution step, a centrifugation step, a staining step (using one or more fluorescently-labeled antibody panels) and/or a wash step.

In some embodiments, the staining step comprises contacting cells within at least a first aliquot of the second plurality of cells with at least a first panel (i.e., an immunophenotyping panel or flow cytometry panel) of fluorescently-labeled antibodies directed to a set of specific cell surface antigens (e.g., cell surface proteins) that collectively enable discrimination between the cell types or cell subtypes of interest. In some embodiments, the staining step comprises cells within at least a first aliquot of the second plurality of cells with at least a first panel (i.e., an immunophenotyping panel or flow cytometry panel) of fluorescently-labeled antibodies directed to a set of specific cell surface antigens (e.g., cell surface proteins) that collectively enable discrimination between the cell types or cell subtypes of interest. Sample processing may also include immunophenotyping panel design. A sample processing platform may comprise contacting each of one or more sample aliquots (e.g., 1, 2, 3, 4, 5, 6, 7, 8, 9, 10, or more than 10 sample aliquots) with one or more flow cytometry panels (e.g., 1, 2, 3, 4, 5, 6, 7, 8, 9, 10, or more than 10 flow cytometry panels).

In some embodiments, a flow cytometry panel or immunophenotyping panel may comprise at least about 5, at least about 10, at least about 15, at least about 20, at least about 30, at least about 40, at least about 50, at least about 60, at least about 70, at least about 80, at least about 90 or at least about 100 fluorescently-labeled antibodies directed to a set of cell surface antigens. In some embodiments, a flow cytometry panel or immunophenotyping panel may comprise between about 5 and about 100, between about 10 and about 90, between about 20 and about 80, between about 30 and about 70, or between about 40 and about 60 fluorescently-labeled antibodies directed to a set of cell surface antigens.

In some embodiments, each of two sample aliquots of the second plurality of cells may be stained with a different flow cytometry panel, one focusing on the antigen-presenting cell (APC) arm of the immune system (A panel), which comprises antibodies directed to a plurality different cell surface proteins, e.g. 36, and the other focusing on the adaptive arm of the immune systems (T panel), which comprises antibodies directed two a second plurality of cell surface markers, e.g. 41 cell surface proteins. In some instances, the panels may also include cell viability staining to distinguish between live cells and dead cells. In some instances, the panels may also comprise an autofluorescence measurement as a “marker”. Non-limiting examples of the cell surface proteins and additional markers that may be included in these panels are listed in Table 2.

TABLE 2
Non-limiting examples of cell surface receptor proteins and other
markers for distinguishing between immune cell sub-populations.

| APC panel markers (A panel) | T panel markers (T panel) |
|---|---|
| IGM, LIVE_DEAD_BLUE, CD5, CD62L, | TIGIT, CD5, CD28, CXCR5, CD39, TIM3, |
| CD294, CD69, CD38, PD1, CD11C, CD3, | CD38, PD1, TCRVA7_2_TCRVD1, CD95, |
| CD8, HLADR, CD24, CD337, CD123, | CD3, CD8, HLADR, CD31, CCR4, CCR6, |
| CD141, Autofluorescence 1, CD1C, CD4, | CCR7, Autofluorescence 1, CD57, ICOS, |
| TACI, CD319, CD335, PDL1, CD10, CD45, | CD4, KLRG1, TCRVA24_JA18, CD122, |
| CD16, IGD, CD40, CD19_TCRGD, CD43, | CD103, CXCR3, TCRVD2, CD45, CCR10, |
| CD14, CD138, CD15, CD56, CD86, CD303, | CD16, CD25, CD161, CD19_TCRGD, |
| CD27 | LAG3, CD14, CD45RO, CD56, CD127, |
| | CD45RA, CD27 |

In some embodiments, an additional sample aliquot of the second plurality of cell may be stained with a panel focusing on T intracellular proteins (T intracellular panel, TIC). In some embodiments, the TIC panel may comprise markers for CD45, CD14, CD19, CD3, CD4, CD8, TCRgd, CD56, CD16, CCR2, CCR4, CCR6, CCR7, CXCR3, CXCR4, CXCR5, CX3CR1, CD45RA, CD27, KI67, Granzyme B, FOXP3, TBET, GATA3, EOMES, BLIMPI, CTLA4, TCF1, TOX, BATF, IRF4, LEF1, and ZEB2.

In some embodiments, the panels include markers for determining immune cell type, immune system activation, lineage (e.g., the main marker(s) that are commonly used to define a certain cell population prior to further subsetting the cell type; examples include, but are not limited to, CD3 to define total T cells, and CD56 and CD16 to define natural killer cells), and exhaustion (cells that express markers associated with “cell exhaustion” (e.g., PD-1, TIGIT) can no longer proliferate and lose their functionalities as a result of chronic stimulation/prolonged activation of immune response).

In some embodiments, the fluorescent intensity data is obtained using flow cytometry and is in the form of a flow cytometry standard FCS file. In some embodiments, the FCS file may comprise, for example, fluorescence intensity data for one or more fluorescence detection channels (e.g., 2, 3, 4, 5, 6, 7, 8, 9, 10, 11, 12, 13, 14, 15, 20, 25, 30, 35, 40, 50, or more than 50 fluorescence detection channels, as well as data derived therefrom (e.g., forward scatter height data, forward scatter area data, side scatter height data, side scatter area data, autofluorescence data, or any combination thereof). In some instances, the number of fluorescence detection channels available may be determined by, for example, a combination of the detection hardware available as part of the flow cytometry instrument (e.g., comprising 5, 10, 20, 25, 50, 75, 100, 125, 150, 175, 200, or more than 200 detectors) and the number of spectrally-distinct fluorophores (e.g., 5, 10, 20, 25, 30, 35, 40, 45, 50, 60, or more than 60 spectrally-distinct fluorophores).

In some embodiments, manual gating may be used to determine flow cell classification for the second plurality of cells. In some embodiments, manual gating results in about 10-2500, about 200-2500, about 1000-2500 cell classification, or 2000-2300 cell classification. In some embodiments, manual gating may be performed by an expert, e.g. an immunologist. In some embodiments, the cell classification may relate to cell types, cell subtypes, or cell states.

In some embodiments, trained machine learning models may be used to determine flow cell classifications for the second plurality of cells. In some embodiments, the models trained using the method described in U.S. Ser. No. 18/353,022. In some embodiments, trained machine learning models are used to produce predictions of cell type or subtype (e.g., immune cell sub-population) for individual cell detection events and to determine cell counts (or frequencies) for each of a plurality of distinct cell types or subtypes. In some embodiments, the trained machine learning model uses a common hierarchy (a.k.a, a gating tree) to process the fluorescence profile data for each detected event and determine which and how many events belong to each measured populations (e.g., immune cell sub-population) in the hierarchy. In some embodiments, this may comprise over about 200 gates for the APC panel and over about 2000 gates for the T cell panel. The advantages of using this approach can be found in U.S. Ser. No. 18/353,022 incorporated by reference in its entirety.

In some embodiments, the flow cell characterizations may comprise the immune cell populations outlined in Table 1.

### 4. Generating Pseudo-Fluorescent Cell Classifications and Pseudo-Fluorescent Marker Data

In some embodiments, training a machine learning model to predict single cell transcriptomic values and/or profiles comprise generating pseudo-fluorescent data for the first plurality of cells. In some embodiments, the pseudo-fluorescent data is pseudo-fluorescent cell classifications. In some embodiments, the pseudo-fluorescent data is pseudo-fluorescent cell marker data. In some embodiments, the pseudo-fluorescent data is pseudo-fluorescent cell classifications and pseudo-fluorescent cell marker data.

Generating pseudo-fluorescent data solves the problem that the gene expression data and flow cytometry data is collected on two different pluralities of cells. Generating pseudo-fluorescent data for the first plurality of cells matches the population, subpopulation, or protein marker level data between the protein marker-derived data and the flow cytometry data. In some embodiments, the pseudo fluorescent data allows the machine learning model to recognize data derived from the first plurality of cells as data generated by flow cytometry.

In some embodiments, training a machine learning model to predict single cell transcriptomic values and/or profiles comprises generating pseudo-fluorescent cell classifications. Pseudo-fluorescent cell classifications may be cell classifications for cells in the first plurality of cells. Generating pseudo-fluorescent cell classification may comprise matching the marker cell classifications for each cell in the first plurality of cells to the flow cell classification for each cell classifications in the second plurality of cells. In some embodiments, the method comprises assigning a flow cell classification that is biologically similar to the marker cell classification to the cells that had previously been assigned marker cell classifications. In some embodiments, the methods may comprise parallel gating of the protein marker data for the first plurality of cells and the fluorescence intensity data for the second plurality of cells. In some embodiments, the method comprises aligning the marker cell classifications and the flow cell classifications. In some embodiments, protein marker data will be measured directly, simultaneously with the single cell transcriptomic data. In some embodiments, protein marker data or pseudo-fluorescence data derived thereof will be predicted by trained machine learning models from the single cell gene expression data. In some embodiments, the pseudo-fluorescent cell classifications may represent a cell type, cell subtype, cell population, or cell subpopulation.

In some embodiments, training a machine learning model to predict single cell transcriptomic values comprises generating or predicting pseudo-fluorescent cell marker data. There is a technical challenge to align the data generated by protein markers and data generated by flow cytometry. These data types are both on a continuous scale but the signals and representations of cells lacking biological expression of each protein differ. Training a machine learning model on the unaligned data would decrease the ability of the model to recognize the connection between single cell gene expression and flow cytometry data once it is employed. Aligning these data by creating pseudo-fluorescent cell marker data solves the problem.

In some embodiments, generating pseudo-fluorescent cell marker data comprises a linear transformation of the protein marker data. In some embodiments, the linear transformation can be done on a per channel basis by matching the proteins represented in the protein marker data from the first plurality of cells to the proteins represented in the fluorescence intensity data from the second plurality of cells. In some embodiments, applying a linear transformation to protein marker data from the first plurality of cells transforms the distributions to the same natural coordinate space as the fluorescent intensity data.

In some embodiments, the linear transformation may comprise any linear transformation method known in the art, e.g. learning a matrix transformation or correlation alignment. In some embodiments, the linear transformation can be done on a high dimensional basis by matching the totality, or subset, of the protein marker data represented in the protein marker data from the first plurality of cells to the totality, or matching subset, of the protein marker data represented in the fluorescence intensity data from the second plurality of cells. In some embodiments, applying a linear transformation to protein marker data from the first plurality of cells transforms the distributions to the same natural coordinate space as the fluorescent intensity data.

In some embodiments, generating pseudo-fluorescent cell markers data comprises a non-linear transformation of the protein marker data. In some embodiments, the non-linear transformation may use horizontal dataset integration algorithms. In some embodiments, the horizontal dataset integration algorithm may be Harmony (Korsunsky et al., Nature Methods, 16, 128901296 (2019)). In some embodiments, the non-linear transformation may comprise a machine learning harmonization of the protein's markers data into fluorescence intensity data.

In some embodiments, transforming the protein marker data comprises a harmonization approach The harmonization approach may take in matched fluorescent data (from flow cytometry) and protein marker data (ADT marker) And output single cell pseudo fluorescent data in the same format as the flow cytometry data. In some embodiments, harmonization approaches rely on batch correction methods known in the art. In some embodiments, the harmonization approach uses an empirical Bayes-based batch correction (e.g. ComBat) of self-organized map-defined cell clusters to the reference flow cytometry data. In some embodiments, the harmonization approach uses a nearest neighbor clustering of cells profiled with flow cytometry to cells profiled with protein marker data (ADT markers) using standard distance e.g. cosine similarity following batch correction of protein marker data into the flow cytometry latent space.

Occasionally, multiple mutually exclusive markers may be combined into the same fluorescence channel in glow cytometry (e.g. FSFC) and the markers will exist as separate channels in the parallel protein marker layer. In some embodiments, harmonization methods comprise Additively combining separate protein marker channels to produce hybrid channels ahead of harmonization.

In some embodiments, the non-linear transformation may be a machine learning technique. In some embodiments, the non-linear transformation may be a machine learning model trained to transpose high dimensional protein marker data into another high dimensional coordinate system. In some embodiments, the machine learning technique may use generative adversarial networks (GAN) to learn the non-linear transformation required, e.g. domain adaptation technique.

In some embodiments, transforming the protein marker data comprises providing the protein marker data for at least a subset of the first plurality of cells and the fluorescent intensity data for at least a subset of the second plurality of cells to a machine learning model configured to transform the protein marker data for the subset of the first plurality of cells into pseudo-fluorescent markers data related to the fluorescent intensity data for the subset of the second plurality of cells and output the pseudo-fluorescent markers data for the subset of the cells in the first plurality of cells.

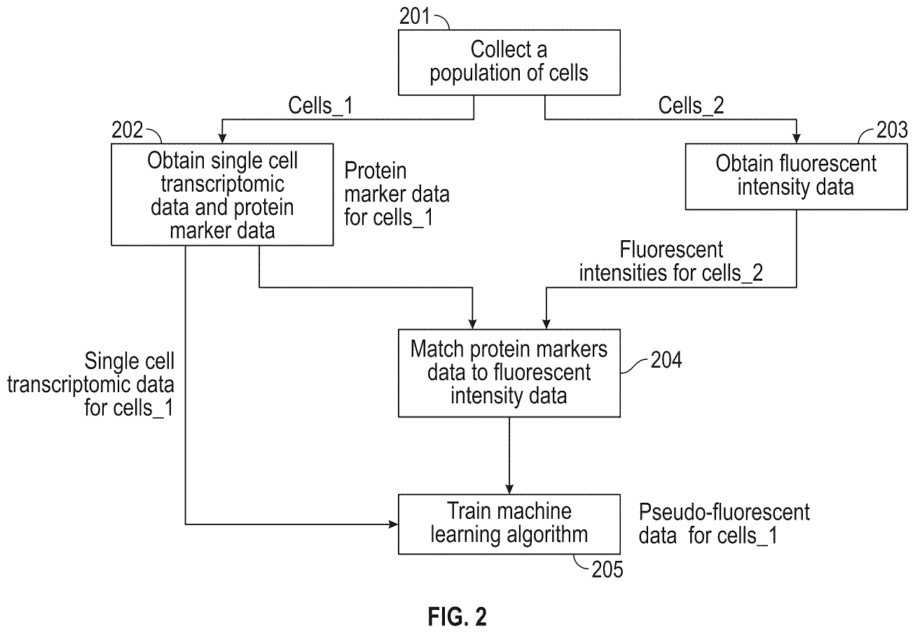

FIG. 2. provides a non-limiting example of training a machine learning model as described herein. The method comprises collecting a population of cells, 201, such as any of the cells described herein. A first plurality of cells, Cells_1, are inputs for obtaining single cell transcriptomic data and protein marker data, 202. In some embodiments, the transcriptomic data comprises single cell transcriptomic quantifications and/or clonal expansion statuses generated according to the methods described herein. In some embodiments, the protein marker data comprise ADT marker data as described herein. A second plurality of cells from the population of cells, cells_2 are inputs for obtaining fluorescent intensity data, 203. The protein marker data for cells_1 and the fluorescent intensity data for cells_2 are inputs for matching the protein marker data to the fluorescent intensity data, 204, to output pseudo-fluorescent data for cells_1. In some embodiments, the fluorescent intensity data may be data generated from the fluorescent intensity data such as cell classifications. In some embodiments, the pseudo-fluorescent data comprises pseudo-fluorescent protein marker data and/or pseudo-fluorescent cell classifications generated according to the methods described herein. The output single cell transcriptomic data for cells_1 and the pseudo-fluorescent data for cells_1 are inputs for training the machine learning model, 205. The machine learning model may be any of the machine learning models described herein. The machine learning model, 205, may be trained to output predicted single cell transcriptomic value, such as a predicted single cell transcriptomic quantification for a gene and/or a predicted clonal expansion status for a cell.

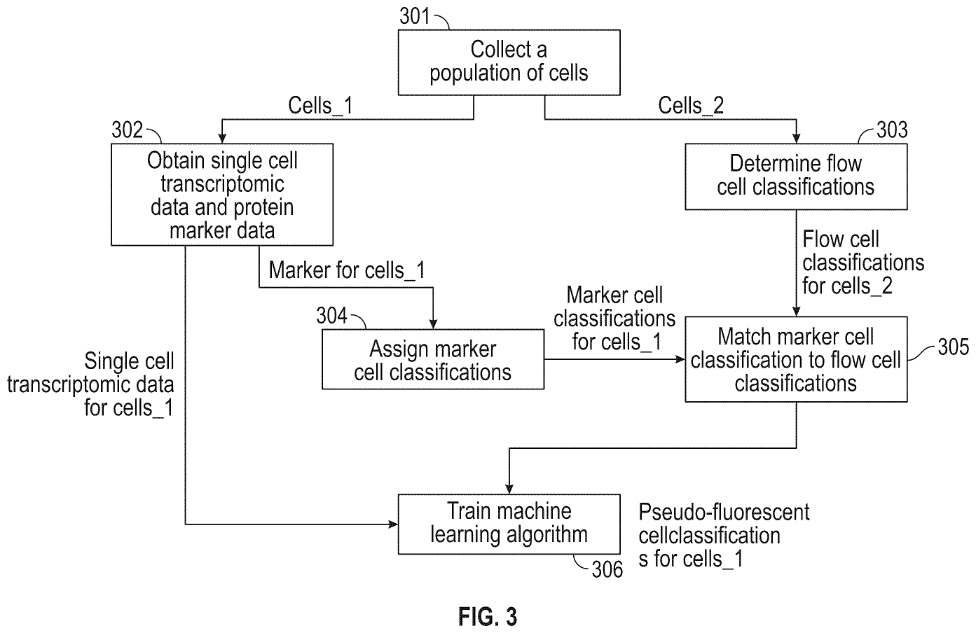

FIG. 3. provides a non-limiting example of training a machine learning model as described herein. The method comprises collecting a population of cells, 301. A first plurality of cells, Cells_1, are inputs for obtaining single cell transcriptomic data and protein marker data, 302. The marker data for cells_1 are inputs for assigning marker cell classification for cells_1, 304. A second plurality of cells from the population of cells, cells_2, are inputs for determining flow cell classifications, 303. The marker cell classification from 304 and the flow cell classifications from 303 are used as input for matching marker cell classifications to flow cell classifications, 305. The pseudo-fluorescent cell classifications from 305 and the single cell transcriptomic data from 302 are inputs for training a machine learning model, 306.

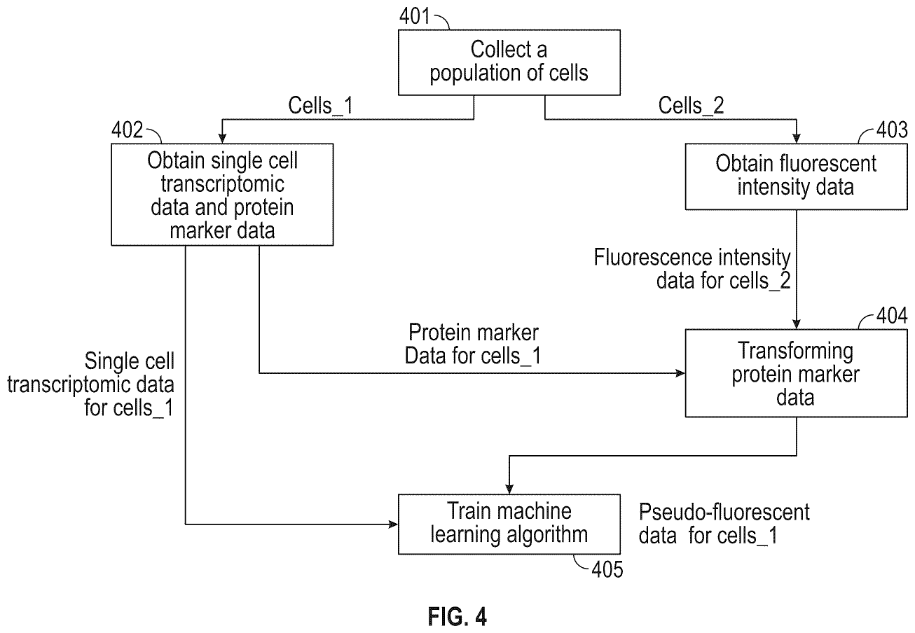

FIG. 4. provides a non-limiting example of training a machine learning model as described herein. The method comprises collecting a population of cells, 401. A first plurality of cells, Cells_1, are inputs for obtaining single cell transcriptomic data and protein marker data, 402. A second plurality of cells from the population of cells, cells_2 are inputs for obtaining fluorescent intensity data, 403. The protein marker data for cells_1 and the fluorescent intensity data for cells_2 are inputs for transforming the protein marker data into pseudo-fluorescent marker data for cells_1, 404. The output single cell transcriptomic data for cells_1 and the pseudo-fluorescent marker data for cells_1 are inputs for training the machine learning model, 405.

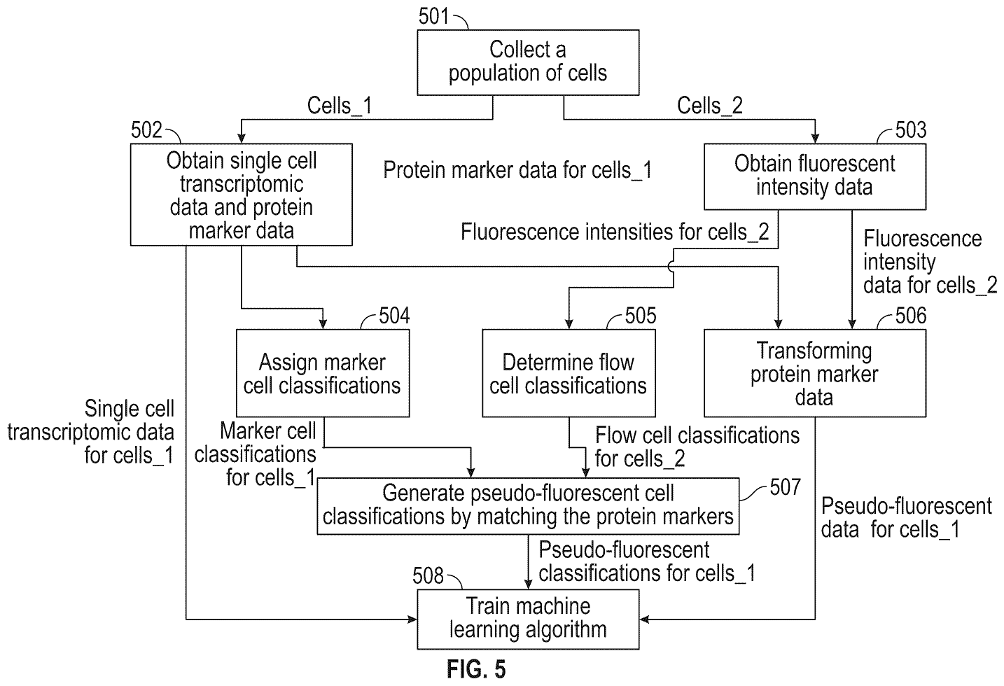

FIG. 5. provides a non-limiting example of training a machine learning model as described herein. The method comprises collecting a population of cells, 501. A first plurality of cells, Cells_1, are inputs for obtaining single cell transcriptomic data and protein marker data, 502. The protein marker data are input for assigning marker cell classification, 504, for cells_1. A second plurality of cells from the population of cells, cells_2, are inputs for determining flow cell classifications, 503. The fluorescence intensities for cells_2 are used as inputs for determining flow cell classifications, 505. The protein markers for cells_1 and the fluorescence intensity data for cells_2 are used as inputs for transforming the protein marker data, 506, into pseudo-fluorescent marker data for cells_1. The marker cell classifications for cells_1 and the flow cell classifications for cells_2 are used as inputs for generating pseudo-fluorescent cells classifications by matching protein markers, 507. The resulting pseudo-fluorescent classifications for cells_1, the pseudo-fluorescent marker data for cells_1 and the single cells transcriptomic data for cells_1 are used as inputs for training a machine learning model, 508.

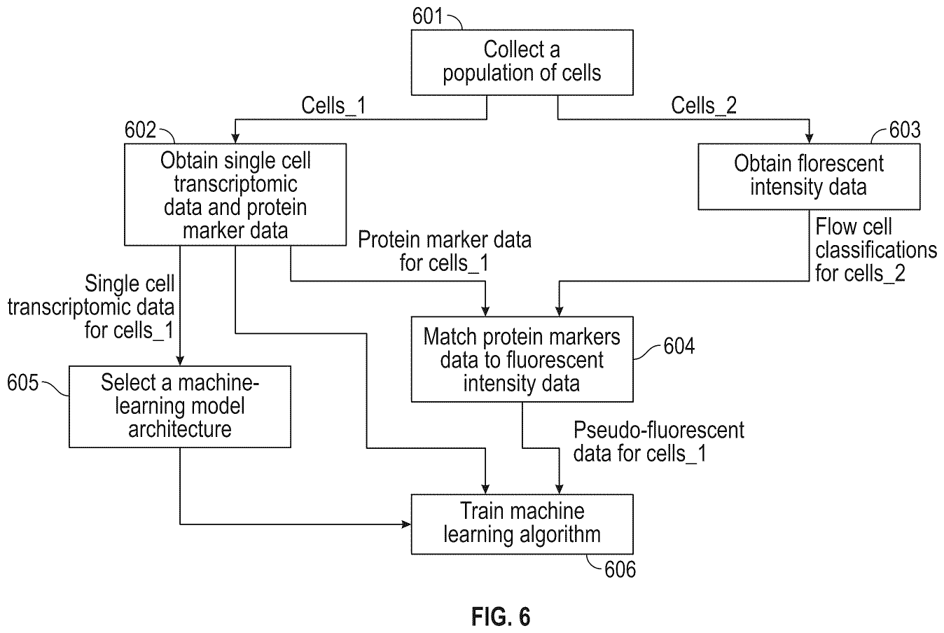

FIG. 6. provides a non-limiting example of training a machine learning model (e.g. machine learning model) as described herein. The machine learning model in FIG. 6 is a flexible machine learning model wherein the machine learning model architecture is chosen based on single cell transcriptomic data as described herein. The method comprises collecting a population of cells, 601, such as any of the cells described herein. A first plurality of cells, Cells_1, are inputs for obtaining single cell transcriptomic data and protein marker data, 602. In some embodiments, the transcriptomic data comprises single cell transcriptomic quantifications and/or clonal expansion statuses generated according to the methods described herein. In some embodiments, the protein marker data comprise ADT marker data as described herein. A second plurality of cells from the population of cells, cells_2 are inputs for obtaining fluorescent intensity data, 603. The protein marker data for cells_1 and the fluorescent intensity data for cells_2 are inputs for matching the protein marker data to the fluorescent intensity data, 604, to output pseudo-fluorescent data for cells_1. In some embodiments, the fluorescent intensity data may be data generated from the fluorescent intensity data such as cell classifications. In some embodiments, the pseudo-fluorescent data comprises pseudo-fluorescent protein marker data and/or pseudo-fluorescent cell classifications generated according to the methods described herein. The single cell transcriptomic data for cells_1 may be used to select a machine learning model architecture, 605. The machine learning model architecture may be selected based on the distribution of the single cell transcriptomic data (e.g. single cell transcriptomic quantifications). The machine learning model architecture may be a regression model, a hybrid model, or a classifier, as described herein. The output single cell transcriptomic data for cells_1, the pseudo-fluorescent data for cells_1, and the selected machine learning model architecture are inputs for training the machine learning model, 606. The machine learning model may be any of the machine learning models described herein. The machine learning model, 606, may be trained to output predicted single cell transcriptomic value, such as a predicted single cell transcriptomic quantification for a gene and/or a predicted clonal expansion status for a cell.

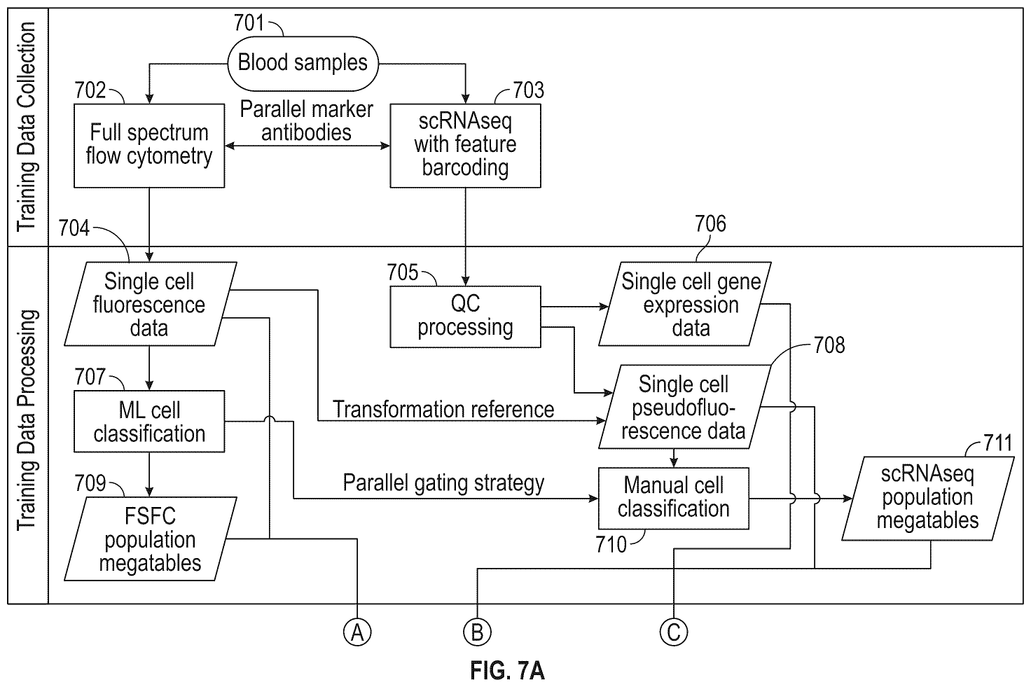

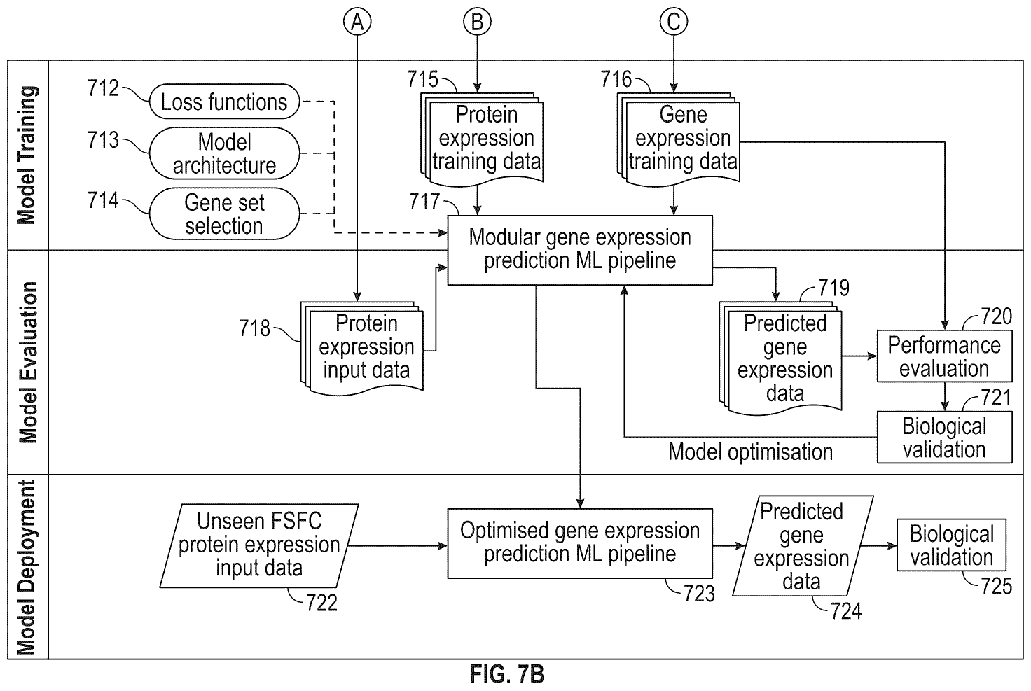

FIG. 7A-7B shows a non-limiting schematic of a method according to some of the methods and systems described herein. As shown in the non-limiting schematic the methods and systems may be arranged in a modular format to improve efficiency, reproducibility, and accuracy. The individual steps described in FIG. 7A and FIG. 7B present concepts described elsewhere in the application.

A data collection module may be responsible for collecting flow cytometry and single cell transcriptomic data from samples with parallel protein markers. (FIG. 7A) A training data processing module may be responsible for preparing the flow cytometry and single cell transcriptomic data for training the machine learning models described herein. In some embodiments, the training data processing module may generate the pseudo-fluorescence data as described herein. (FIG. 7A) At 701, blood samples may be used as input to 702 and 703. At 702, an aliquot of the blood sample from 701 may be used for full spectrum flow cytometry, and at 703 an aliquot of the blood samples may be used for scRNAseq with feature barcoding (e.g. CITE-seq). The markers for 702 and 7032 may be parallel marker antibodies for generation of pseudo-fluorescence data as described herein. The full spectrum flow cytometry data from 702 may be used to generate single cell fluorence data at 704, cell classifications may then be generated using a machine learning based cell classification method at 707. The single cell fluorescence data from 704 and the machine learning cell classifications may be used to generate full spectrum flow cytometry (FSFC) population megatables, 709. The FSFC population megatables at 709 may comprise characterization of the cellular composition of the sample used for the full spectrum flow cytometry at 702. Also in the training data processing module, the scRNAseq with feature barcoding from block 703 may go through quality control (QC) processes at 705 to output single cell gene expression data at 706 and single cell pseudo-fluorescence data, 708, using the transformation of the single cell fluorescence data from 704. The single cell pseudo-fluorescence data at 708 and the cell classifications from 707 may be used to classify the cells at 710. The cell classifications from 710 can be used to generate scRNAseq population megatables at block 711 which comprise cellular composition information about the aliquot of the sample used for scRNAseq with feature barcoding at 703.

A model training module may be used to load training data, evaluate the distribution of the training data, select a model architecture, and select an appropriate loss function. (FIG. 7B) The module may build the machine learning model and loss functions based on the pipeline parameters then configure and deploy the necessary infrastructure to run the process. Protein expression training data at block 715 may comprise single cell pseudo-fluorescence data from 708. Gene expression training data at block 716 may comprise single cell gene expression data from 706. The training data at blocks 715 and 716 may be input into a modular gene expression prediction machine learning pipeline at 717. The machine learning pipeline at 717 may be any of the flexible machine learning models as described herein. The machine learning pipeline at 717 may also take in loss function form 713, various model architecture, 713, and a gene set selection set (e.g. highly expressed genes), 714.

A model evaluation module may load validation data that can be used to test the machine learning model trained in the training module. The model evaluation module may configure and deploy the necessary infrastructure for predicting single cell transcriptomic data from the flow values in the validation data. The model evaluation module may load the paired evaluation data from the validation data and the prediction data then calculate the evaluation metrics based on the performance of the predicted versus evaluation data. (FIG. 7B) Protein expression input data, 718, such as cell classifications generated from flow cytometry data or single cell fluorescence data from flow cytometry may be used as evaluation data for model 717. Machine learning model pipeline 717 may be used to predict gene expression data, 719. The predicted gene expression data at 719, and the gene expression training data from 716, may be used for evaluating performance of the model at 720. The performance evaluation may be used for biological validation t 721. The biological validation at 721, may be used for optimization of model 717. The process may be repeated to further optimize the model.

A model deployment module can be used for generating a single cell transcriptomic profile for a subject using flow cytometry data that the machine learning model has not yet seen. (FIG. 7B). At 722, full spectrum flow cytometry (FSFC) protein expression input data that has not previously been seen by the machine learning model at 717 may be used as input to the optimized gene expression prediction machine learning pipeline at 723. The optimized gene expression prediction machine learning pipeline may be used to generate predicted gene expression data, 724. At 725, the predicted gene expression data may be biologically validated using methods described herein,

### D. Optional Validation Methods

In someone embodiments, the methods described herein include methods for validating the predictions. In some embodiments, the validations comprised biological validations of the predicted clonal expansion statuses and/or predictable predicted single cell transcriptomic quantifications. In some embodiments, validation may be used to accommodate for the substantial complexity in the predictions challenged by the lack of cell to cell registration across the ground truth transcriptomic data used for training and the predicted single cell transcriptomic values. The ground truth transcriptomic data may be transcriptomic measured using single cell RNA sequencing methods known in the art and described herein. In some embodiments, biological validation may comprise sample level evaluation of biologically known phenomena.

In some embodiments, validation may comprise assessment of global data structure Through dimensionality reduction and clustering Common approaches may be used to interpret population structure in the data and to compare this to known biological phenomena. The validation methods may comprise visual inspection of UMP embeddings or quantitative analysis of cluster identity using silhouette scores can be used to assess accuracy of the cell clustering global transcriptome. Once cluster embodiments have been assigned from predicted matrices, assessment of cell frequencies against ground truth matrices and matched, ground truth flow cytometry samples can quantify the accuracy of global immune structure recapitulation from the predictions. In some embodiments, the directionality of population frequency changes against metadata features about the sample can be used for validation For example, a decrease in naive CD8 T cells with age is well characterized, which can be assessed in the predicted cell frequencies. Concordance of global gene expression structure can be assessed by combining actual and predicted gene expression matrices, and performing dimensionality reduction on this combined dataset. Using the same analytical approaches as described herein, consistency in UMAP embeddings across actual and predicted batches can be assessed. Batch correction across ground truth and predicted values may be performed to understand if any discordance between the two can be adjusted for

In some embodiments, validation may comprise assessing gene level signatures. Methods for assessing gene level signatures are known in the art. In some embodiments, differential expression testing may be used to identify cluster-defining markers, which will be compared to canonical population markers from the literature. For example, CD14 should be highly and selectively expressed in classical monocytes. Cell type recognition tools with reference databases containing gene-level information on pure immune populations can be used. Correlation matrix analysis can be performed to identify concordance in co-correlating modules of gene expression across ground truth and predicted gene expression values.

In some embodiments, validation may comprise assessing pathway level signatures. For example, pathway annotation in published repositories may be used to understand if patterns in functional pathways are consistent across ground truth and predicted values. Tools in the art can be used for assessing pathway activity in single cell transcriptomic data, for example but not limited to SCPA, AUCell, fGSEA, GSVA, VISION, and iDEA. In some embodiments, a one-vs-all comparison of pathway signatures can be used to assess the consistency across ground truth vs predicted clusters. For example, comparing pathways in naive CD4 T cells to all other clusters and assessing the consistency of p-values from all comparisons in actual vs predicted groups.

### III. Systems for Determining Single Cell Transcriptomic Values and Determining a Single Cells Transcriptomic Profile for a Subject from Flow Cytometry

Also disclosed herein are systems designed to implement any of the disclosed methods for determining a predicted single cell transcriptomic value (e.g. Single cell transcriptomic quantifications and/or clonal expansion statuses) or generating a single cell transcriptomic profile for a subject. The systems may comprise e.g., one or more processors, and a memory unit communicatively coupled to the one or more processors and configured to store instructions that, when executed by the one or more processors, cause the system to: contact at least a first aliquot of a sample from a subject with at least a first immunophenotyping panel to fluorescently label cells contained within the sample; process the fluorescently-labeled cell using a flow cytometer to generate fluorescent intensity data, or data derived therefrom, for a plurality of fluorescently-labeled cells from the sample; provide only at least a subset of the fluorescent intensity data, or data derived therefrom for the plurality of fluorescently-labeled cells as input to a machine learning model trained using single cell transcriptomics data for a first plurality of cells and pseudo-fluorescent data for the first plurality of cells; generate a predicted single cell transcriptomic value for the fluorescently-labeled cells using the trained machine learning model for a plurality of genes, thereby generating a single cell transcriptomic profile for the subject. In some instances, the system may further comprise a flow cytometer instrument.

In some instances, the systems may comprise e.g., one or more processors, and a memory unit communicatively coupled to the one or more processors and configured to store instructions that, when executed by the one or more processors, cause the system to perform any of the embodiments disclosed herein.

Similarly, non-transitory computer-readable storage media are disclosed that may comprise instructions for operating a system configured to perform any of the disclosed methods for determining a single cell transcriptomic value or generating a single cell transcriptomic profile for a subject. For example, non-transitory computer-readable storage media storing one or more programs are described, the one or more programs comprising instructions, which when executed by one or more processors of a system, cause the system to: contact at least a first aliquot of a sample from a subject with at least a first immunophenotyping panel to fluorescently label cells contained within the sample; process the fluorescently-labeled cell using a flow cytometer to generate fluorescent intensity data, or data derived therefrom, for a plurality of fluorescently-labeled cells from the sample; provide only at least a subset of the fluorescent intensity data, or data derived therefrom for the plurality of fluorescently-labeled cells as input to a machine learning model trained using single cell transcriptomics data for a first plurality of cells and pseudo-fluorescent data for the first plurality of cells; generate a predicted single cell transcriptomic value for the fluorescently-labeled cells using the trained machine learning model for a plurality of genes, thereby generating a single cell transcriptomic profile for the subject. In some instances, the system may further comprise a flow cytometer instrument.

In some embodiments, the non-transitory computer-readable storage media storing one or more programs are described, the one or more programs comprising instructions, which when executed by one or more processors of a system, cause the system to perform any of the embodiments disclosed herein.

### A. Computer Processors & Computing Systems

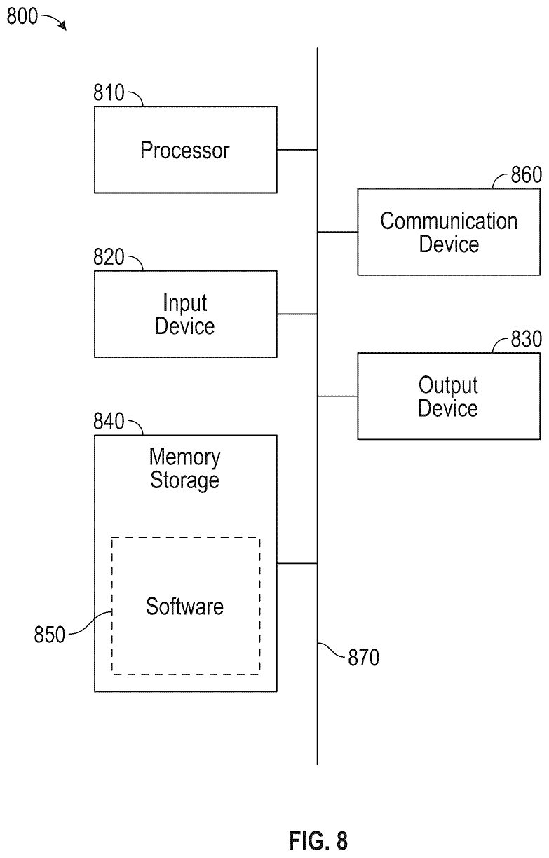

FIG. 8 illustrates an exemplary computing system, in accordance with some implementations. Computing system 800 can be a component of a system for determining a single cell expression profile for a subject.

Computing system 800 can include a host computer connected to a network. Computing system 800 can be a client computer or a server. As shown in FIG. 8, computing system 800 can comprise any suitable type of microprocessor-based device, such as a personal computer; workstation; server; or handheld computing device, such as a phone or tablet. The computer can include, for example, one or more of processor 810, input device 820, output device 830, memory storage 840, and communication device 860.

Input device 820 can be any suitable device that provides input, such as a touch screen or monitor, keyboard, mouse, or voice-recognition device. Using the Input device, a user may select a machine learning architecture for the machine learning model based on a independent or external review of the distribution of the training data. Output device 830 can be any suitable device that provides output, such as a touch screen, monitor, printer, disk drive, or speaker.

Memory storage 840 can be any suitable device that provides storage, such as an electrical, magnetic, or optical memory, including a RAM, cache, hard drive, CD-ROM drive, tape drive, or removable storage disk. Communication device 860 can include any suitable device capable of transmitting and receiving signals over a network, such as a network interface chip or card. The components of the computer can be connected in any suitable manner, such as via a physical bus or wirelessly. Memory storage 840 can be a non-transitory computer-readable storage medium comprising one or more programs, which, when executed by one or more processors, such as processor 810, cause the one or more processors to execute any of the methods described herein.

Software 850, which can be stored in memory storage 740 and executed by processor 810, can include, for example, the programming that embodies the functionality of the present disclosure (e.g., as embodied in the methods, systems, computers, servers, and/or devices as described above). In some embodiments, software 850 can be implemented and executed on a combination of servers such as application servers and database servers.

Software 850 can also be stored and/or transported within any computer-readable storage medium for use by or in connection with an instruction execution system, apparatus, or device, such as those described above, that can fetch and execute instructions associated with the software from the instruction execution system, apparatus, or device. In the context of this disclosure, a computer-readable storage medium can be any medium, such as storage 840, that can contain or store programming for use by or in connection with an instruction execution system, apparatus, or device.

Software 850 can also be propagated within any transport medium for use by or in connection with an instruction execution system, apparatus, or device, such as those described above, that can fetch and execute instructions associated with the software from the instruction execution system, apparatus, or device. In the context of this disclosure, a transport medium can be any medium that can communicate, propagate, or transport programming for use by or in connection with an instruction execution system, apparatus, or device. The transport-readable medium can include, but is not limited to, an electronic, magnetic, optical, electromagnetic, or infrared wired or wireless propagation medium.

Computing system 800 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computing system 800 can implement any operating system suitable for operating on the network. Software 850 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

### EXAMPLE EMBODIMENTS

The following embodiments are exemplary of the invention described herein and are not intended to limit the scope of the invention.
1. A method for determining a single cell transcriptomic value from flow cytometry data, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cells from the sample; providing only at least a subset of the fluorescent intensity data, or data derived therefrom for the plurality of fluorescently-labeled cells input to a machine learning model trained using single cell transcriptomics data for a first plurality of cells and pseudo-fluorescent data for the first plurality of cells; generating a single cell gene transcriptomic value for the fluorescently-labeled cells using the trained machine learning model.
2. A method for determining a single cell transcriptomic profile for a subject, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cell from the sample; providing only at least a subset of the fluorescent intensity data, or data derived therefrom for the plurality of fluorescently-labeled cells input to a machine learning model trained using single cell transcriptomics data for a first plurality of cells and pseudo-fluorescent data for the first plurality of cells; generating a single cell transcriptomic value for the fluorescently-labeled cells using the trained machine learning model for a plurality of genes, thereby generating a single cell transcriptomic profile for the subject.
3. The method of any of embodiments 1 or 2, wherein the machine learning model is organized as a plurality of nodes, wherein each node comprises an individual machine learning model.
4. The method of any of embodiments 1-3, wherein the pseudo-fluorescent data for the first plurality of cells is generated by matching at least a subset of protein marker data for each cell in the first plurality of cells to fluorescent intensity data for each cell in a second plurality of cells.
5. The method of any of embodiments 1-4, wherein the matching at least a subset of protein markers comprises transforming the proteins marker data.
6. The method of embodiment 5, wherein the transforming the protein marker data comprises a linear transformation.
7. The method of embodiment 5, wherein the transforming the protein marker data comprises a non-linear transformation.
8. The method of embodiment 5, wherein transforming the protein marker data comprises providing the protein marker data for at least a subset of the first plurality of cells and the fluorescent intensity data for at least a subset of the second plurality of cells to a machine learning model configured to transform the protein marker data for the subset of the first plurality of cells into pseudo-fluorescent marker data related to the fluorescent intensity data for the subset of the second plurality of cells and output the pseudo-fluorescent marker data for the subset of the cells in the first plurality of cells.
9. The method of any of embodiments 1-8, wherein the single cell transcriptomic data for the first plurality of cells is generated using a method for characterizing each cell in the first plurality of cells by simultaneous detection of a plurality of protein marker data and single cell transcriptomic values for a plurality of genes.
10. The method of any of embodiments 1-8, wherein the transcriptomic data for the first plurality of cells is generated by immune receptor profiling.
11. The method of any of embodiments 4-8, wherein the protein marker data is generated by providing at least a subset of the single cell transcriptomic data for the first plurality of cells to a machine learning model configured to transform the at least a subset of single cell transcriptomic data for the first plurality of cells into protein marker data and output protein marker data for the first plurality of cells.
12. The method of any of embodiments 4-11, wherein the fluorescent intensity data for each cell in the second plurality of cells is generated using a flow cytometer to process fluorescently-labeled cells from the second plurality of cells.
13. The method of embodiment 12, wherein the flow cytometer is configured for at least about 5, at least about 10, at least about 15, at least about 20, at least about 30, at least about 40, at least about 50, at least about 60, at least about 70, at least about 80, at least about 90, or at least about 100 fluorescence detection channels.
14. The method of embodiment 12, wherein the flow cytometer is a full spectrum flow cytometer.
15. The method of any of embodiment 1-14, wherein the pseudo-fluorescent data for the first plurality of cells is pseudo-fluorescent cell classifications data for at least a subset of the first plurality of cells.
16. The method of any of embodiments 1-14, wherein the pseudo-fluorescent data for the first plurality of cells further comprises pseudo-fluorescent cell classifications data for at least a subset of the first plurality of cells.
17. The method of embodiments 15 or 16, wherein the pseudo-fluorescent cell classification data are generated by assigning a pseudo-fluorescent cell classification to each cell related to the protein marker data that corresponds to each cell in the first plurality of cells.
18. The method of any of embodiments 1-17, wherein data derived from the fluorescent intensity data are flow cell classifications for each cell in the second plurality of cells.
19. The method of any of embodiments 1-18, wherein the machine model is trained to determine a single cell gene expression value.
20. The method of any of embodiments 2-19, wherein the single cell transcriptomic profile comprises a single cell transcriptomic value for at least about 100, about 1,000, about 5000, about 10000, or about 20,000 genes.
21. The method of any of embodiments 2-20, wherein the single cell transcriptomic profile comprises emergent properties of single cell transcriptomic data relevant to groups of cells in the sample.
22. The method of embodiment 21, wherein the emergent properties of single cell transcriptomic data relevant to groups of cells in the sample comprise gene signatures, cell trajectories and/or transcriptional patterns.
23. The method of any of embodiments 2-20, wherein the single cell transcriptomic profile comprises emergent properties of single cell immune receptor profiling for groups of cells in the sample.
24. The method of embodiment 23, wherein the emergent properties of single cell immune receptor profiling for groups of cells in the sample comprise immune receptor diversity and/or predictions of clonal expansions.
25. The method of any of embodiments 2-24, wherein the single cell transcriptomic profile is used to diagnose an immune-related disease or disorder, monitor progression of an immune-related disease or disorder, or monitor a response to treatment of an immune-related disease or disorder in the subject.
26. A method for training a machine learning algorithm to generate a predicted single cell transcriptomic value, comprising; collecting a population of cells comprising a first plurality of cells and a second plurality of cells; obtaining single cell transcriptomic data and protein marker data for each cell in the first plurality of cells, wherein the single cell transcriptomic data comprise a single cell transcriptomic value for a plurality of genes; obtaining fluorescent intensity data for each cell in the second plurality of cells generated using a flow cytometer to process fluorescently-labeled cells from the second plurality of cells; generating pseudo-fluorescent data by matching at least a subset of the protein marker data for each cell in the first plurality of cells to the fluorescent intensity data for each cell in the second plurality of cells; and training a machine-learning model to generate a predicted single cell transcriptomic value, wherein the training is based on the transcriptomic data for at least a subset of the first plurality of cells and the pseudo-fluorescent data for the at least a subset of the first plurality of cells.
27. The method of embodiment 26, wherein the single cell transcriptomic data and protein marker data for each cell in the first plurality of cells are generated using a method for characterizing a cell by simultaneous detection of a plurality of protein markers and single cell transcriptomic values for a plurality of genes.
28. The method of any of embodiment 26, wherein the single cell transcriptomic data for the first plurality of cells is generated by immune receptor profiling.
29. The method of embodiment 26, wherein the protein marker data is generated by providing at least a subset of the single cell transcriptomic data for the first plurality of cells to a machine learning model configured to transform the at least a subset of single cell transcriptomic data for the first plurality of cells into protein marker data and output protein marker data for the first plurality of cells.
30. A method for training a machine learning algorithm to generate a predicted single cell transcriptomic value, comprising; collecting a population of cells, comprising a first plurality of cells and a second plurality of cells; obtaining single cell transcriptomic data and protein marker data for each cell in the first plurality of cells, wherein the single cell transcriptomic data comprise a transcriptomic value for a first plurality of genes, wherein the protein marker data comprise a marker value for a plurality of surface proteins, assigning a marker cell classification for each cell related to the protein marker data that corresponds to each cell in the first plurality of cells; determining a flow cell classification for each cell in the second plurality of cells by processing data generated using a flow cytometer to process fluorescently-labeled cells from the second plurality of cells; generate pseudo-fluorescent cell classification data by matching the marker cell classifications for each cell in the first plurality of cells to the flow cell classifications for each cell in the second plurality of cells; and training a machine-learning model to generate a predicted single cell transcriptomic value, wherein the training is based on the transcriptomic data for at least a subset of the first plurality of cells and the pseudo-fluorescent cell classification data for the at least a subset of the first plurality of cells.
31. The method of embodiment 30, wherein the single cell transcriptomic data and protein marker data for each cell in the first plurality of cells are generated using a method for characterizing a cell by simultaneous detection of a plurality of protein markers and transcriptomic values for a plurality of genes.
32. The method of embodiment 31, wherein the protein marker data comprise an antibody-derived tag (ADT) marker value for a plurality of surface proteins.
33. The method of embodiment 30, wherein the single cell transcriptomic data for the first plurality of cells is generated by immune receptor profiling.
34. The method of embodiment 30, wherein the protein marker data is generated by providing at least a subset of the single cell transcriptomic data for the first plurality of cells to a machine learning model configured to transform the at least a subset of single cell transcriptomic data for the first plurality of cells into protein marker data and output protein marker data for the first plurality of cells.
35. A method for training a machine learning algorithm to generate a predicted single cell transcriptomic value for a plurality of genes, comprising; collecting a population of cells comprising a first plurality of cells and a second plurality of cells; obtaining single cell transcriptomic data and protein marker data for each cell in the first plurality of cells, wherein the single cell transcriptomic data comprise a transcriptomic value for a plurality of genes, wherein the protein marker data comprise a value for a plurality of surface marker proteins; obtaining fluorescent intensity data for each cell in the second plurality of cells using a flow cytometer to process fluorescently-labeled cells from the second plurality of cells, wherein the fluorescent intensity data comprise fluorescence values for the plurality of surface marker proteins; generating pseudo-fluorescent markers for the first plurality of cells by transforming the at least a subset of the protein marker data for the first plurality of cells into pseudo-fluorescent markers data related to the fluorescent intensity data for the subset of the second plurality of cells; and training a machine-learning model to generate a predicted single cell transcriptomic value, wherein the training is based on the single cell transcriptomic data for at least a subset of the first plurality of cells and the pseudo-fluorescent cell markers data for the at least a subset of the first plurality of cells.
36. The method of embodiment 35, wherein the single cell transcriptomic data and protein marker data for each cell in the first plurality of cells are generated using a method for characterizing a cell by simultaneous detection of a plurality of protein markers and transcriptomic values for a plurality of genes.
37. The method of embodiment 36, wherein the protein marker data comprise an ADT marker value for a plurality of surface proteins.
38. The method of embodiment 35, wherein the single cell transcriptomic data for the first plurality of cells is generated by immune receptor profiling.
39. The method of embodiment 35, wherein the protein marker data is generated by providing at least a subset of the single cell transcriptomic data for the first plurality of cells to a machine learning model configured to transform the at least a subset of single cell transcriptomic data for the first plurality of cells into protein marker data and output protein marker data for the first plurality of cells.
40. The method of any of embodiments 35-39, wherein the transforming the protein marker data comprises a linear transformation.
41. The method of any of embodiments 35-39, wherein the transforming the protein marker data comprises a non-linear transformation.
42. The method of any of embodiments 35-39, wherein the transforming the protein marker data comprises providing the protein marker data for at least a subset of the first plurality of cells and the fluorescent intensity data for at least a subset of the second plurality of cells to a machine learning model configured to transform the protein marker data for the subset of the first plurality of cells into pseudo-fluorescent marker data related to the fluorescent intensity data for the subset of the second plurality of cells and output the pseudo-fluorescent marker data for the subset of the cells in the first plurality of cells.
43. A method for training a machine learning algorithm to generate a predicted single cell transcriptomic value for a plurality of genes, comprising; collecting a population of cells comprising a first plurality of cells and a second plurality of cells; obtaining single cell transcriptomic data and protein marker data for each cell in the first plurality of cells, wherein the single cell transcriptomic data comprise a transcriptomic value for a plurality of genes, wherein the protein marker data comprise a marker value for a plurality of surface marker proteins; assigning a marker cell classification for each cell related to the protein marker data that corresponds to each cell in the first plurality of cells; obtaining fluorescent intensity data for each cell in the second plurality of cells using a flow cytometer to process fluorescently-labeled cells from the second plurality of cells, wherein the fluorescent intensity data a fluorescent intensity value for the plurality of surface marker proteins; determining a flow cell classification for each cell in the second plurality of cells by processing the flow intensity data; generating pseudo-fluorescent cell data by matching the marker cell classifications for each cell in the first plurality of cells to the flow cell classifications for each cell in the second plurality of cells; generating pseudo-fluorescent markers for the first plurality of cells by transforming at least a subset of the protein marker data for the first plurality of cells into pseudo-fluorescent markers data related to the fluorescent intensity data for the subset of the second plurality of cells; and training a machine-learning model to generate a predicted single cell gene expression value, wherein the training is based on the single cell transcriptomic data for at least a subset of the first plurality of cells, the pseudo-fluorescent cell markers data for the at least a subset of the first plurality of cells, and the pseudo-fluorescent cell classification data for the at least a subset of the first plurality of cells.
44. The method of embodiment 43, wherein the single cell transcriptomic data and protein marker data for each cell in the first plurality of cells are generated using a for characterizing a cell by simultaneous detection of a plurality of protein markers and transcriptomic values for a plurality of genes.
45. The method of embodiment 44, wherein the protein marker data comprise an ADT marker value for a plurality of surface proteins.
46. The method of embodiment 43, wherein the single cell transcriptomic data for the first plurality of cells is generated by immune receptor profiling.
47. The method of embodiment 43, wherein the protein marker data is generated by providing at least a subset of the single cell transcriptomic data for the first plurality of cells to a machine learning model configured to transform the at least a subset of single cell transcriptomic data for the first plurality of cells into protein marker data and output protein marker data for the first plurality of cells.
48. The method of any of embodiments 43-47, wherein the transforming the protein marker data comprises a linear transformation.
49. The method of any of embodiments 43-47, wherein the transforming the protein marker data comprises a non-linear transformation.
50. The method of any of embodiments 43-47, wherein the transforming the protein marker data comprises providing the protein marker data for at least a subset of the first plurality of cells and the fluorescent intensity data for at least a subset of the second plurality of cells to a machine learning model configured to transform the protein marker data for the subset of the first plurality of cells into pseudo-fluorescent markers data related to the fluorescent intensity data for the subset of the second plurality of cells and output the pseudo-fluorescent markers data for the subset of the cells in the first plurality of cells.
51. The method of any of embodiments 26-50, where the machine learning model is organized in a cascading hierarchical tree structure comprising a plurality of nodes, and wherein each node comprises an individual machine learning model.
52. The method of embodiment 51, wherein each individual machine learning model comprises a neural network model.
53. The method of embodiment 51, wherein each individual machine learning model comprises a gradient boosting tree model.
54. The method of any of embodiments 51-53, wherein the plurality of nodes comprises at least 1000, 1200, 1400, 1600, 1800, 2000, 2200, or 2400 nodes.
55. The method of any of embodiments 26-54, wherein the flow cytometer is configured for at least about 5, at least about 10, at least about 15, at least about 20, at least about 30, at least about 40, at least 50, at least about 60, at least about 70, at least about 80, at least about 90, or at least about 100 fluorescence detection channels.
56. The method of any of embodiments 26-54, wherein the flow cytometer is a full spectrum flow cytometer.
57. The method of any of embodiments 26-56, wherein the single cell transcriptomic value comprises a transcriptional profile for the single cell comprising emergent properties of single cells transcriptomic data.
58. The method of embodiment 57, wherein the emergent properties of single transcriptomic data comprise cell trajectories and/or transcriptional patterns.
59. The method of any of embodiments 26-56, wherein the single cell transcriptomic value comprises emergent properties of a single cell immune cell receptor profile.
60. The method of embodiment 59, wherein the emergent properties comprise immune receptor diversity and/or prediction of clonal expansions.
61. A method for generating a single cell transcriptomic profile for a subject, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cells from the sample; providing only at least a subset of the fluorescent intensity data, or data derived therefrom for the plurality of fluorescently-labeled cells as input to a machine learning model trained using single cell transcriptomics data for a first plurality of cells and pseudo-fluorescent data for the first plurality of cells; generating a predicted single cell transcriptomic value for the fluorescently-labeled cells using the trained machine learning model, thereby generating a single cell transcriptomic profile for the subject.
62. The method of embodiment 61, wherein the pseudo-fluorescent data for the first plurality of cells is generated by matching at least a subset of protein marker data for each cell in the first plurality of cells to fluorescent intensity data for each cell in a second plurality of cells.
63. The method of embodiment 61 or 62, wherein the pseudo-fluorescent data comprises pseudo-fluorescent marker data for the first plurality of cells and/or pseudo-fluorescent cell classifications for the first plurality of cells.
64. The method of embodiment 62 or 63, wherein the matching at least a subset of protein markers comprises transforming the protein marker data into pseudo-fluorescent marker data.
65. The method of embodiment 63 or 64, wherein the pseudo-fluorescent cell classification data are generated by assigning a pseudo-fluorescent cell classification to each cell related to the protein marker data that corresponds to each cell in the first plurality of cells.
66. The method of any of embodiments 61-65, wherein the single cell transcriptomics data for the first plurality of cells is generated using a method for characterizing each cell in the first plurality of cells by simultaneous detection of a plurality of protein marker data and single cell transcriptomic values.
67. The method of embodiment 66, wherein the single cell transcriptomics data comprises a single cell transcriptomic quantification and/or a clonal expansion status.
68. The method of embodiment 67, wherein the clonal expansion status for the first plurality of cells are generated from immune receptor profiling data.
69. The method of embodiment 68, wherein the immune receptor profiling data comprises TCR and/or BCR sequence data.
70. The method of any of embodiments 61-69, where in the single cell transcriptomic profile comprises single cell transcriptomic quantifications for a plurality of genes and/or clonal expansion statuses for a plurality of the fluorescently labeled cells.
71. A method for generating a single cell transcriptomic profile for a subject, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cells from the sample; providing only at least a subset of the fluorescent intensity data, or data derived therefrom for the plurality of fluorescently-labeled cells as input to a machine learning model trained using single cell transcriptomics data for a first plurality of cells and pseudo-fluorescent marker data for the first plurality of cells; generating a predicted single cell transcriptomic quantification for the fluorescently-labeled cells using the trained machine learning model, thereby generating a single cell transcriptomic profile for the subject.
72. A method for generating a single cell transcriptomic profile for a subject, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cells from the sample; providing only at least a subset of the fluorescent intensity data, or data derived therefrom for the plurality of fluorescently-labeled cells as input to a machine learning model trained using single cell transcriptomics data for a first plurality of cells and pseudo-fluorescent cell-classifications for the first plurality of cells; generating a predicted single cell transcriptomic quantification for the fluorescently-labeled cells using the trained machine learning model, thereby generating a single cell transcriptomic profile for the subject.
73. A method for generating a single cell transcriptomic profile for a subject, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cells from the sample; providing only at least a subset of the fluorescent intensity data, or data derived therefrom for the plurality of fluorescently-labeled cells as input to a machine learning model trained using single cell transcriptomics data for a first plurality of cells and pseudo-fluorescent marker data and pseudo-fluorescent cell-classifications for the first plurality of cells; generating a predicted single cell transcriptomic quantification for the fluorescently-labeled cells using the trained machine learning model, thereby generating a single cell transcriptomic profile for the subject. In some embodiments, the single cell transcriptomic data comprises single cell transcriptomic quantifications for a plurality of genes.
74. A method for generating a single cell transcriptomic profile for a subject, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cells from the sample; providing only at least a subset of the fluorescent intensity data, or data derived therefrom for the plurality of fluorescently-labeled cells as input to a machine learning model trained using single cell transcriptomics data, comprising clonal expansion statutes, for a first plurality of cells and pseudo-fluorescent marker data for the first plurality of cells; generating a predicted clonal expansion status for the fluorescently-labeled cells using the trained machine learning model, thereby generating a single cell transcriptomic profile for the subject.
75. A method for generating a single cell transcriptomic profile for a subject, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cells from the sample; providing only at least a subset of the fluorescent intensity data, or data derived therefrom for the plurality of fluorescently-labeled cells as input to a machine learning model trained using single cell transcriptomics data, comprising clonal expansion statutes, for a first plurality of cells and pseudo-fluorescent cell-classifications for the first plurality of cells; generating a predicted a predicted clonal expansion status for the fluorescently-labeled cells using the trained machine learning model, thereby generating a single cell transcriptomic profile for the subject.
76. A method for generating a single cell transcriptomic profile for a subject, comprising; contacting at least a first aliquot of a sample from a subject with at least a first immunophenotyping panel to fluorescently label cells contained within the sample; processing the fluorescently-labeled cell using a flow cytometer to generate fluorescent intensity data, or data derived therefrom, for a plurality of fluorescently-labeled cells from the sample; providing only at least a subset of the fluorescent intensity data, or data derived therefrom for the plurality of fluorescently-labeled cells as input to a machine learning model trained using single cell transcriptomics data, comprising clonal expansion statutes, for a first plurality of cells and pseudo-fluorescent marker data and pseudo-fluorescent cell-classifications for the first plurality of cells; generating a predicted clonal expansion status for the fluorescently-labeled cells using the trained machine learning model, thereby generating a single cell transcriptomic profile for the subject.
77. A method for generating a machine learning model to generate a predicted single cell transcriptomic value, comprising; collecting a population of cells comprising a first plurality of cells and a second plurality of cells; obtaining single cell transcriptomic data and protein marker data for each cell in the first plurality of cells, wherein the single cell transcriptomic data comprise a single cell transcriptomic value for a plurality of genes; obtaining fluorescent intensity data for each cell in the second plurality of cells generated using a flow cytometer to process fluorescently-labeled cells from the second plurality of cells; generating pseudo-fluorescent data by matching at least a subset of the protein marker data for each cell in the first plurality of cells to the fluorescent intensity data for each cell in the second plurality of cells; and training a machine learning model to generate a predicted single cell transcriptomic value, wherein the training is based on the transcriptomic data for at least a subset of the first plurality of cells and the pseudo-fluorescent data for the at least a subset of the first plurality of cells.
78. The method of any of embodiments 61-77, wherein the flow cytometer is a full spectrum flow cytometer.
79. A method for training a machine learning model to generate a predicted single cell transcriptomic value, comprising; collecting a population of cells comprising a first plurality of cells and a second plurality of cells; obtaining single cell transcriptomic data and protein marker data for each cell in the first plurality of cells, wherein the single cell transcriptomic data comprise a single cell transcriptomic value for a plurality of genes; obtaining fluorescent intensity data for each cell in the second plurality of cells generated using a flow cytometer to process fluorescently-labeled cells from the second plurality of cells; generating pseudo-fluorescent data by matching at least a subset of the protein marker data for each cell in the first plurality of cells to the fluorescent intensity data for each cell in the second plurality of cells; and training a machine learning model to generate a predicted single cell transcriptomic value, wherein the training is based on the transcriptomic data for at least a subset of the first plurality of cells and the pseudo-fluorescent data for the at least a subset of the first plurality of cells.
80. The method of embodiment 79, wherein obtaining single cell transcriptomic data and protein marker data for each cell in the first plurality of cells comprises simultaneous detection of a plurality of protein marker data and single cell transcriptomic values.
81. The method of embodiment 79 or 80, wherein the pseudo-fluorescent data comprises pseudo-fluorescent marker data for the first plurality of cells and/or pseudo-fluorescent cell classifications for the first plurality of cells.
82. The method of any of embodiments 79-81, wherein the matching at least a subset of protein markers comprises transforming the protein marker data into pseudo-fluorescent marker data.
83. The method of embodiment 81 or 82, wherein the pseudo-fluorescent cell classification data are generated by assigning a pseudo-fluorescent cell classification to each cell related to the protein marker data that corresponds to each cell in the first plurality of cells.
84. The method of any of embodiments 79-83, wherein the single cell transcriptomic data further comprises clonal expansion statuses for the first plurality of cells.
85. The method of any of embodiments 79-84, wherein the machine learning model has a flexible architecture.
86. The method of any of embodiments 79-85, wherein the single cell transcriptomic values comprise single cell transcriptomic quantifications for the plurality of genes.
87. A method for training a machine learning algorithm to generate a predicted single cell transcriptomic quantification, comprising; collecting a population of cells comprising a first plurality of cells and a second plurality of cells; obtaining single cell transcriptomic data and protein marker data for each cell in the first plurality of cells, wherein the single cell transcriptomic data comprise a single cell transcriptomic quantification for a plurality of genes; obtaining fluorescent intensity data for each cell in the second plurality of cells generated using a flow cytometer to process fluorescently-labeled cells from the second plurality of cells; generating pseudo-fluorescent marker data by matching at least a subset of the protein marker data for each cell in the first plurality of cells to the fluorescent intensity data for each cell in the second plurality of cells; and training a machine learning model to generate a predicted single cell transcriptomic quantification, wherein the training is based on the transcriptomic data for at least a subset of the first plurality of cells and the pseudo-fluorescent marker data for the at least a subset of the first plurality of cells. In some embodiments, the machine learning model has a flexible architecture. In some embodiments, the methods comprise selecting a machine learning model architecture from a group of machine learning architectures based on a distribution of the single cell transcriptomic quantifications for the plurality of genes.
88. A method for training a machine learning algorithm to generate a predicted single cell transcriptomic quantification, comprising; collecting a population of cells comprising a first plurality of cells and a second plurality of cells; obtaining single cell transcriptomic data and protein marker data for each cell in the first plurality of cells, wherein the single cell transcriptomic data comprise a single cell transcriptomic quantification for a plurality of genes; obtaining fluorescent intensity data for each cell in the second plurality of cells generated using a flow cytometer to process fluorescently-labeled cells from the second plurality of cells; generating pseudo-fluorescent cell classifications by matching at least a subset of the protein marker data for each cell in the first plurality of cells to the fluorescent intensity data for each cell in the second plurality of cells; and training a machine learning model to generate a predicted single cell transcriptomic quantification, wherein the training is based on the transcriptomic data for at least a subset of the first plurality of cells and the pseudo-fluorescent cell classifications for the at least a subset of the first plurality of cells. In some embodiments, the machine learning model has a flexible architecture. In some embodiments, the methods comprise selecting a machine learning model architecture from a group of machine learning architectures based on a distribution of the single cell transcriptomic quantifications for the plurality of genes.
89. The method of any of embodiments 86-88, wherein the method comprises selecting a machine learning model architecture from a group of machine learning architectures based on a distribution of the single cell transcriptomic quantifications for the plurality of genes.
90. The method of embodiment 89, wherein the group of machine learning architecture as comprises a hybrid classifier regression multilayer neural network, a regression multilayer neural network, a tweedie regression, a hybrid mean standard error (MSE)/Tweedie neural network and a gradient descent model.
91. The method of any of embodiments 86-90, wherein the machine learning model is trained to predict a single cell transcriptomic quantification from fluorescent intensity data.
92. The method of any of embodiments 89-91, wherein training the machine learning model comprises, minimizing one or more loss functions based on the machine learning model architecture.
93. The method of embodiment 92, wherein the one or more loss functions are selected from a group consisting of a log likelihood loss based on a tweedie distribution, negative log likelihood loss, mean squared error loss, mean absolute error (MAE), and cross entropy loss.
94. The method of any of embodiments 89-93, wherein the predicted single cell transcriptomic value comprises predicted single cell transcriptomic quantifications for the plurality of genes in the second plurality of cells.
95. The method of any of embodiment 79-85, wherein the single cell transcriptomic values comprise clonal expansion statuses.
96. A method for training a machine learning algorithm to generate a predicted single cell transcriptomic quantification, comprising; collecting a population of cells comprising a first plurality of cells and a second plurality of cells; obtaining single cell transcriptomic data and protein marker data for each cell in the first plurality of cells, wherein the single cell transcriptomic data comprise a single cell transcriptomic quantification for a plurality of genes; obtaining fluorescent intensity data for each cell in the second plurality of cells generated using a flow cytometer to process fluorescently-labeled cells from the second plurality of cells; generating pseudo-fluorescent marker data and pseudo-fluorescent cell classifications by matching at least a subset of the protein marker data for each cell in the first plurality of cells to the fluorescent intensity data for each cell in the second plurality of cells; and training a machine learning model to generate a predicted single cell transcriptomic quantification, wherein the training is based on the transcriptomic data for at least a subset of the first plurality of cells and the pseudo-fluorescent marker data and pseudo-fluorescent cell classifications for the at least a subset of the first plurality of cells. In some embodiments, the machine learning model has a flexible architecture.
97. A method for training a machine learning algorithm to generate predicted clonal expansion statuses, comprising; collecting a population of cells comprising a first plurality of cells and a second plurality of cells; obtaining single cell transcriptomic data and protein marker data for each cell in the first plurality of cells, wherein the single cell transcriptomic data comprises single cell clonal expansion statuses for the first plurality of cells; obtaining fluorescent intensity data for each cell in the second plurality of cells generated using a flow cytometer to process fluorescently-labeled cells from the second plurality of cells; generating pseudo-fluorescent marker data by matching at least a subset of the protein marker data for each cell in the first plurality of cells to the fluorescent intensity data for each cell in the second plurality of cells; and training a machine learning model to generate predicted clonal expansion statuses, wherein the training is based on the transcriptomic data for at least a subset of the first plurality of cells and the pseudo-fluorescent marker data for the at least a subset of the first plurality of cells.
98. A method for training a machine learning algorithm to generate a predicted clonal expansion statuses, comprising; collecting a population of cells comprising a first plurality of cells and a second plurality of cells; obtaining single cell transcriptomic data and protein marker data for each cell in the first plurality of cells, wherein the single cell transcriptomic data comprises single cell clonal expansion statuses for the first plurality of cells; obtaining fluorescent intensity data for each cell in the second plurality of cells generated using a flow cytometer to process fluorescently-labeled cells from the second plurality of cells; generating pseudo-fluorescent cell classifications by matching at least a subset of the protein marker data for each cell in the first plurality of cells to the fluorescent intensity data for each cell in the second plurality of cells; and training a machine learning model to generate predicted clonal expansion statuses, wherein the training is based on the transcriptomic data for at least a subset of the first plurality of cells and the pseudo-fluorescent cell classifications for the at least a subset of the first plurality of cells.
99. A method for training a machine learning algorithm to generate a predicted clonal expansion statuses, comprising; collecting a population of cells comprising a first plurality of cells and a second plurality of cells; obtaining single cell transcriptomic data and protein marker data for each cell in the first plurality of cells, wherein the single cell transcriptomic data comprises single cell clonal expansion statuses for the first plurality of cells; obtaining fluorescent intensity data for each cell in the second plurality of cells generated using a flow cytometer to process fluorescently-labeled cells from the second plurality of cells; generating pseudo-fluorescent marker data and pseudo-fluorescent cell classifications by matching at least a subset of the protein marker data for each cell in the first plurality of cells to the fluorescent intensity data for each cell in the second plurality of cells; and training a machine learning model to generate clonal expansion statuses, wherein the training is based on the transcriptomic data for at least a subset of the first plurality of cells and the pseudo-fluorescent marker data and pseudo-fluorescent cell classifications for the at least a subset of the first plurality of cells.
100. The method of any of embodiments 95-99, wherein the machine learning model is a classifier model trained to predict clonal expansion statuses from fluorescence intensity data.
101. The method of embodiment 100, wherein the classifier model is an XGboost classifier.
102. The method of any of embodiment 95-101, wherein the predicted single cell transcriptomic value comprises predicted clonal expansion statuses for the second plurality of cells.

### EXAMPLES

The following examples are included for illustrative purposes only and are not intended to limit the scope of the present disclosure.

### Example 1: Parallel Gating to Identify Cell Classifications

Blood was collected from one healthy individual. PBMCs were prepared from the whole blood and cryopreserved in cryoprotectant in several aliquots. One aliquot was thawed and profiled with flow cytometry. Two fluorescent antibodies panels were used for the flow cytometry, a T panel and an A panel. The fluorescent antibodies in the T panel were, CD16-PerCP-Cy5.5, HLA-DR-BV570, CD56-RY586, CD4-Alexa Fluor 532, CD14-Alexa Fluor 594, TCRgd-PE-Cy7, CD3-BV510, CD8-Spark Violet 538, CD45-PerCP, CD38-BUV805, PD-1/CD279-BV421, CD5-BUV496, CD19-PE-Cy7, ICOS/CD278-BV750, CD25 (clone 2A3)-PE, CD25 (clone M-A251)-PE, LAG-3/CD223-BB790-P, CD161/KLRB1-BB755-P, CD122/IL2RB-BB630-P2, TCR Vα7.2-VioBlue, TCR Vα24-Ja18-PE/Dazzle™ 594, CD45RO-APC, CD95-BV480, CD127-PE-Cy5.5, TIM-3/CD366-R718, KLRG1-PE-Fire 810, CD27-APC/Fire 810, CD28-BUV563, CD45RA-APC/Fire 750, CD31-BV605, CD57-FITC, CD39-BUV661, TIGIT-BUV395, CD103/ITGAE-BB660-P2, TCR Vδ2-PerCP-Vio700, TCR Vδ1-VioBlue, CCR4/CD194-BVδ50, CXCR3/CD183-PE-Cy5, CCR7/CD197-BV786, CCR6/CD196-BV711, CCR10-BB700, CXCR5/CD185-BUVδ15. The fluorescent antibodies in the A panel were CD16-PerCP-Cy5.5, HLA-DR-BV570, CD56-RY586, CD4-Alexa Fluor 532, CD14-Alexa Fluor 594, TCRgd-PE-Cy7, CD3-BV510, CD8-Spark Violet 538, CD45-PerCP, CD38-BUV805, PD-1/CD279-BV421, CD5-BUV496, CD19-PE-Cy7, CD319-PE-Dazzle594, CD138-Alexa Fluor 647, CD10-PE-Cy5, CD294/CRTH2-BUVδ15, CD303/BDCA-2/CLEC4C-APC/Fire 750, IgM-BUV395, IgD-PerCP-eFluor 710, CD337/NKp30-BV711, CD24-BVδ05, CD40-BVδ50, CD141-BV786, TACI/CD267-PE, CD69-VioBlue, CD27-PE/Fire 810, CD11c-BV480, PD-L1/CD274-BB660-P2, CD86-APC-R700, CD335/NKp46-BB630-P2, CD43-BB790-P, CD62L-BUV563, CD123-BV750, CD1c-Alexa Fluor 488.

Separately, another aliquot was thawed, performed CITE-seq by staining the cells with TotalSeqC barcoded antibodies directed to CD16, HLA-DR, CD56, CD4, CD14, TCRgd, CD3, CD8, CD45, CD38, PD1, CD5, CD19, CD319, CD138, CD10, CRTH2, CD303, IgM, IgD, CD337, CD24, CD40, CD141, CD267, CD69, CD27, CD11c, CD274, CD86.1, CD335, CD43, CD62L, CD123, CD1c, CD278, CD25, CD223, CD161, CD122, TCR Va7.2, TCR Va24-Ja18, CD45RO, CD95, CD127, CD366, KLRG1.1, CD28.1, CD45RA, CD31, CD57, CD39, TIGIT, CD103, TCR Vd2, CD194, CD183, CD197, CD196, CCR10, and CD185, then sequenced by a 10× Genomics Platform. In total, 9701 cells were sequenced. ADT library was sequenced to a depth of 10,000 raw reads/cell and the mRNA gene expression library was sequences to a depth of 40,000 raw reads/cell with the 10× Genomics 5′ immune receptor profiling and feature barcoding kit. Count matrices were QC filtered by standard RNA and ADT metrics, normalized, and then processed ADT count matrices were exported as FCs files. The steps were performed using the Seurat package of R within a Dagster data pipeline. After standard filtering and QC, 8658 cells were analyzed.

Fluorescence signals from flow cytometry and ADT expression signals from matched antibody markers must be harmonized. This example provides an illustration of the harmonization by the generation of pseudo-fluorescent classifications. ADT data was converted into FCS format by a bespoke workflow following processing using a standard set of tools built for analyzing CITE-seq data, including CellRanger and the R Seurat library, and gated in a parallel gating hierarchy to the flow cytometry data to generate pseudo-fluorescent classifications for the cells processed with CITE-Seq. The flow cytometry and ADT marker values were derived from the same individual sample.

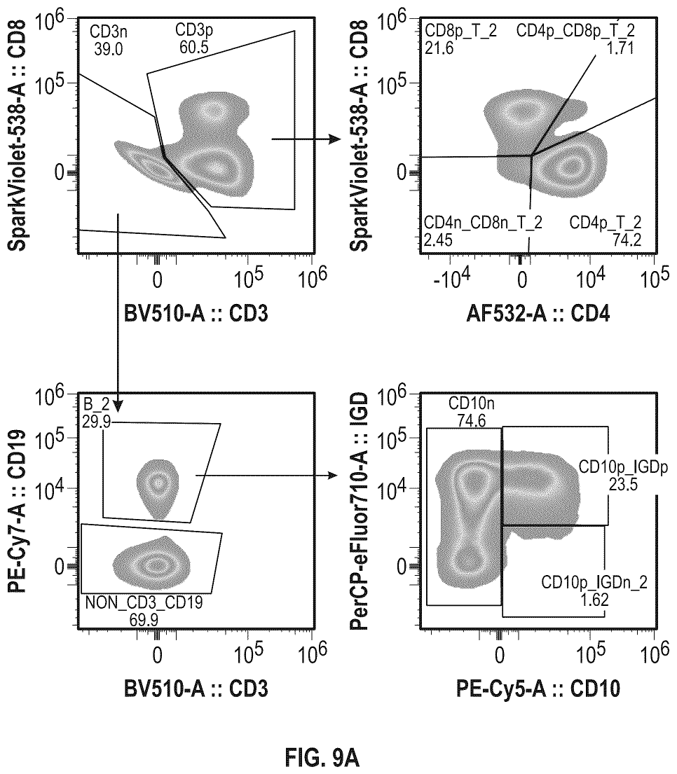

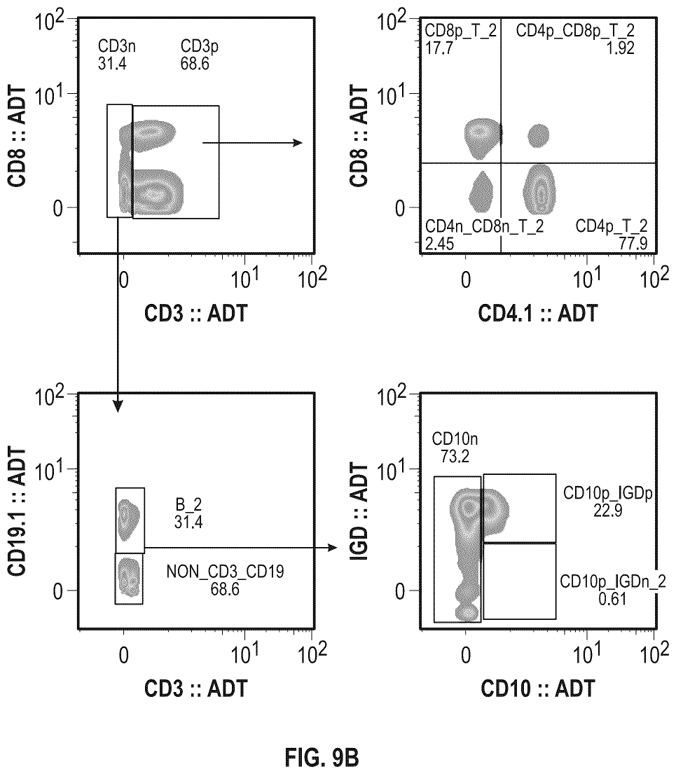

FIG. 9A shows example flow cytometry classifications from fluorescent markers. FIG. 9B shows example pseudo-flow classifications from ADT markers. The ADT marker data have been converted into FCS format. Parallel gating revealed equivalent cell classifications (e.g. CD10p_IGDp B cells) at similar frequencies using matched antibody marker channels for flow cytometry and CITE-Seq (e.g. BV510-CD3 and CD3::ADT).

### Example 2: Alignment of Cell Classification Frequencies Derived from Flow Cytometry and ADT Expression

This example demonstrates successful parallel cell classification across data derived from flow cytometry and from CITE-seq. This example further provides an illustration of successful generation of pseudo-fluorescent classifications that can be used as training data for the machine learning models described herein.

Blood samples from 9 donors were split and processed by flow cytometry and by CITE-Seq exactly as for Example 1 (i.e. Example 1 shows results from one of the donors of this experiment). Flow cytometry was performed with antibodies listed above and acquired on a Sony ID7000. FCS files were manually gated in FlowJo and population ratios and counts exported. CITE-Seq data off from the 10× platform (described above for antibodies and sequencing parameters) was processed in CellRanger to map reads to the human genome and quantify gene expression, to the antibody barcode sequences and pre-process the dataset to filter out empty droplets. In total, between 7824 and 15956 cells were sequenced per donor. Subsequently, count matrices were QC filtered by standard RNA and ADT metrics, normalized, and then processed ADT count matrices were exported as FCS files—these steps were performed using the Seurat package of R within a Dagster data pipeline. Between 7234 and 10879 cells were analyzed per donor after QC filtering. ADT FCS files were then manually gated in FlowJo (parallel to the flow cytometry), ratios and counts of populations were exported. Together, the counts and ratios of populations were aligned and correlated in R.

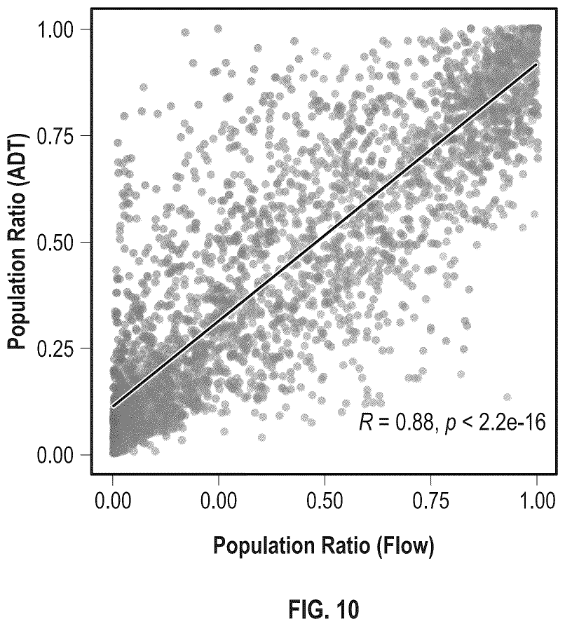

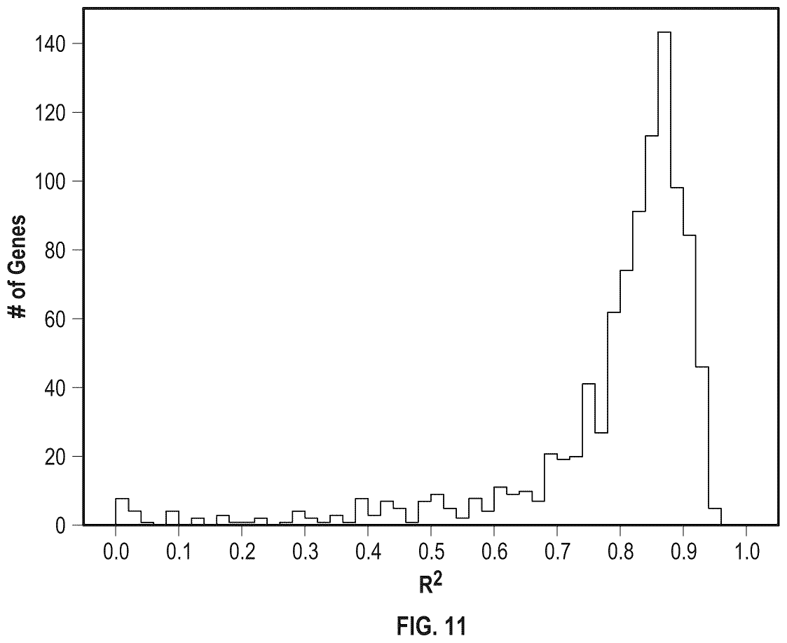

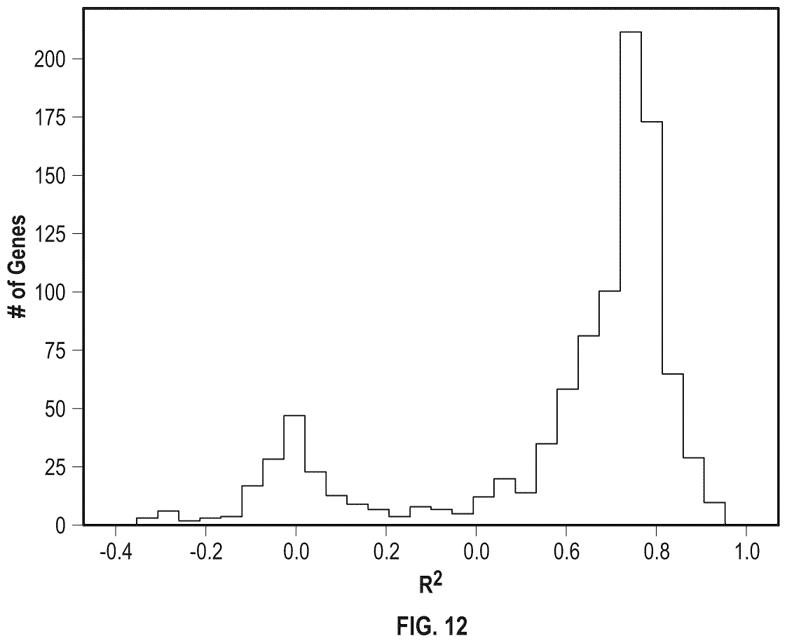

Classifications were filtered to include those containing more than 22 cells to ensure only statistically reliable populations were included. FIG. 10 shows the correlation between the flow cell classification population ratios for each cell type (x-axis) and the pseudo-fluorescent classification population ratios for each cell type (y-axis) in each sample. The line and statistics represent Pearson's correlation between the results of both methods.

### Example 3: Prediction of Single Cell Transcriptomic Values Using ADT Marker Values

This example demonstrates successful prediction of single cell transcriptomic data for 1000 genes using ADT marker values collected from 9 donors using CITE-Seq. The data were collected and prepared according to the methods described in Example 1 and Example 2.

A fully connected neural network consisting of 1 input node per marker (33 nodes), fully connected to a hidden layer consisting of 20 nodes, fully connected to a single output node representing gene expression was created for every gene and was used to generate a set of predictions which combined to produce a transcriptomic profile.

Training was performed with data from 7 samples and the 2 held out samples were used for validation. The training and validation was repeated according to a random subsampling of 7 samples for training and 2 for testing.

FIG. 11 shows the correlation between the predicted transcriptomic value and the actual expression value measured by CITE-Seq for the top 1000 most variable genes as determined by variance stabilizing transformation.

### Example 4: Prediction of Single Cell Transcriptomic Values Using ADT Cell Classification

This example demonstrates successful prediction of single cell transcriptomic data for 1000 gene using ADT cell classifications derived from ADT marker data collected from 9 donors using CITE-Seq. The data were collected and prepared according to the methods described in Example 1 and Example 2.

A fully connected neural network with one hidden layer was used for prediction. A sparse autoencoder was utilized to reduce the dimensionality of the classification to 64 dimensions. Prior to input, the ADT cell classifications were filtered to remove low confidence classifications according to the method described in Example 2.

Training was performed with data from 7 samples and the 2 held out samples were used for validation. The training and validation was repeated according to a random subsampling of 7 samples for training and 2 for testing.

FIG. 11 shows the correlation between the predicted transcriptomic value and the actual expression value measured by CITE-Seq for the top 1000 most variable genes as determined by variance stabilizing transformation.

As ADT cell classifications are inherently equivalent to pseudo-fluorescent marker classifications, this represents a preliminary manifestation of the machine learning models described herein.

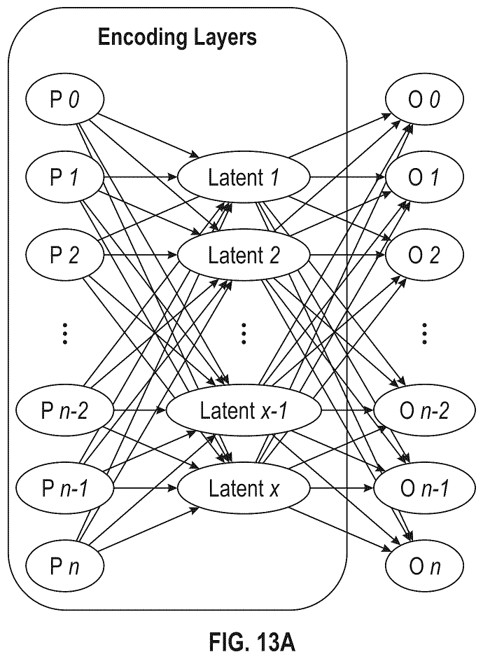

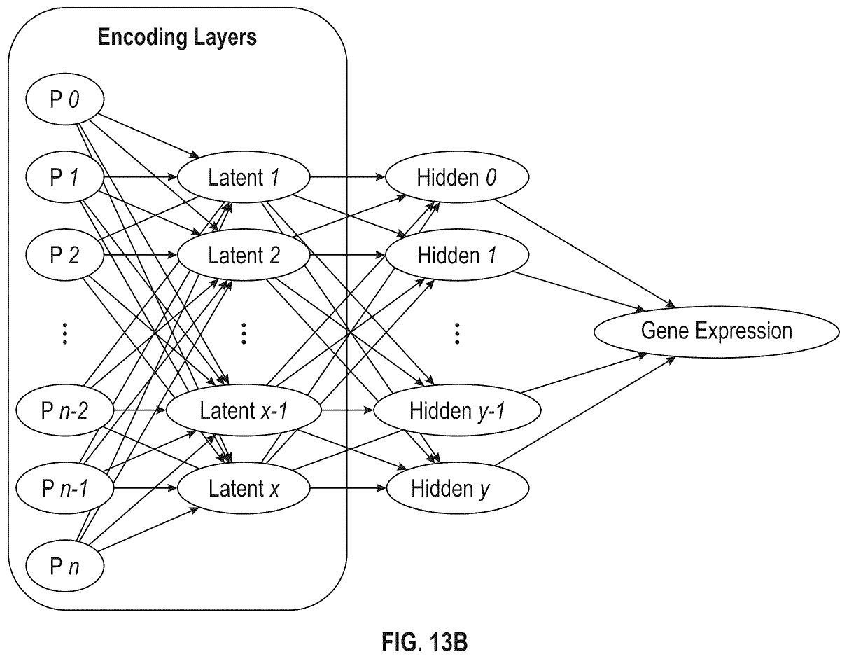

FIG. 13A demonstrates an exemplary architecture comprising one latent layer. The network is trained such that latent features in the data are encoded in the latent layer.

FIG. 13B demonstrates an exemplary architecture wherein the input and latent layer weights were then used as the first layers in another neural network and fine-tuned to predict gene expression for the event.

The exemplary model was trained once per gene.

### Example 5: Training a Gradient Descent Model to Predict Gene Expression

The example demonstrates training a gradient descent ML model to predict transcriptomic information, i.e., gene expression, from ADT marker data collected from 9 donors using CITE-Seq. The data were collected and prepared according to the methods described in Example 1 and Example 2.

A gradient descent model with 150 estimators was trained, one per gene, on the ADT marker input data, one input per marker, and a train/test split of 80%/20% sampled randomly from 7 biological samples was used to evaluate the model until it stopped learning. This collection of models was then used to predict gene expression values validated using a hold out set of 2 samples.

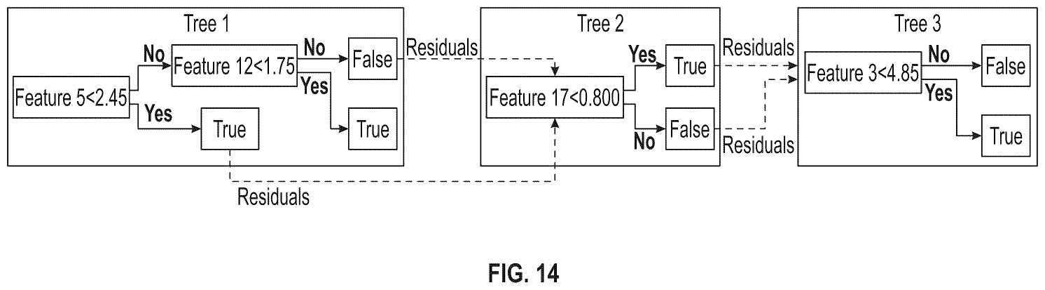

FIG. 14 demonstrates an example collection of decision trees that together make up a gradient descent ML model where the residuals, or error, from each tree cascades to the next in sequence with the final tree producing the predicted value.

### Example 6: Generating Training Data for 50 Individuals

This example demonstrates the preparation of training data for the machine learning model is described herein. A training data set comprising 50 individuals was generated. For each individual sample, flow cytometry and single cell transcriptomic data was generated. The single cell transcriptomic data included single cell gene expression, protein marker data (ADT data) and immune repertoire information collected using CITE-seq. Pseudo-fluorescent marker data and pseudo cell-classifications were generated in order to harmonize the flow data with the single cell transcriptomic data for training of machine learning models.

A first sample of cryopreserved PBMCs from 50 individuals were profiled in triplicate using the T panel and A panel of antibodies according to Table 2 and a viability dye. High throughput flow cytometry with full spectrum flow cytometry (FSFC) ML-based cell classification according to the methods described in U.S. Ser. No. 18/353,022, and corresponding U.S. Patent publication US2024-0192210-A1, hereby incorporated by reference in its entirety, was used. The classification model was trained on 10 manually gated samples. The remaining samples were input into the trained classification model to predict population membership for all cells in the sample.

A second sample of the cryopreserved PBMCs from the individuals, as well as samples from 3 of the 9 individuals in Example 1 were subjected to the CITE-seq protocol. (Total training data was 53 individuals). This version of the CITE-seq protocol included the module for creating a library to capture immune repertoire information (e.g. TCR and BCR libraries). The library preparations were performed according to the manufacturing instructions.

Cell Ranger processing of raw sequencing data was performed. Filtered count matrices were then processed using an orchestration platform (e.g. Dagster) to execute an R script within a docker container. scRNAseq QC was performed in Seurat, removing low-quality cells, including those with low sequencing depth, evidence of cell death, red blood cells, platelets, and potential doublets. This resulted in a high-quality dataset containing 872,101 cells from 53 individuals. Gene expression data was log 1p-normalized and further QC performed on a sketch of 200,000 cells. PCA was performed on scaled, variable features and the top 50 PCs employed in downstream UMAP and clustering. ADT data (protein marker data) was normalized using center log normalization.

Because TCR-Vd1 antibody for protein marker feature barcoding was not commercially available, expression of the corresponding gene segment TRDV1 was exported as a substitute channel. Every sample produced two files, each containing appropriate protein marker channels matching the FSFC A and T panels, respectively. Additionally, the TCR sequences were read and filtered using scRepertoire and receptor clones were quantified at the amino acid level (additional information in Example 11).

A key challenge associated with the prediction of single cell gene expression data from FSFC data is that there is no per-cell registration across the two technologies (Flow cytometry and single cell transcriptomic data) when a blood sample is split and run in parallel in FSFC and scRNAseq, no single cell is profiled by both technologies. The methods described herein, circumvent this challenge by generating pseudo-fluorescent data such as pseudo-fluorescent marker data and/or pseudo-fluorescent cell classifications from the transcriptomic data. Cell identities between the FSFC protein expression data and scRNAseq gene expression data were matched taking advantage of the identical set of protein makers profiled in the FSFC and were profiled in FSFC (fluorescence signals) and feature barcoded in CITE-seq (ADT/protein signals and immune repertoire characterizations). Alignment of protein expression distributions and/or annotation with common immune cell population classifications derived from these overlaps allowed models to be trained with protein signals from CITE-seq then validly deployed on fluoresce signals from FSFC as described herein.

Generating pseudo-fluorescent data such as pseudo-fluorescent marker data and/or pseudo-fluorescent cell classifications required aligning the raw signal distributions for matching markers because while feature barcoding (ADT marker data) in CITE-seq and FSFC were used to profile the same set of protein markers. The techniques employed to measure these expression levels were fundamentally different and therefore resulted in misaligned signal raw distributions.

The difference in distribution may have been due to the lower sensitivity in ADT marker measurements from CITE-seq compared to the fluorescence measurements in FSFC which resulting the presence of zeros in the ADT data that did not appear in the FSFC data.

Harmonization methods were used to correct for mismatch in feature distribution thereby to create the pseudo-fluorescent marker data. The method included adjusting the protein expression values for single cells from feature barcoding in the ADT data to the reference matched FSFC sample. In effect, this converted the single cell sequencing-derived protein expression data (ADT marker data) into pseudo-fluorescent marker data compatible with the parallel FSFC data.

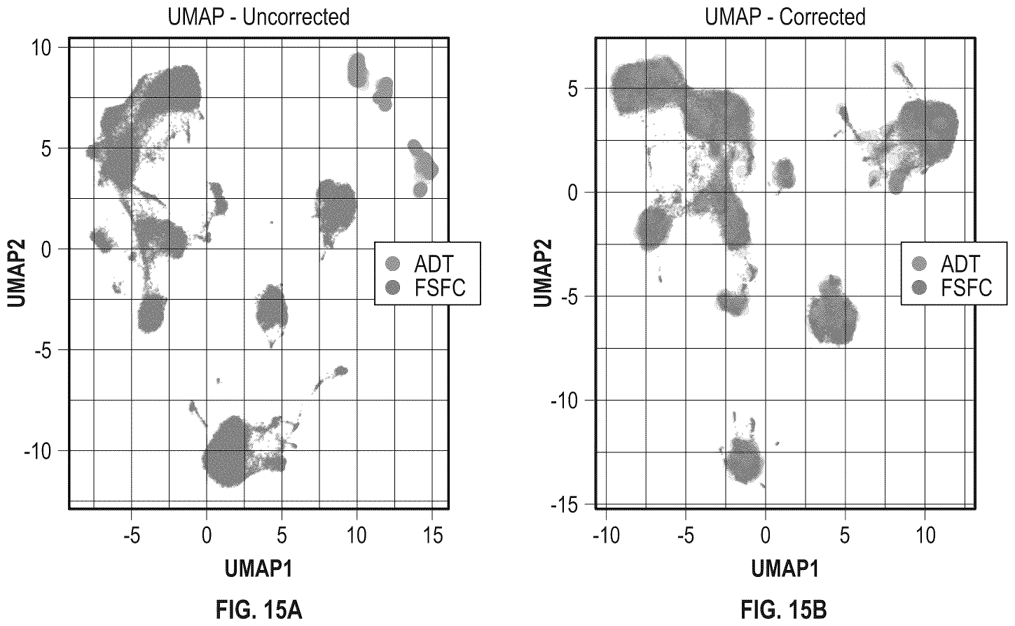

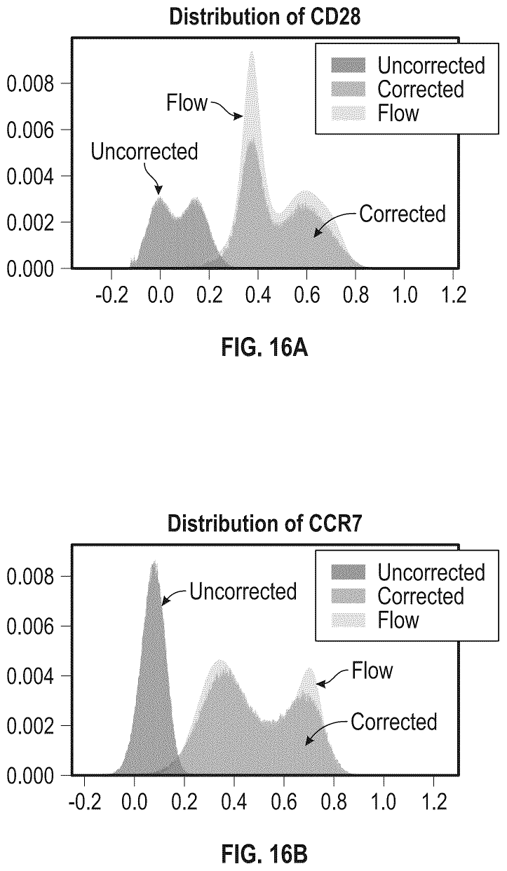

An empirical Bayes-based batch correction method from the publicly available cyCombine package was used to transform the ADT protein marker distributions to align with reference, sample-matched FSFC fluorescence data filtered for viable cells. FIG. 15A shows a UMAP representation of the matched sample ADT and FSFC data before the harmonization and FIG. 15B shows a UMAP representation of the same samples after harmonization. As shown in FIGS. 16A and 16B the distribution of uncorrected ADT measurements and flow measurements for CD28 and CCR7 (respectively) did not overlap, but after the correction the distributions for the markers did.

Machine learning cell classification as described in U.S. Ser. No. 18/353,022, and corresponding U.S. Patent publication US2024-0192210-A1, was used to annotate individual cells with immune population memberships based upon combinatorial fluorescence signal intensities from the FSFC. The structure of the gating hierarchy reflected expert biological annotation of known cell lineages, subsets and phenotypes.

Once memberships were annotated, cell-population level summary data for each of the 53 samples, including total count, ratio to a defined cell population, and mean fluorescence intensity of all markers was calculated.

Pseudo-fluorescent cell classifications were generated for the sample with transcriptomic data by mapping the pseudo-fluorescent marker data using parallel gating of the pseudo-fluorescent marker data and the FSFC population classification. Cell population level summary data was calculated using the pseudofluorescence to generate sample matched total cell counts, ratios to the defined cell population, and mean fluorescence intensity of all markers.

The training data was filtered to improve reliability of the training data. Pseudo Cell classifications (pseudo-fluorescent cell classifications) were selected based upon statistical reliability (sufficient mean cell counts in pseudofluorescence and flow datasets) and correlation across all matched training samples. Populations with a mean cell count of less than 15 were removed. In the maintained populations, 75% of samples fell within 30% standard deviation of y=x correlation OR Spearman R>0.5 and root mean square error of <0.2.

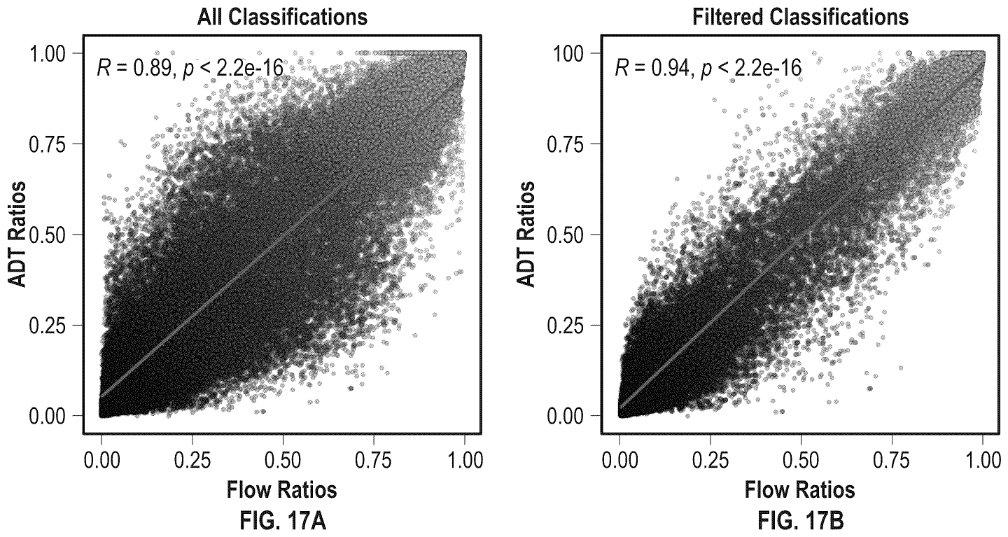

The filtering resulted in 786 pseudo-fluorescent cell classifications. FIG. 17A shows per population, per sample correlations across pseudo-fluorescent cell classification (generated from ADT markers) and FSFC before filtering of the cell classifications and FIG. 17B shows the same data after filtering.

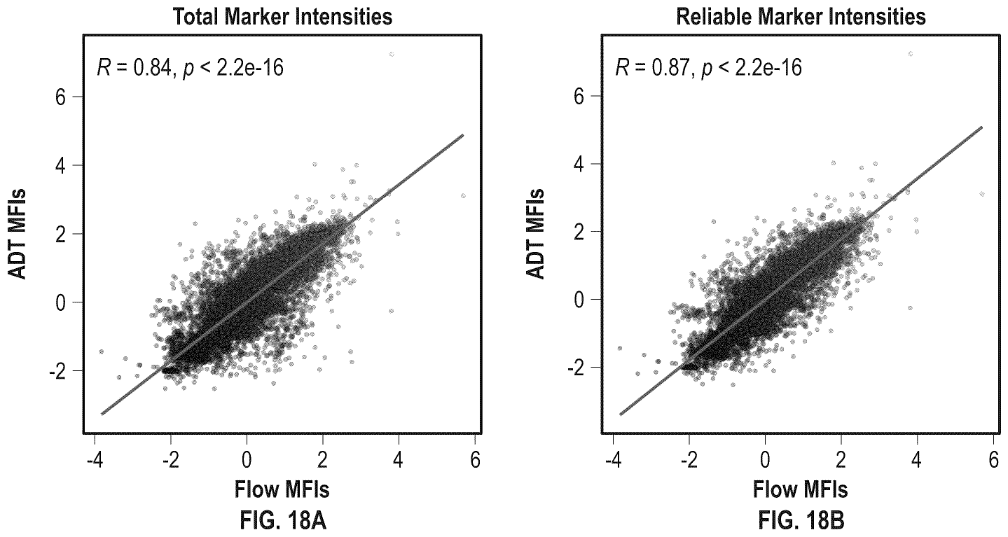

Pseudo-fluorescent marker data intensities were also filtered based upon correlation across all matched training samples. Pseudo-fluorescent markers passing the filter were defined by comparison of scaled mean fluorescence intensities of all markers within several high order cell populations (e.g. B cells, T cells, monocytes) from pseudo fluorescent and fluorescence data from matched training samples. Markers CD69, CD294 and TCR-Va24-Ja18 were filtered out. FIG. 18A shows the correlation between the pseudo-fluorescent marker data from the ADT markers and fluorescence from the FSFC data for all markers. FIG. 18B shows the same correlation after filtering.

As shown in subsequent examples, machine learning models were trained and employed to predict single cell gene expression values from FSFC fluorescence data. A variety of inputs, model architectures, loss functions and evaluation metrics were experimented with.

### Example 7: Training a Machine Learning Model with Highly Expressed Genes

This example demonstrates the training of a hybrid classifier regression neural network with 364 highly expressed genes from the training data generated in Example 6.

A set of 364 highly expressed genes defined as mean expression of greater than 0.5 normalized counts in the measured scRNAseq data, was formed by selecting highly expressed genes from the markers genes of main immune population clusters and a set of common biological pathways. Normalized counts were generated by dividing the transcript count for a gene by the total transcript counts for the cell, multiplying the value by 10,000 and then natural-log transforming the value using log 1p. The genes from the common biological pathways were included to validate the method without the complexity resulting from dropouts and rare genes. The 364 genes were modelled with a normal distribution and a binary distribution to handle zeros.

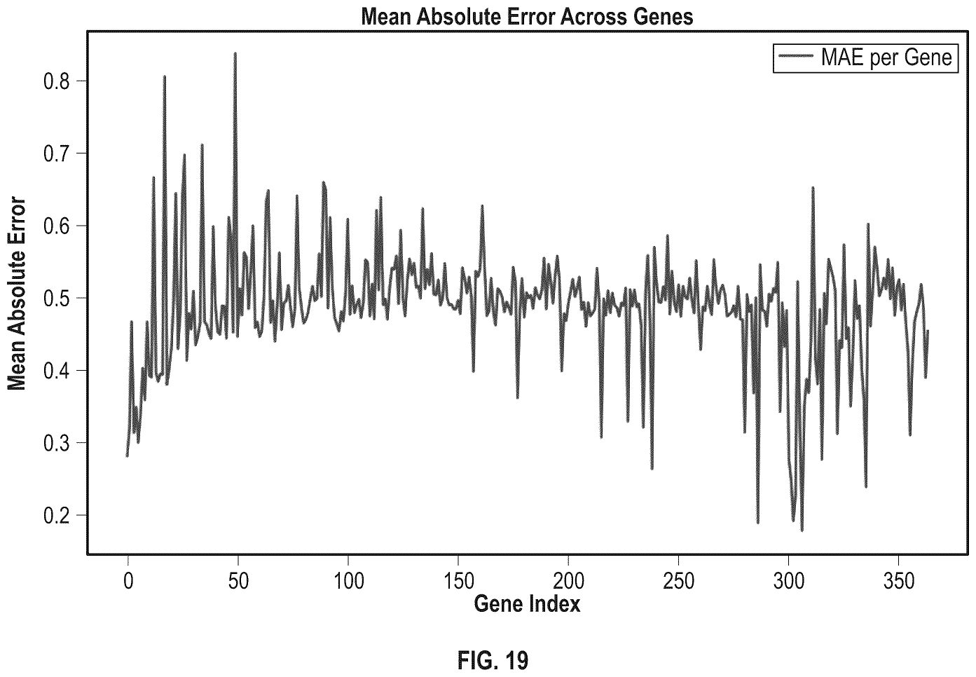

A multilayer neural network was trained to predict the highly expressed genes. The model was trained on all pseudo-fluorescent markers intensities (A+T panel combined), employing mean square error loss function. FIG. 19 shows an evaluation of prediction of single cell gene expression for the highly expressed genes based on training of the neural network with pseudo-fluorescent marker data and the gene expression measurements for the highly expressed genes. The gene expression predictions were validated by comparing the gene expression predictions from the pseudo-fluorescent data to the measured gene expression measurements.

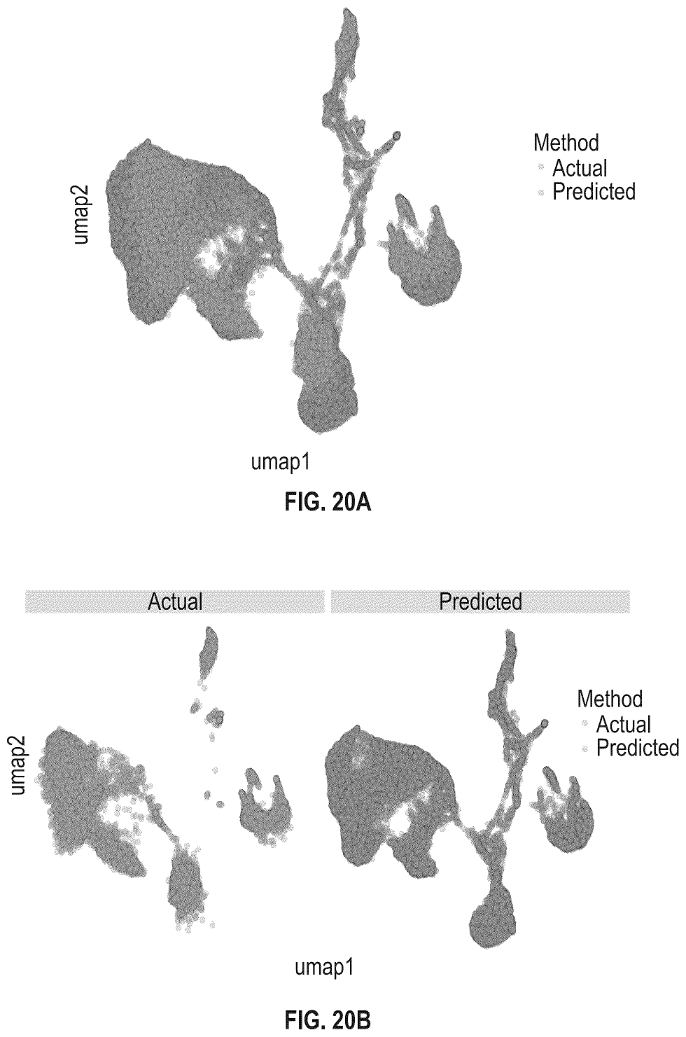

FIG. 20A-20B shows the actual (measured) gene expression and predicted gene expression for the highly expressed genes in UMAP space following performance of batch correction (FIG. 20A overlapping data, FIG. 20B side by side data).

The neural network model served to demonstrate the associations between surface protein signals and gene expression.

A hybrid neural network with a binary classifier and regressor was trained (separately) on A and T panel pseudo-fluorescent marker data intensities and the set of 364 highly expressed genes. The models outputted a continuous output and a binary (zero or non-zero) output for each gene. A mean squared error loss function was used for the continuous output and cross entropy loss was used for the binary output The binary output was multiplied by the continuous output so that the predicted non expressed values became zero and the predicted expressed values remain the continuous value.

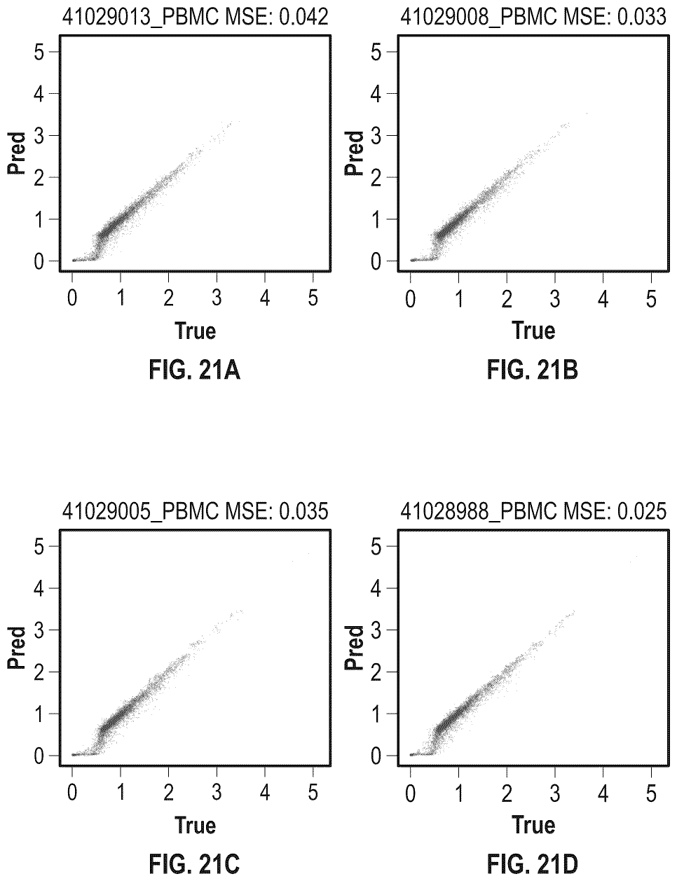

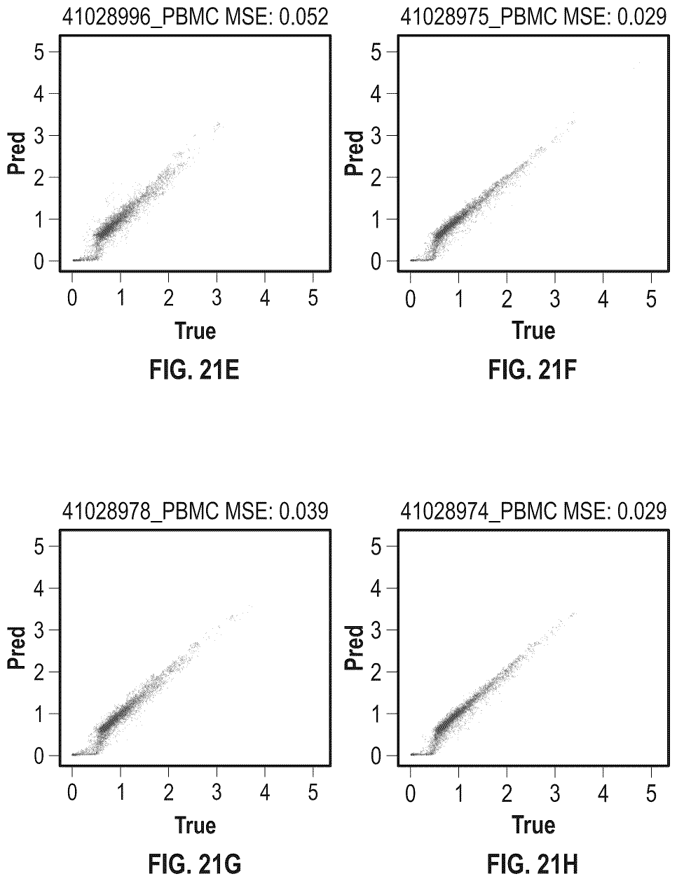

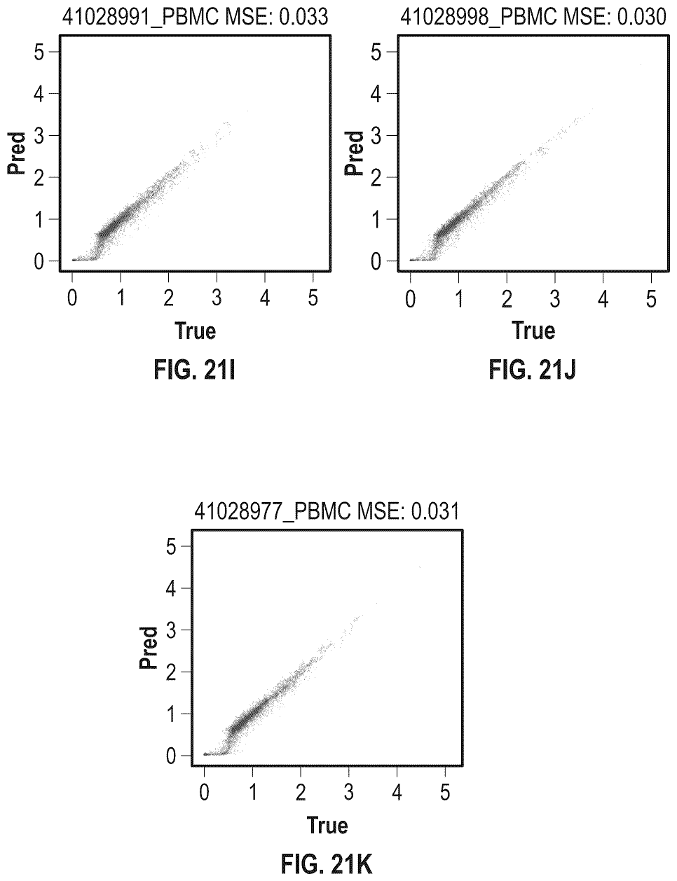

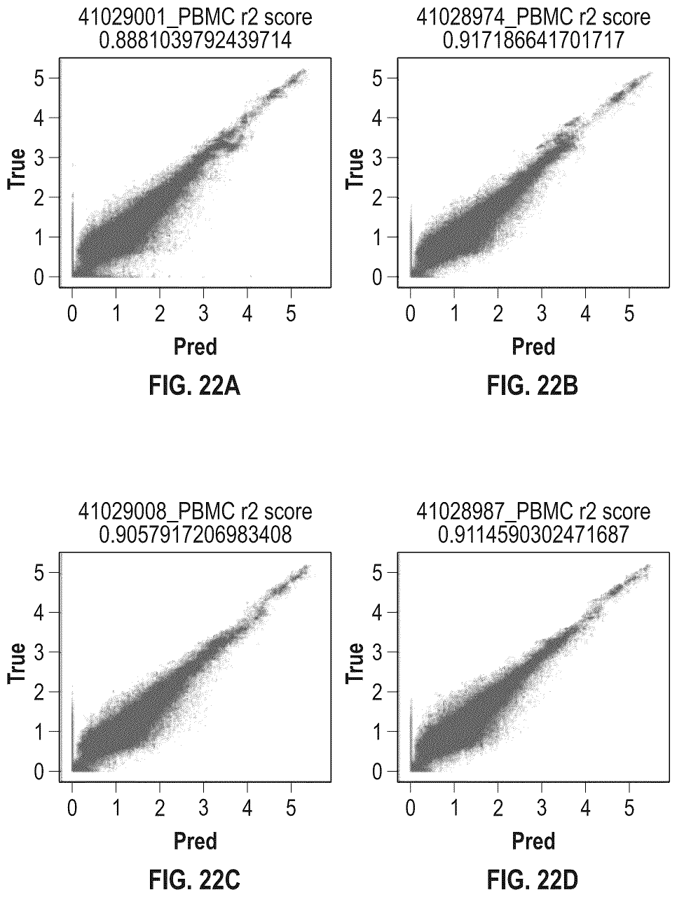

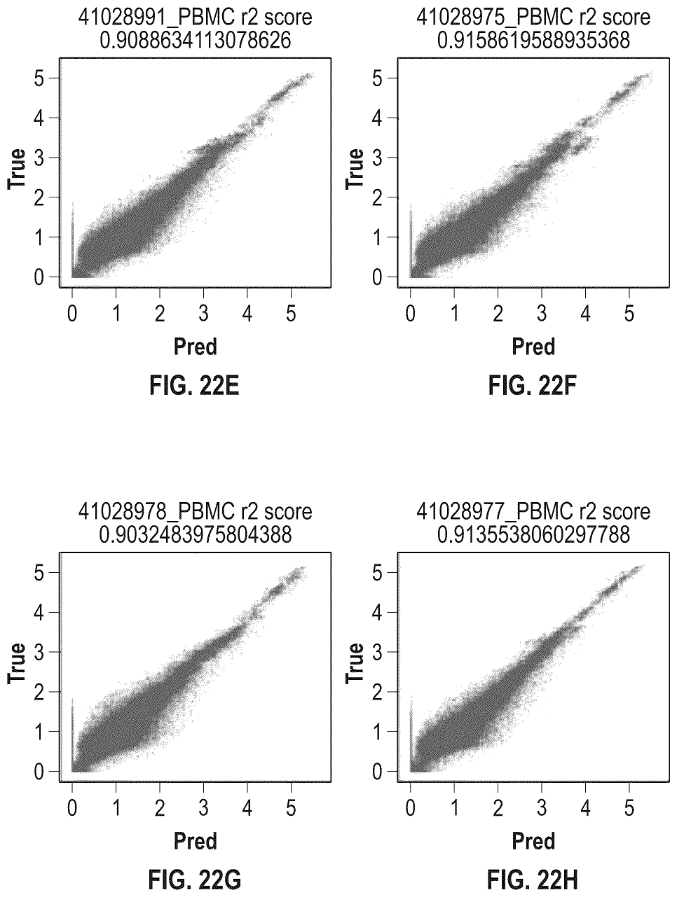

FIG. 21 shows evaluations of the prediction of 364 highly expressed genes from FSFC fluorescence signal intensities (T panel) compared to ground truth single cell gene expression using the hybrid classifier/regressor neural network trained with the pseudo fluorescent marker data. Each panel is a sample and MSE annotations represent mean squared error evaluation of each gene mean per broad cell lineage. FIG. 22 shows evaluations of the prediction of the 364 highly expressed genes from the FSFC fluorescence signal intensities (T panel) compared to ground truth single cell gene expression using hybrid classifier/regressor neural network. Each panel is a sample and MSE annotations represent mean absolute error evaluation of each gene mean per FSFC-defined cell classification membership (for all matched pseudo fluorescent classification, filtered for MAE<0.1).

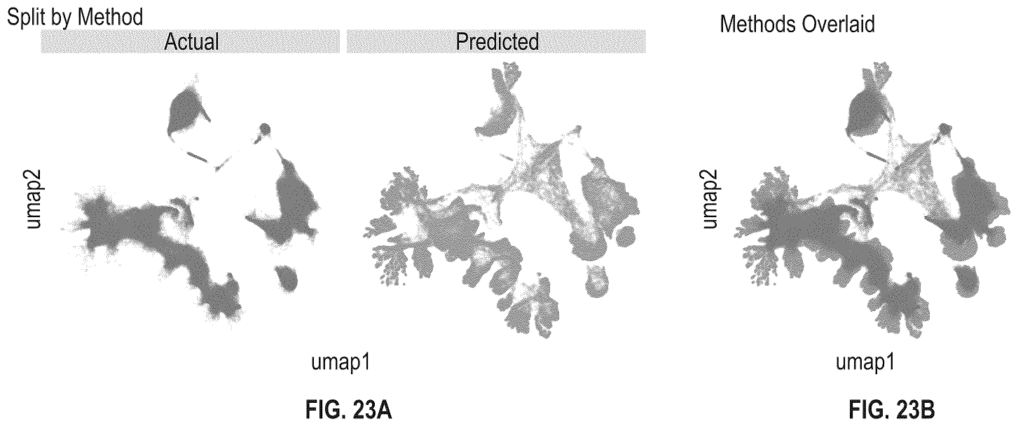

FIG. 23A-23B shows the actual gene expression and predicted gene expression for the 364 highly expressed genes in UMAP space following batch correction (FIG. 23A side by side FIG. 23B overlapping data).

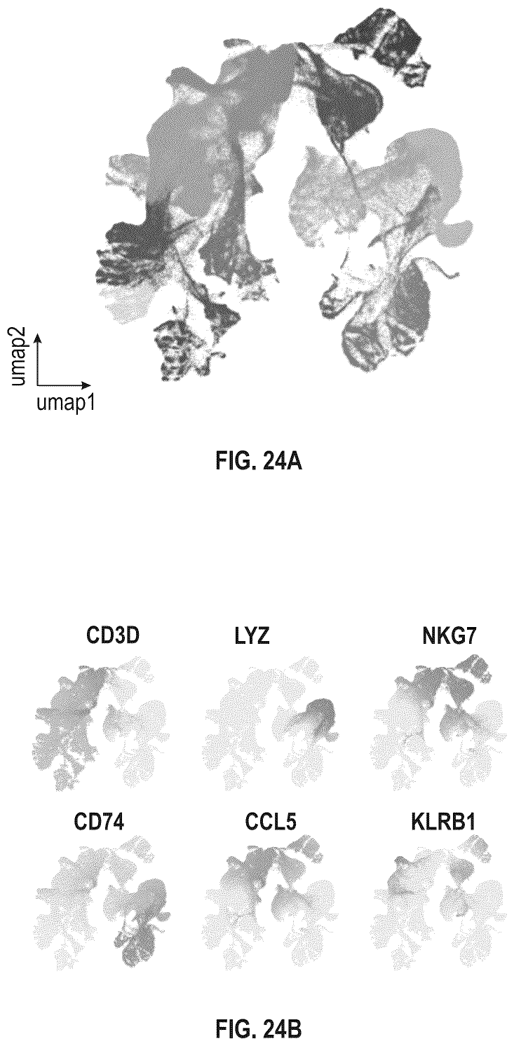

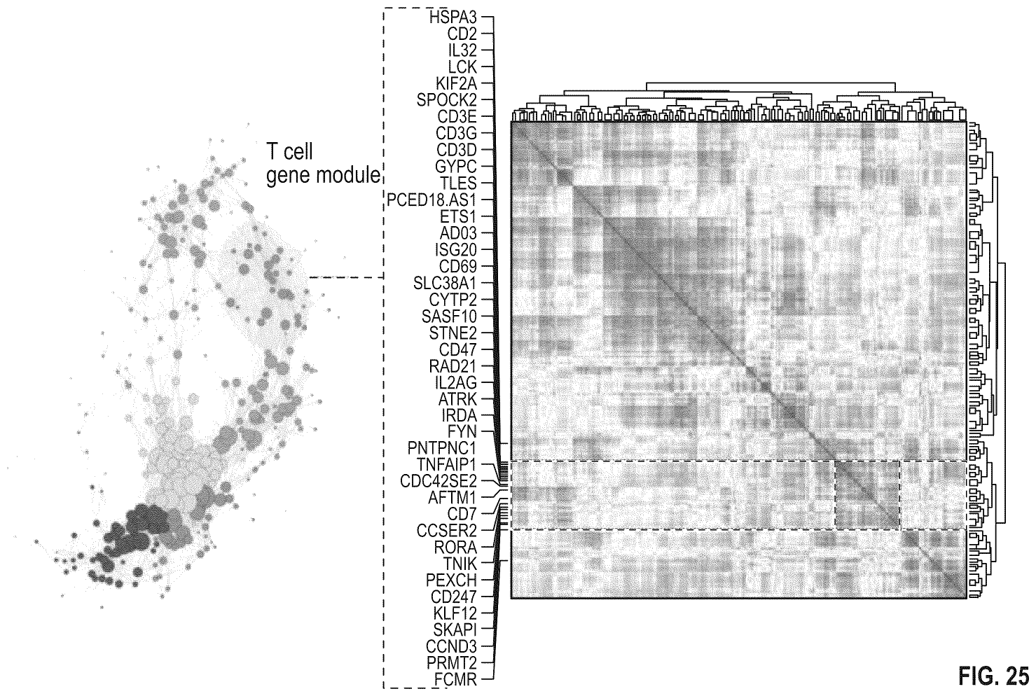

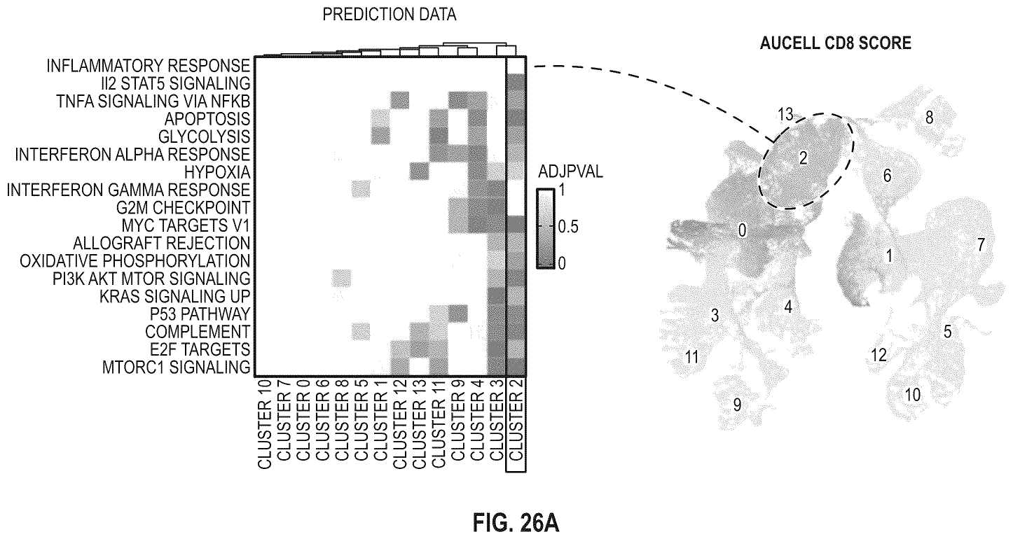

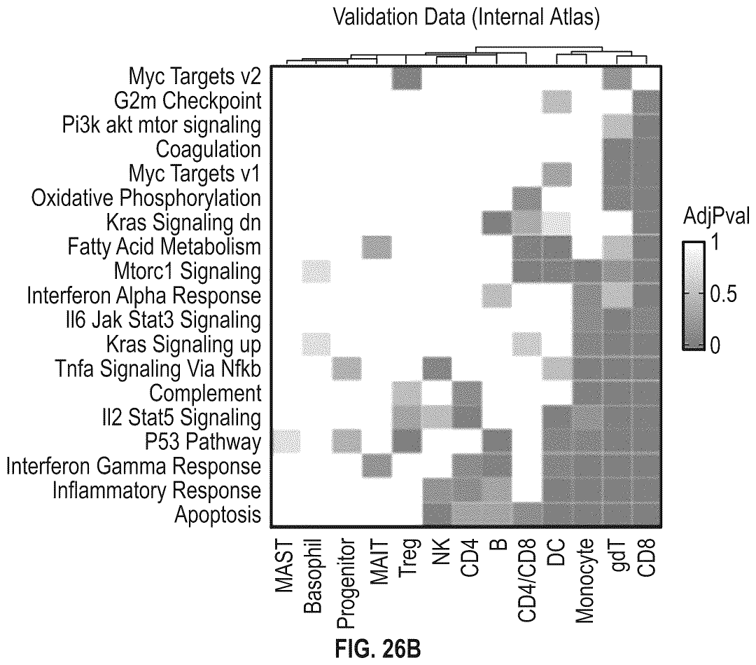

The hybrid classifier/regressor neural network model was also deployed on a separate FSFC dataset comprising 1,048 FSFC samples. This showed the ability to routinely and easily profile very large cohorts of samples, enabling appropriately powered population-scale immunophenotyping. FIG. 24A shows clustering of the cells using the predicted single cell gene expression in UMAP space. FIG. 24B shows the cells clustered by expected marker genes. In the predicted data, expected gene network modules correlated with each other. FIG. 25 shows clustering of the T cell gene module in a correlation heat map. Pathway analysis was performed across all identified cell clusters using the predicted measurements. Donors were stratified into lower (<30 years old) and upper (50+ years old) age bins, then differences between age bins was calculated for every cell type. Any pathway that changed significantly was defined as an age-associated pathway. The analysis showed the strongest age associated pathway changes in a cluster annotated as CD 8T cells (FIG. 26A). The pathway analysis was validated using measured single cell gene expression data (FIG. 26B).

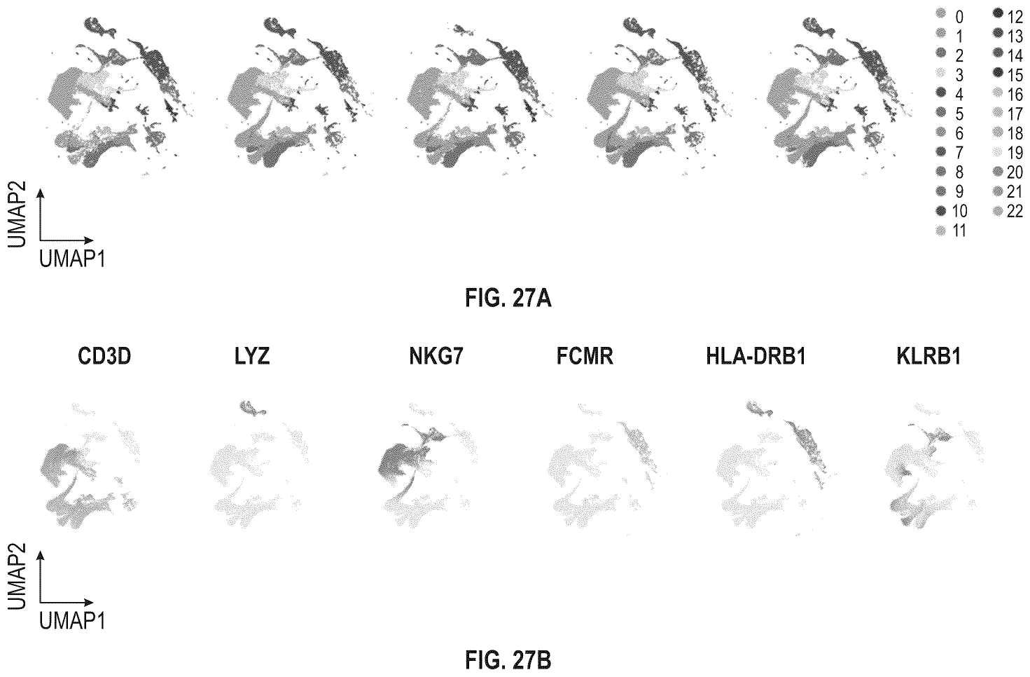

The hybrid classifier/regressor neural network model was also deployed on archival biobank FSFC samples to predict gene expression for the 364 genes. These samples represented low blood volume/fragile samples. Traditional scRNAseq methods would have suffered from substantial cell loss or failure due to the sample quality. UMAP and clustering analysis of five samples identified anticipated immune population structures including donor-to-donor heterogeneity (FIG. 27A). The cells and each sample clustered according to expression of known population marker genes (FIG. 27B).

### Example 8: Training a Machine Learning Model with Variable Genes

This example demonstrates the training of neural networks with 2000 variable genes from the training data generated in Example 6. This example demonstrates the ability to predict gene expression for genes with significant variation, but low expression counts.

A set of 2000 most variable genes were selected. The genes in the most variable genes had a large proportion of zeros. The genes were modeled using a tweedie distribution with k=1.5 combined with a binary distribution as the base model.

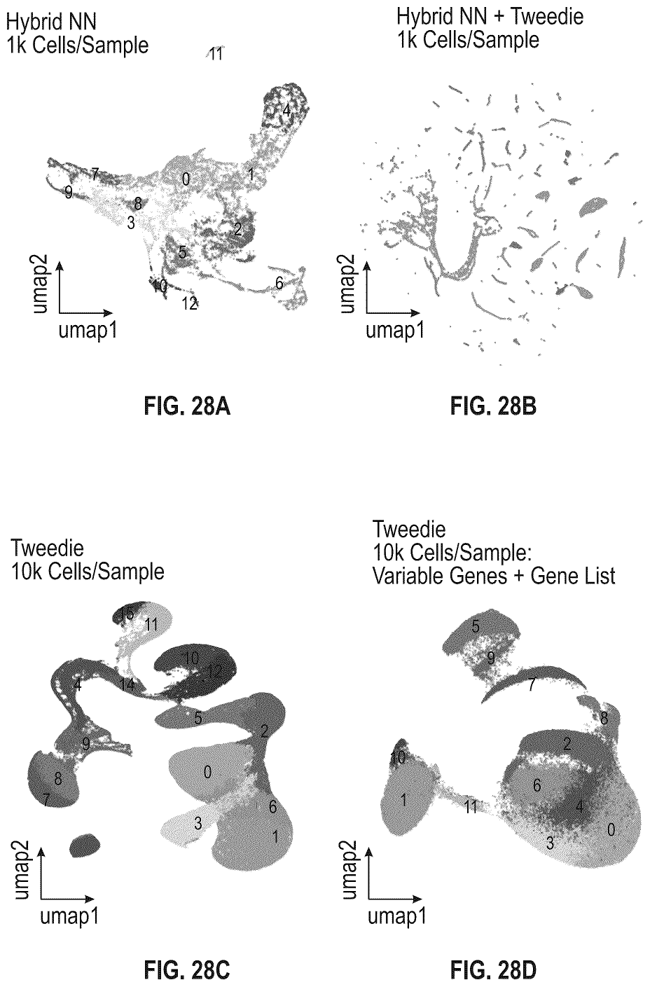

Several hybrid or singular neural network models were trained on T panel pseudofluorescence data intensities and the top 2000 most variable genes. For example, a negative log-likelihood loss based on the Tweedie distribution was used for training a continuous output model and cross entropy loss was used for training a binary output model. In comparison to other methods tried, the top 2000 variable genes were most accurately predicted using a non-hybrid neural network based entirely upon negative log-likelihood loss. The variation across models highlighted the suitability of different loss functions for different gene distributions. FIG. 28A-28D shows clustering of the top 2000 variable genes using the predicted expression from using several models (FIG. 28A hybrid neural network, FIG. 28B hybrid neural network and tweedie regression, FIG. 28C tweedie regression, FIG. 28D tweedie regression trained with variable genes and highly expressed genes). Performance of these models was indicated by inspecting differences between cell types on the UMAP representations, as it was expected that the known cell lineages (e.g. monocytes compared to T cells, B cells or NK cells) would separate in UMAP space. The Tweedie negative log-likelihood singular neural network performed best on 2000 variable genes (FIG. 28C).

### Example 9: Training a Machine Learning Model with Non-Negligible Genes

This example demonstrates the training of neural networks with all genes with more than 500 non-zero cells in the 53 individuals from the training data in Example 6. This set contained the genes from the highly expressed and high variable genes along with the remaining low variance genes. The low variance genes were not differentially expressed in the dataset, but they were included so that transfer learning could be used to apply the model to additional datasets where the genes may be differentially expressed.

For the full set of all genes with non-negligible expression (˜17,000 genes) the dataset was divided between highly expressed genes and low expressed genes based on their counts. A hybrid mean standard error (MSE)/tweedie neural network was trained to output four outputs: mean squared error loss for the highly expressed continuous output, negative log-likelihood loss (tweedie) for the low expression continuous output and two binary outputs for both sets which used cross entropy loss.

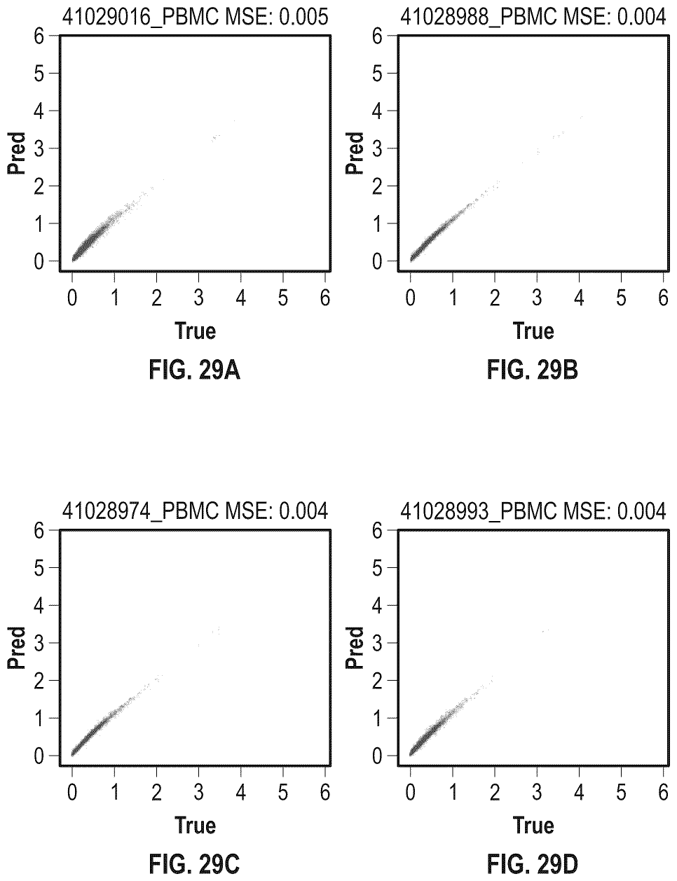

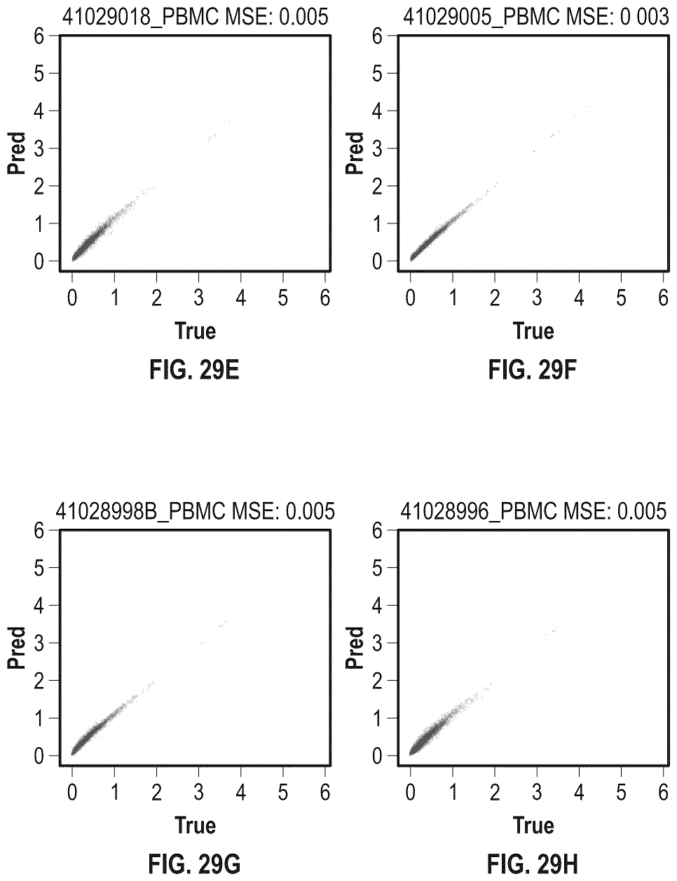

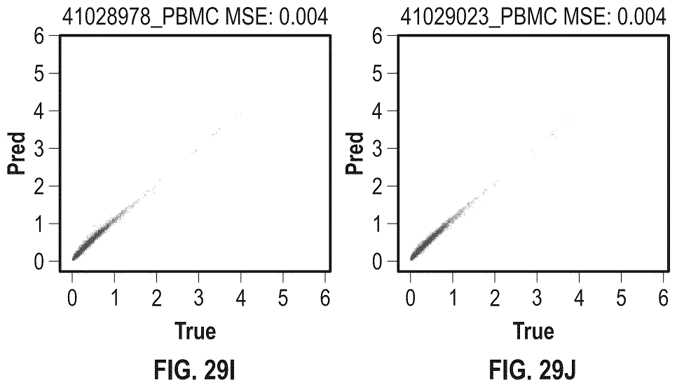

FIG. 29 shows evaluations of the prediction of single cell gene expression for ˜17,000 genes (A panel cells) compared to ground truth measured single cell gene expression. Each panel is a sample and MSE annotations represent mean squared error evaluation of each gene mean per cell type.

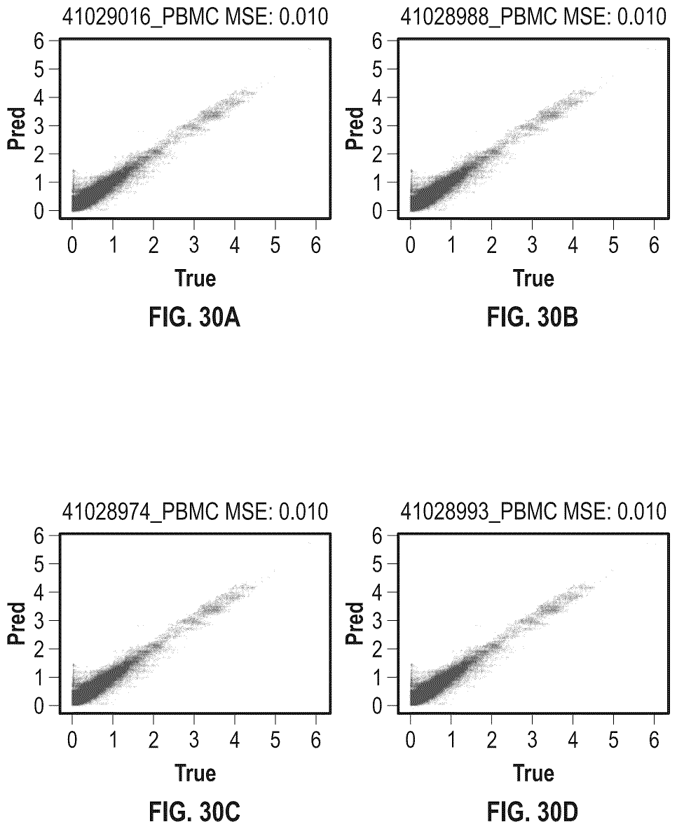

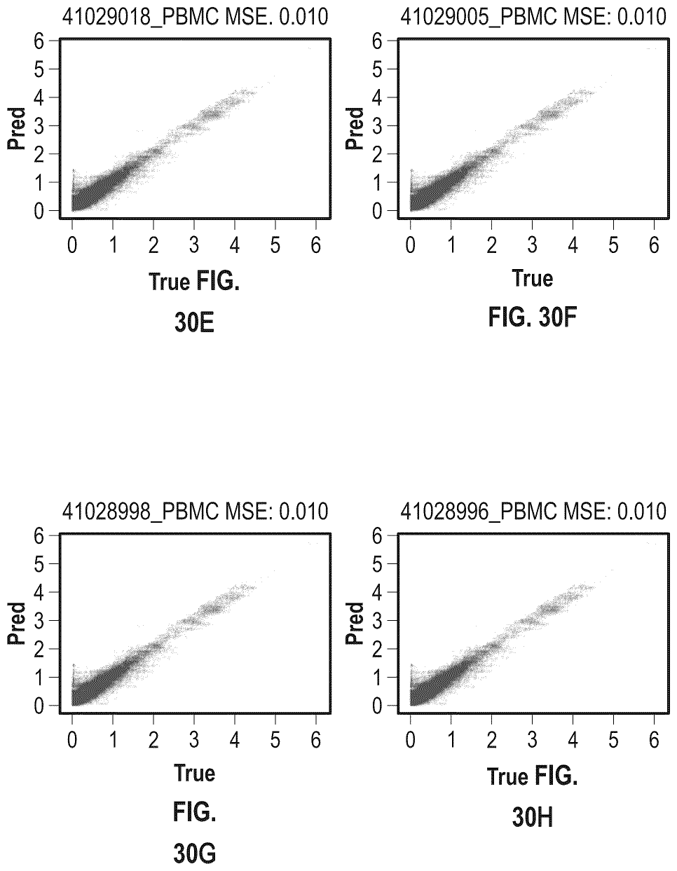

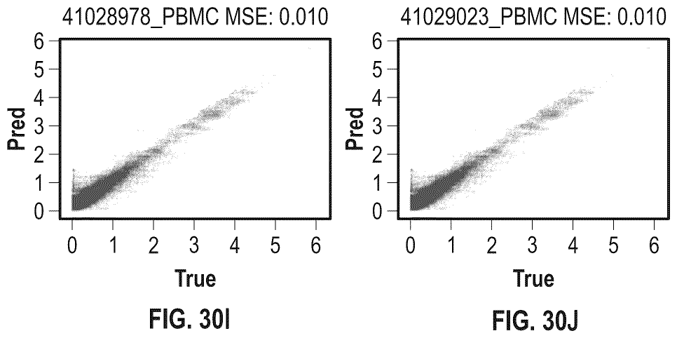

FIG. 30 shows evaluations of the prediction of single cell gene expression for ˜17,000 genes (T panel cells) compared to ground truth measured single cell gene expression. Each panel is a sample and MSE annotations represent mean squared error evaluation of each gene mean per cell type.

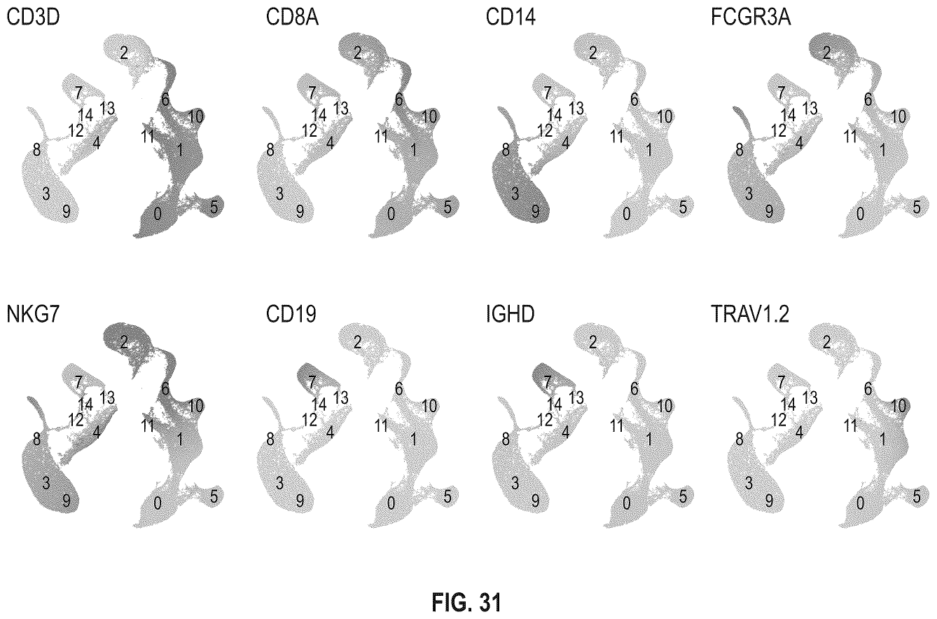

FIG. 31 shows the clustering of the predicted gene expression for the ˜17,000 genes in UMAP space according to expression of known population marker genes.

### Example 10: Immune Repertoire Prediction

This example demonstrates the training of machine learning models for immune repertoire prediction. This example shows the ability of the methods to predict gene expression and other single cell transcriptomic information, such as immune receptor diversity and/or prediction of clonal expansions.

The TCR sequences generated in Example 6 were filtered using scRepertoire and receptor clones were quantified at the amino acid level. The TCR data was filtered by removing: 1) any cell barcode with more than 2 immune receptor chains, and 2) any cell barcode with an NA value in at least one of the chains. After checking that the TCR barcodes exclusively associate with T cell populations identified in the clusters above, clone quantification across all cells and samples were generated. The clones were summarized at the level of amino acid sequence. This resulted in training data containing cell barcode, sample id, amino acid sequence, clonal quantification and the percentage of unique clones for a given donor. The same methods could be used to generate clones for the BCR sequences.

Every T cell in the training dataset from Example 6. was annotated with a clonal expansion status, reflecting whether or not its TCR clonotype was unique or common within its sample (whether or not its clonotype is public or private between individuals is irrelevant here). An XGBoost classifier was trained on individual pseudo-fluorescent cell classification memberships as described in Example 6 and the cell-matched clonal expansion annotations.

The expansion status was used as the primary target variable with the FSFC cell classification memberships as input variables to the model. The expansion status that was a simple boolean value indicating whether the clone abundance as determined from this TCR sequences was either equal to or greater than one. To select the features that were most significantly correlated to the expansion status, the Phi coefficient was computed for each feature. Populations with a |Phi|>0.01 were selected, yielding 84 populations with both positive and negative correlations to the expansion status.

Before training the model, the data was split into train and test sets in such a way that the test set contained cells from entirely different samples with respect to the train set. The training set contained 37 total samples, while the test set contained 16 total samples.

The hyperparameters of the XGboost classifier were tuned using Optuna-a Python library offering efficient and fast optimization. Optuna uses a Tree-based Parzen estimator under the hood-a Bayesian algorithm that is informed by previously sampled regions of the hyperparameter space. After a single run of Optuna on XGboost, optimal hyperparameters were found of 434 estimators, 0.02 learning rate, a max depth of 6, a min_child_weight of 4, a gamma of 0.367, a subsample parameter of 0.85, a colsample_bytree parameter of 0.642, a scale_pos_weight parameter of 4.56, and a max_delta_step of 1. The XGBoost classifier model was trained on the training set with these parameters.

The XGBoost classifier used a scale_pos_weight parameter to address class imbalance, which can lead to biased predicted probabilities. To achieve optimal performance, the predicted probability threshold was recalibrated. The default threshold was 0.5; however, by iteratively testing thresholds between 0 and 1 and computing the F1-score at each step, an optimal decision threshold of 0.7 was determined.

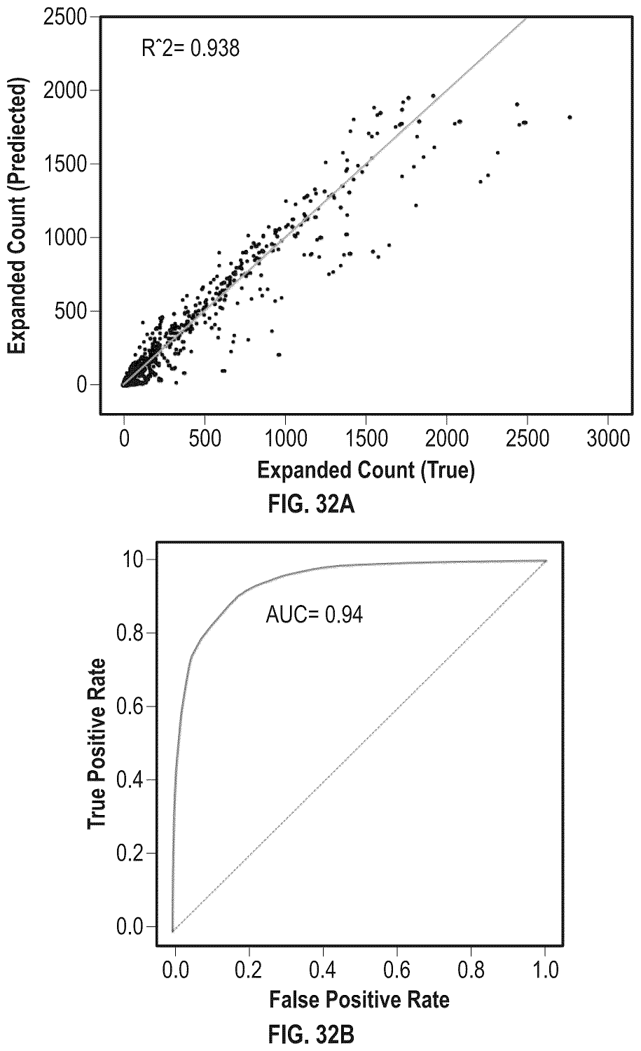

The clonality classifier model was evaluated on a hold-out test set of independent samples. To assess cell-wise prediction accuracy, ROC-AUC analysis was performed on predictions from pseudofluorescence data (pseudo-fluorescence cell classifications), indicating a strong model generalizability across samples at the cell-level. FIG. 32A shows the correlation between the predicted number of single cell clonal expansions compared to the true number of cells per clonotype per cell type. FIG. 32B shows a ROC-AUC analysis of the binary clonal expansion predictions. The evaluation resulted in an ROC-AUC score of 0.94, indicating a strong model generalizability across samples at the cell-level.

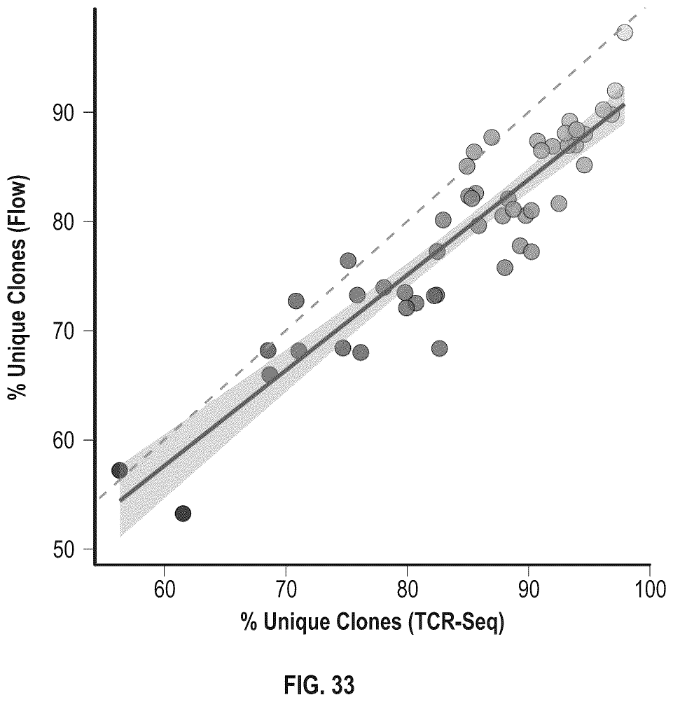

Predictions on the full dataset were aggregated into sample-level metrics in order to evaluate clonality predictions from matched FSFC fluorescence input data. These were the percent of the sample that was predicted to be expanded, and the percent of the sample that was actually expanded. FIG. 33 shows the predicted clonality compared to the ground truth clonality as measured using the TCR data.

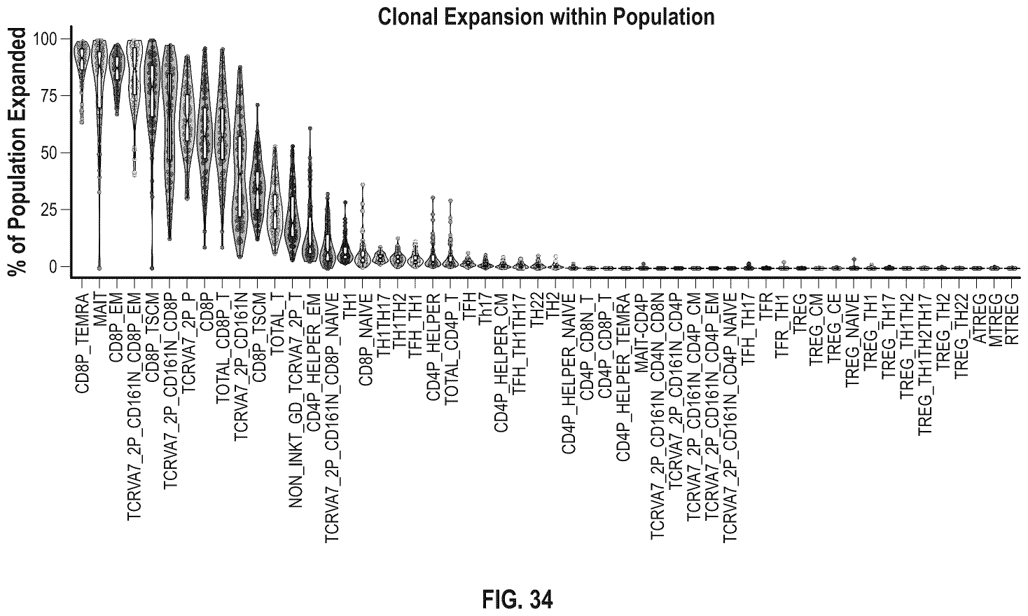

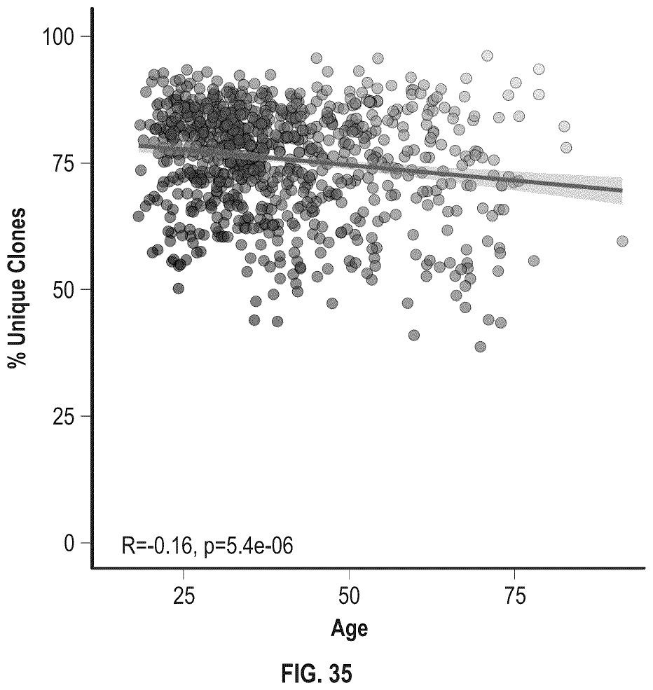

The clonality classifier model was deployed to predict single cell clonal expansion annotations for n=1,048 FSFC samples. These predictions were biological validated by manual assessment of clonality predictions based upon FSFC-defined cell classification memberships, where cell types expected to show the greatest clonal expansion indeed recorded the greatest levels of predicted clonal expansion (e.g. CD8+ TEMRA cells, CD8 EM cells) and vice versa (e.g. CD8 naive, CD4 helper naive, Treg) (FIG. 34). However, rare T cell subtypes that should represent clonally expanded cells (e.g. Th1, Tfh) were, in this example, predicted to have low to no clonal expansion, identifying potential areas for improvement in model performance. When predictions were aggregated to the sample level, per-donor clonality predictions were validated by the well-established (but slight) negative correlation between clonal diversity and biological age (FIG. 35).

1. A method for generating a single cell transcriptomic profile for a subject, comprising;
contacting at least a first aliquot of a sample from a subject with at least a first immunophenotyping panel to fluorescently label cells contained within the sample;
processing the fluorescently-labeled cell using a flow cytometer to generate fluorescent intensity data, or data derived therefrom, for a plurality of fluorescently-labeled cells from the sample;
providing only at least a subset of the fluorescent intensity data, or data derived therefrom for the plurality of fluorescently-labeled cells as input to a machine learning model trained using single cell transcriptomics data for a first plurality of cells and pseudo-fluorescent data for the first plurality of cells;
generating a predicted single cell transcriptomic value for the fluorescently-labeled cells using the trained machine learning model, thereby generating a single cell transcriptomic profile for the subject.

2. The method of claim 1, wherein the pseudo-fluorescent data for the first plurality of cells is generated by matching at least a subset of protein marker data for each cell in the first plurality of cells to fluorescent intensity data for each cell in a second plurality of cells.

3. The method of claim 1, wherein the pseudo-fluorescent data comprises pseudo-fluorescent marker data for the first plurality of cells and/or pseudo-fluorescent cell classifications for the first plurality of cells.

4. The method of claim 2, wherein the matching at least a subset of protein markers comprises transforming the protein marker data into pseudo-fluorescent marker data.

5. The method of claim 3, wherein the pseudo-fluorescent cell classification data are generated by assigning a pseudo-fluorescent cell classification to each cell related to the protein marker data that corresponds to each cell in the first plurality of cells.

6. The method of claim 1, wherein the single cell transcriptomics data for the first plurality of cells is generated using a method for characterizing each cell in the first plurality of cells by simultaneous detection of a plurality of protein marker data and single cell transcriptomic values.

7. The method of claim 6, wherein the single cell transcriptomics data comprises a single cell transcriptomic quantification and/or a clonal expansion status.

8. The method of claim 7, wherein the clonal expansion status for the first plurality of cells are generated from immune receptor profiling data.

9. The method of claim 8, wherein the immune receptor profiling data comprises TCR and/or BCR sequence data.

10. The method of claim 1, where in the single cell transcriptomic profile comprises single cell transcriptomic quantifications for a plurality of genes and/or clonal expansion statuses for a plurality of the fluorescently labeled cells.

11. The method of claim 1, wherein the flow cytometer is a full spectrum flow cytometer.

12. A method for training a machine learning model to generate a predicted single cell transcriptomic value, comprising;
collecting a population of cells comprising a first plurality of cells and a second plurality of cells;
obtaining single cell transcriptomic data and protein marker data for each cell in the first plurality of cells, wherein the single cell transcriptomic data comprise a single cell transcriptomic value for a plurality of genes;
obtaining fluorescent intensity data for each cell in the second plurality of cells generated using a flow cytometer to process fluorescently-labeled cells from the second plurality of cells;
generating pseudo-fluorescent data by matching at least a subset of the protein marker data for each cell in the first plurality of cells to the fluorescent intensity data for each cell in the second plurality of cells; and
training a machine learning model to generate a predicted single cell transcriptomic value, wherein the training is based on the transcriptomic data for at least a subset of the first plurality of cells and the pseudo-fluorescent data for the at least a subset of the first plurality of cells.

13. The method of claim 12, wherein obtaining single cell transcriptomic data and protein marker data for each cell in the first plurality of cells comprises simultaneous detection of a plurality of protein marker data and single cell transcriptomic values.

14. The method of claim 12, wherein the pseudo-fluorescent data comprises pseudo-fluorescent marker data for the first plurality of cells and/or pseudo-fluorescent cell classifications for the first plurality of cells.

15. The method of claim 12, wherein the matching at least a subset of protein markers comprises transforming the protein marker data into pseudo-fluorescent marker data.

16. The method of claim 14, wherein the pseudo-fluorescent cell classification data are generated by assigning a pseudo-fluorescent cell classification to each cell related to the protein marker data that corresponds to each cell in the first plurality of cells.

17. The method of claim 12, wherein the single cell transcriptomic data further comprises clonal expansion statuses for the first plurality of cells.

18. The method of claim 12, wherein the machine learning model has a flexible architecture.

19. The method of claim 12, wherein the single cell transcriptomic values comprise single cell transcriptomic quantifications for the plurality of genes.

20. The method of claim 19, wherein the method comprises selecting a machine learning model architecture from a group of machine learning architectures based on a distribution of the single cell transcriptomic quantifications for the plurality of genes.

21. The method of claim 20, wherein the group of machine learning architecture as comprises a hybrid classifier regression multilayer neural network, a regression multilayer neural network, a tweedie regression, a hybrid mean standard error (MSE)/Tweedie neural network and a gradient descent model.

22. The method of claim 19, wherein the machine learning model is trained to predict a single cell transcriptomic quantification from fluorescent intensity data.

23. The method of claim 20, wherein training the machine learning model comprises, minimizing one or more loss functions based on the machine learning model architecture.

24. The method of claim 23, wherein the one or more loss functions are selected from a group consisting of a log likelihood loss based on a tweedie distribution, negative log likelihood loss, mean squared error loss, mean absolute error (MAE), and cross entropy loss.

25. The method of claim 19, wherein the predicted single cell transcriptomic value comprises predicted single cell transcriptomic quantifications for the plurality of genes in the second plurality of cells.

26. The method of claim 12, wherein the single cell transcriptomic values comprise clonal expansion statuses.

27. The method of claim 26, wherein the machine learning model is a classifier model trained to predict clonal expansion statuses from fluorescence intensity data.

28. The method of claim 27, wherein the classifier model is an XGboost classifier.

29. The method of claim 26, wherein the predicted single cell transcriptomic value comprises predicted clonal expansion statuses for the second plurality of cells.
